(19) United States
(12) Patent Application Publication
(10) Pub. No.: US 2021/0378652 A1
(43) Pub. Date: Dec. 9, 2021

(54) CAMK2D ANTISENSE OLIGONUCLEOTIDES AND USES THEREOF

(71) Applicants: Bristol-Myers Squibb Company, Princeton, NJ (US); Roche Innovation Center Copenhagen A/S, Hørsholm (DK)

(72) Inventors: Richard E. OLSON, Cambridge, MA (US); Brian R. ANDERSON, Princeton, NJ (US); Peter HAGEDORN, Hørsholm (DK); Marianne Lerbech JENSEN, Køge (DK); Ivar M. MCDONALD, East Haddam, CT (US); Stephen E. MERCER, Middletown, CT (US)

(21) Appl. No.: 17/303,890

(22) Filed: Jun. 9, 2021

Related U.S. Application Data
(62) Division of application No. 16/281,138, filed on Feb. 21, 2019, now Pat. No. 11,083,444.
(60) Provisional application No. 62/633,502, filed on Feb. 21, 2018.
(60) Provisional application No. 62/635,954, filed on Feb. 27, 2018.
(60) Provisional application No. 62/665,998, filed on May 2, 2018.
(60) Provisional application No. 62/778,679, filed on Dec. 12, 2018.

(51) Int. Cl. A61B 17/02; A61F 2/44; A61B 1/32

(52) U.S. Cl. CPC A61B 17/0206; A61B 90/50; A61B 1/32; A61F 2/4455

(57) ABSTRACT

The present disclosure relates to antisense oligonucleotides, which target CAMK2D mRNA in a cell, leading to reduced expression of CAMK2D protein. Reduction of CAMK2D protein expression is beneficial for the treatment of certain medical disorders, e.g., cardiovascular-related diseases or disorders.

### CROSS-REFERENCE TO RELATED APPLICATIONS

This application is a divisional of U.S. application Ser. No. 16/282,138, filed Feb. 21, 2019, which claims priority benefit of U.S. Provisional Application Nos. 62/633,502, filed Feb. 21, 2018; 62/635,954, filed Feb. 27, 2018; 62/665,998 filed May 2, 2018; and 62/778,679, filed Dec. 12, 2018, each of which is incorporated herein by reference in its entirety.

### REFERENCE TO SEQUENCE LISTING SUBMITTED ELECTRONICALLY VIA EFS-WEB

The content of the electronically submitted sequence listing (Name: 3338-1020006_SL_ST25.txt, Size: 746,343 bytes; and Date of Creation: Jun. 9, 2021) submitted in this application is incorporated herein by reference in its entirety.

### FIELD OF DISCLOSURE

The present disclosure relates to antisense oligomeric compounds (ASOs) that target calcium/calmodulin-dependent protein kinase type II delta (CAMK2D) transcript in a cell, leading to reduced expression of CAMK2D protein. Reduction of CAMK2D protein expression can be beneficial for a range of medical disorders, such as cardiovascular-related diseases or disorders.

### BACKGROUND

Calcium/calmodulin (Ca²⁺/CaM)-dependent serine/threonine kinases (CaMKs) constitute a family of 81 proteins in the human proteasome that play a central role in cellular signaling by transmitting Ca²⁺ signals. Four CaMKII isozymes (α, β, γ, and δ), in addition to about 30 splice variants, are expressed in humans. Braun, A. P., et al., Annual Review of Physiology 57:417-445 (1995). Of these, CaMKIIδ (“CAMK2D”) protein is the most abundant isoform in the heart and plays an important role in the excitation-contraction coupling (ECC) and relaxation processes of normal cardiac physiology. Mattiazzi A., et al., Am J Physiol Heart Circ Physiol 308:H1177-H1191 (2015). CAMK2D activity has also been described as being important in the recovery process after certain heart related injury (e.g., ischemia-reperfusion injury). Said M., el al., Am J Physio Heart Circ Physiol 285:H1198-205 (2003).

Despite various scientific advancements, heart-related diseases remain the leading cause of death for both men and women worldwide. The American Heart Association estimates that by 2030, nearly 40% of the U.S. population would have some form of a cardiovascular disease and the direct medical costs are projected to reach $818 billion. See Benjamin, E. J., et al., Circulation 135:e146-e603 (2017). However, Mattiazzi et al. notes that “[t]he ubiquitous nature of CaMKII and its effects on different protein targets challenge the use of CaMKII inhibitors as a therapeutic tool.” Am J Physiol Heart Circ Physiol 308:H1177-H1191 (2015). Therefore, new treatment options that are much more robust and cost-effective are highly desirable.

### SUMMARY OF DISCLOSURE

The present disclosure is directed to an antisense oligonucleotide (ASO) comprising, consisting essentially of, or consisting of the contiguous nucleotide sequence of 10 to 30 nucleotides in length that is complementary, such as fully complementary, to a nucleic acid sequence within a calcium/calmodulin-dependent protein kinase type II delta (CAMK2D) transcript. In some embodiments, the ASO of the present disclosure, or contiguous nucleotide sequence thereof, is at least about 80%, at least about 85%, at least about 90%, at least about 95%, or about 100% complementary to the nucleic acid sequence within the CAMK2D transcript. In some embodiments, the CAMK2D transcript is selected from the group consisting of SEQ ID NO: 1 and SEQ ID NO. 2.

In some embodiments, the ASO described herein is capable of reducing CAMK2D protein expression in a human cell (e.g., HEK293 cell) which is expressing the CAMK2D protein. In some embodiments, the CAMK2D protein expression is reduced by at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to CAMK2D protein expression in a human cell that is not exposed to the ASO.

In some embodiments, the ASO is capable of reducing CAMK2D transcript (e.g., mRNA) expression in a human cell (e.g., HEK293 cell), which is expressing the CAMK2D transcript. In some embodiments, the CAMK2D transcript expression is reduced by at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to CAMK2D transcript expression in a human cell that is not exposed to the ASO.

In some embodiments, the ASO disclosed herein is a gapmer. In some embodiments, the ASO has a design of LLLDₙLLL, LLLLDₙLLLL, or LLLLLDₙLLLLL, wherein the L is a nucleoside analog, the D is DNA, and n can be any integer between 4 and 24. In some embodiments, n can be any integer between 6 and 14. In some embodiments, n can be any integer between 8 and 12.

In some embodiments, the nucleoside analog of the ASO disclosed herein comprises a 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluoro-RNA; 2′-fluoro-DNA, arabino nucleic acid (ANA); 2′-fluoro-ANA; or bicyclic nucleoside analog (LNA). In some embodiments, one or more of the nucleoside analog of the ASO is a sugar modified nucleoside. In some embodiments, the sugar modified nucleoside is an affinity enhancing 2′ sugar modified nucleoside. In some embodiments, one or more of the nucleoside analog comprises a nucleoside comprising a bicyclic sugar. In some embodiments, the affinity enhancing 2′ sugar modified nucleoside is an LNA. In some embodiments, the LNA is selected from the group consisting of constrained ethyl nucleoside (cEt), 2′,4′-constrained 2′-O-methoxyethyl (cMOE), α-L-LNA, β-D-LNA, 2′-O,4′-C-ethylene-bridged nucleic acids (ENA), amino-LNA, oxy-LNA, thio-LNA, or any combination thereof. In some embodiments, the ASO comprises one or more 5′-methyl-cytosine nucleobases.

In some embodiments, the ASO described herein is capable of (i) reducing an mRNA level encoding CAMK2D inhuman Inducible Pluripotent Stem Cell-Derived Cardiomyocytes (hiPSC-CM); (ii) reducing a protein level of CAMK2D in hiPSC-CM; (iii) reducing, ameliorating, or treating one or more symptoms of a cardiovascular disease or disorder, and (iv) any combination thereof.

In some embodiments, the contiguous nucleotide sequence of the ASO is complementary to a nucleic acid sequence comprising (i) nucleotides 625-842 of SEQ ID NO: 1; (ii) nucleotides 1,398-59,755 of SEQ ID NO: 1; (iii) nucleotides 61,817-104,725 of SEQ ID NO: 1; (iv) nucleotides 112,162-118,021 of SEQ ID NO: 1; (v) nucleotides 119,440-135,219 of SEQ ID NO: 1; (vi) nucleotides 137,587-157,856 of SEQ ID NO: 1; (vii) nucleotides 159,191-266,174 of SEQ ID NO: 1; or (viii) nucleotides 272,788-310,949 of SEQ ID NO: 1. In some embodiments, the contiguous nucleotide sequence of the ASO is complementary to a nucleic acid sequence comprising (i) nucleotides 675-792 of SEQ ID NO: 1; (ii) nucleotides 1,448-59,705 of SEQ ID NO: 1; (iii) nucleotides 61,867-104,675 of SEQ ID NO: 1; (iv) nucleotides 112,212-117,971 of SEQ ID NO: 1; (v) nucleotides 119,490-135,169 of SEQ ID NO: 1; (vi) nucleotides 137,637-157,806 of SEQ ID NO: 1; (vii) nucleotides 159,241-266,124 of SEQ ID NO: 1; or (viii) nucleotides 272,838-310,899 of SEQ ID NO: 1. In some embodiments, the contiguous nucleotide sequence of the ASO is complementary to a nucleic acid sequence comprising (i) nucleotides 725-742 of SEQ ID NO: 1; (ii) nucleotides 1,498-59,655 of SEQ ID NO: 1; (iii) nucleotides 61,917-104,625 of SEQ ID NO: 1; (iv) nucleotides 112,262-117,921 of SEQ ID NO: 1; (v) nucleotides 119,540-135,119 of SEQ ID NO: 1; (vi) nucleotides 137,687-157,756 of SEQ ID NO: 1; (vii) 159,291-266,074 of SEQ ID NO: 1; or (viii) nucleotides 272,888-310,849 of SEQ ID NO: 1.

In some embodiments, the contiguous nucleotide sequence of the ASO comprises SEQ ID NO: 4 to SEQ ID NO: 1713 with one or two mismatches. In some embodiments, the contiguous nucleotide sequence of the ASO comprises the nucleotide sequence selected from the sequences in FIGS. 1A and 1B (SEQ ID NO: 4 to SEQ ID NO: 1713). In some embodiments, the contiguous nucleotide sequence of the ASO comprises SEQ ID NO: 25, SEQ ID NO: 27, SEQ ID NO: 114, SEQ ID NO: 158, SEQ ID NO: 190, SEQ ID NO: 327, SEQ ID NO: 463, SEQ ID NO: 513, SEQ ID NO: 516, SEQ ID NO: 519, SEQ ID NO: 657, SEQ ID NO: 659, SEQ ID NO: 827, SEQ ID NO: 1249, SEQ ID NO: 1326, SEQ ID NO: 1409, SEQ ID NO: 1524, SEQ ID NO: 1530, SEQ ID NO: 1662, or SEQ ID NO: 1676. In some embodiments, the contiguous nucleotide sequence of the ASO comprises SEQ ID NO: 55, SEQ ID NO: 61, SEQ ID NO: 63, SEQ ID NO: 71, SEQ ID NO: 75, SEQ ID NO: 79, SEQ ID NO: 84, SEQ ID NO: 85, SEQ ID NO: 92, SEQ ID NO: 102, SEQ ID NO: 105, SEQ ID NO: 128, SEQ ID NO: 130, SEQ ID NO. 133, SEQ ID NO: 138, SEQ ID NO: 161, SEQ ID NO: 178, SEQ ID NO: 180, SEQ ID NO: 186, SEQ ID NO: 195, SEQ ID NO: 200, SEQ ID NO: 202, SEQ ID NO: 234, SEQ ID NO: 264, SEQ ID NO: 387, SEQ ID NO: 390, SEQ ID NO: 396, SEQ ID NO: 441, SEQ ID NO: 446, SEQ ID NO: 457, SEQ ID NO: 467, SEQ ID NO: 523, SEQ ID NO: 524, SEQ ID NO: 636, SEQ ID NO: 640, SEQ ID NO: 700, SEQ ID NO: 740, SEQ ID NO: 832, SEQ ID NO: 965, SEQ ID NO: 1015, SEQ ID NO: 1065, SEQ ID NO: 1071, SEQ ID NO: 1155, SEQ ID NO: 1475, SEQ ID NO: 1508, SEQ ID NO: 1685, SEQ ID NO: 1686, SEQ ID NO: 1687, SEQ ID NO: 1688, or SEQ ID NO: 1690.

In some embodiments, the ASO of the present disclosure has a design selected from the group consisting of the designs in FIG. 3, wherein the upper letter is a sugar modified nucleoside and the lower case letter is DNA.

In some embodiments, the ASO disclosed herein is capable of reducing expression of CAMK2D protein in a hiPSC-CM cell which is expressing the CAMK2D protein. In some embodiments, the expression of CAMK2D protein is reduced by at least about 20%, at least about 30%, at least about 40%, at least about 50%, at least about 60%, at least about 70%, at least about 80%, at least about 90%, or about 100% compared to a cell not exposed to the ASO. In some embodiments, the ASO is capable of reducing expression of CAMK2D transcript (e.g., mRNA) in a hiPSC-CM cell which is expressing the CAMK2D transcript. In some embodiments, the expression of CAMK2D transcript is reduced by at least about 20%, at least about 30%, at least about 40%, at least about 50%, at least about 60%, at least about 70%, at least about 80%, at least about 90%, or about 100% compared to a cell not exposed to the ASO.

In some embodiments, the ASO has from 14 to 20 nucleotides in length. In some embodiments, the nucleotide sequence of the ASO comprises one or more modified internucleoside linkage. In some embodiments, at least 75%, at least 80%, at least 85%, at least 90%, at least 95%, or 100% of internucleoside linkages are modified. In certain embodiments, each of the internucleotide linkages in the ASO of the present disclosure is a phosphorothioate linkage.

The present disclosure also provides a conjugate comprising the ASO as disclosed herein, wherein the ASO is covalently attached to at least one non-nucleotide or non-polynucleotide moiety. In some embodiments, the non-nucleotide or non-polynucleotide moiety comprises a protein, a fatty acid chain, a sugar residue, a glycoprotein, a polymer, or any combinations thereof.

Also provided herein is a pharmaceutical composition comprising the ASO or the conjugate as disclosed herein and a pharmaceutically acceptable diluent, carrier, salt, or adjuvant. In certain embodiments, a pharmaceutically acceptable salt comprises a sodium salt, a potassium salt, or an ammonium salt. In some embodiments, the pharmaceutical composition further comprises at least one further therapeutic agent. In some embodiments, the further therapeutic agent is a CAMK2D antagonist. In some embodiments, the CAMK2D antagonist is an anti-CAMK2d antibody or fragment thereof.

The present disclosure further provides a kit comprising the ASO, the conjugate, or the pharmaceutical composition as disclosed herein, and instructions for use. Also disclosed is a diagnostic kit comprising the ASO, the conjugate, or the pharmaceutical composition of the present disclosure, and instructions for use.

The present disclosure is also directed method of inhibiting or reducing CAMK2D protein expression in a cell, comprising administering the ASO, the conjugate, or the pharmaceutical composition disclosed herein to the cell expressing CAMK2D protein, wherein the CAMK2D protein expression in the cell is inhibited or reduced after the administration. In some aspect, the present disclosure is directed to an in vitro method of inhibiting or reducing CAMK2D protein expression in a cell, comprising contacting the ASO, the conjugate, or the pharmaceutical composition disclosed herein to the cell expressing CAMK2D protein, wherein the CAMK2D protein expression in the cell is inhibited or reduced after the contacting. In some embodiments, the ASO inhibits or reduces expression of CAMK2D transcript (e.g., mRNA) in the cell after the administration. In some embodiments, the expression of CAMK2D transcript (e.g., mRNA) is reduced by at least about 20%, at least about 30%, at least about 40%, at least about 50%, at least about 60%, at least about 70%, at least about 80%, at least about 90%, or about 100% after the administration compared to a cell not exposed to the ASO. In some embodiments, the expression of CAMK2D protein is reduced by at least about 60%, at least about 70%, at least about 75%, at least about 80%, at least about 85%, at least about 90%, at least about 95%, at least about 96%, at least about 97%, at least about 98%, at least about 99%, or about 100% after the administration compared to a cell not exposed to the ASO. In some embodiments, the cell is a cardiac cell, e.g., hiPSC-CM.

Provided herein is a method of reducing, ameliorating, or treating one or more symptoms of a cardiovascular disease or disorder in a subject in need thereof, comprising administering an effective amount of the ASO, the conjugate, or the pharmaceutical composition of the present disclosure to the subject. The present disclosure also provides the use of the ASO, the conjugate, or the pharmaceutical composition disclosed herein for the manufacture of a medicament. In some embodiments, the medicament is for the treatment of a cardiovascular disease or disorder in a subject in need thereof. In some embodiments, the ASO, the conjugate, or the pharmaceutical composition of the present disclosure is for use in therapy. In some embodiments, the ASO, the conjugate, or the pharmaceutical composition disclosed herein is for use in therapy of a cardiovascular disease or disorder in a subject in need thereof.

In some embodiments, the cardiovascular disease or disorder comprises a coronary artery disease, stroke, heart failure, hypertensive heart disease, rheumatic heart disease, cardiomyopathy, heart arrhythmia, congenital heart disease, valvular heart disease carditis, aortic aneurysms, peripheral artery disease, thromboembolic disease, venous thrombosis, or any combination thereof. In some embodiments, the cardiovascular disease or disorder is a heart failure. In some embodiments, the heart failure comprises a left-sided heart failure, a right-sided heart failure, a congestive heart failure, a heart failure with reduced ejection fraction (HFrEF), a heart failure with preserved ejection fraction (HFpEF), a heart failure with mid-range ejection fraction (HFmrEF), a hypertrophic cardiomyopathy (HCM), a hypertensive heart disease (HHD), or hypertensive hypertrophic cardiomyopathy.

In some embodiments, the subject is a human. In some embodiments, the ASO, the conjugate, or the pharmaceutical composition of the present disclosure is administered intracardially, orally, parenterally, intrathecally, intra-cerebroventricularly, pulmorarily, topically, or intraventricularly.

### BRIEF DESCRIPTION OF FIGURES

FIGS. 1A and 1B show exemplary ASOs targeting the CAMK2D pre-mRNA. FIG. 1A shows the ASOs targeting a single site within the CAMK2D pre-mRNA. FIG. 1B shows the ASOs targeting multiple sites (i.e., two or three) within the CAMK2D pre-mRNA. Each column of FIGS. 1A and 1B show the SEQ ID number designated for the sequence only of the ASO, the target start and end positions on the CAMK2D pre-mRNA sequence (for FIG. 1B, the multiple target sites are identified as #1, #2, or #3), the ASO sequence without any particular design or chemical structure, the ASO number (ASO No.), and the ASO sequence with a chemical structure.

FIG. 2 shows both the percent reduction of CAMK2D mRNA expression in HEK293 cells (y-axis) and the relative position of the ASOs on the CAMK2D) transcript (x-axis). Each circle represents an individual ASO. As further described in Example 2, the HEK293 cells were treated with 25 μM of ASO and the CAMK2D mRNA expression (normalized to GAPDH) is shown as a percent of the control.

FIG. 3 shows certain exemplary ASOs with their design. Each column of FIG. 3 shows the SEQ ID NO for the ASO sequence only, the target start and end positions on the CAMK2D pre-mRNA sequence (where the ASO binds to multiple sites (see FIG. 1B), exemplary target start and end positions are provided), the ASO design number (DES No.), the ASO sequence with a design, and the ASO number (ASO No.).

FIG. 4 shows the percent reduction of CAMK2D mRNA expression in both HEK293 cells and human inducible pluripotent stem cell-derived cardiomyocytes (hiPSC-CM) after in vitro culture with various ASOs as described in Examples 2 and 3. The cells were treated with 25 sM (HEK293) or 500 nM (hiPSC-CM) of ASO and the CAMK2D mRNA expression (normalized to GAPDH) is shown as a percent of the control. Where no value is provided, the particular ASO was not tested under the particular conditions.

FIG. 5 shows the potency of exemplary ASOs on CAMK2D mRNA expression level in C57BL/6JBom mice one week after subcutaneous administration. CAMK2D mRNA expression level was normalized to GAPDH and then shown relative to the control group (i.e., saline treated samples).

### DETAILED DESCRIPTION OF DISCLOSURE

### I. Definitions

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects. A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their System International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). For example, if it is stated that “the ASO reduces expression of CAMK2d protein in a cell following administration of the ASO by at least about 60%,” it is implied that the CAMK2D levels are reduced by a range of 50% to 70%.

The term “nucleic acids” or “nucleotides” is intended to encompass plural nucleic acids. In some embodiments, the term “nucleic acids” or “nucleotides” refers to a target sequence, e.g., pre-mRNAs, mRNAs, or DNAs in vivo or in vitro. When the term refers to the nucleic acids or nucleotides in a target sequence, the nucleic acids or nucleotides can be naturally occurring sequences within a cell. In other embodiments, “nucleic acids” or “nucleotides” refer to a sequence in the ASOs of the disclosure. When the term refers to a sequence in the ASOs, the nucleic acids or nucleotides are not naturally occurring, i.e., chemically synthesized, enzymatically produced, recombinantly produced, or any combination thereof. In one embodiment, the nucleic acids or nucleotides in the ASOs are produced synthetically or recombinantly, but are not a naturally occurring sequence or a fragment thereof. In another embodiment, the nucleic acids or nucleotides in the ASOs are not naturally occurring because they contain at least one nucleotide analog that is not naturally occurring in nature. The term “nucleic acid” or “nucleoside” refers to a single nucleic acid segment, e.g., a DNA, an RNA, or an analog thereof, present in a polynucleotide. “Nucleic acid” or “nucleoside” includes naturally occurring nucleic acids or non-naturally occurring nucleic acids. In some embodiments, the terms “nucleotide”, “unit” and “monomer” are used interchangeably. It will be recognized that when referring to a sequence of nucleotides or monomers, what is referred to is the sequence of bases, such as A, T, G, C or U, and analogs thereof.

The term “nucleotide” as used herein, refers to a glycoside comprising a sugar moiety, a base moiety and a covalently linked group (linkage group), such as a phosphate or phosphorothioate internucleotide linkage group, and covers both naturally occurring nucleotides, such as DNA or RNA, and non-naturally occurring nucleotides comprising modified sugar and/or base moieties, which are also referred to as “nucleotide analogs” herein. Herein, a single nucleotide (unit) can also be referred to as a monomer or nucleic acid unit. In certain embodiments, the term “nucleotide analogs” refers to nucleotides having modified sugar moieties. Non-limiting examples of the nucleotides having modified sugar moieties (e.g., LNA) are disclosed elsewhere herein. In other embodiments, the term “nucleotide analogs” refers to nucleotides having modified nucleobase moieties. The nucleotides having modified nucleobase moieties include, but are not limited to, 5-methyl-cytosine, isocytosine, pseudoisocytosine, 5-bromouracil, 5-propynyluracil, 6-aminopurine, 2-aminopurine, inosine, diaminopurine, and 2-chloro-6-aminopurine.

The term “nucleoside” as used herein is used to refer to a glycoside comprising a sugar moiety and a base moiety, and can therefore be used when referring to the nucleotide units, which are covalently linked by the internucleotide linkages between the nucleotides of the ASO. In the field of biotechnology, the term “nucleotide” is often used to refer to a nucleic acid monomer or unit. In the context of an ASO, the term “nucleotide” can refer to the base alone, i.e., a nucleobase sequence comprising cytosine (DNA and RNA), guanine (DNA and RNA), adenine (DNA and RNA), thymine (DNA) and uracil (RNA), in which the presence of the sugar backbone and internucleotide linkages are implicit. Likewise, particularly in the case of oligonucleotides where one or more of the internucleotide linkage groups are modified, the term “nucleotide” can refer to a “nucleoside.” For example the term “nucleotide” can be used, even when specifying the presence or nature of the linkages between the nucleosides.

The term “nucleotide length” as used herein means the total number of the nucleotides (monomers) in a given sequence. For example, the sequence of tacatattatattactcctc (SEQ ID NO: 158) has 20 nucleotides; thus the nucleotide length of the sequence is 20. The term “nucleotide length” is therefore used herein interchangeably with “nucleotide number.”

As one of ordinary skill in the art would recognize, the 5′ terminal nucleotide of an oligonucleotide does not comprise a 5′ internucleotide linkage group, although it can comprise a 5′ terminal group.

As used herein, the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C₁-C₈ alkyl groups are methyl, ethyl, propyl, isopropyl, butyl, isobutyl, tert-butyl, the isomeric pentyls, the isomeric hexyls, the isomeric heptyls and the isomeric octyls, particularly methyl, ethyl, propyl, butyl and pentyl. Particular examples of alkyl are methyl. Further examples of alkyl are mono, di or trifluoro methyl, ethyl or propyl, such as cyclopropyl (cPr), or mono, di or tri fluoro cycloproyl.

The term “alkoxy”, alone or in combination, signifies a group of the formula alkyl-O— in which the term “alkyl” has the previously given significance, such as methoxy, ethoxy, n-propoxy, isopropoxy, n-butoxy, isobutoxy, sec.butoxy and tert.butoxy. Particular “alkoxy” are methoxy.

The term “oxy”, alone or in combination, signifies the —O— group.

The term “alkenyl”, alone or in combination, signifies a straight-chain or branched hydrocarbon residue comprising an olefinic bond and up to 8, preferably up to 6, particularly preferred up to 4 carbon atoms. Examples of alkenyl groups are ethenyl, 1-propenyl, 2-propenyl, isopropenyl, 1-butenyl, 2-butenyl, 3-butenyl and isobutenyl.

The term “alkynyl”, alone or in combination, signifies a straight-chain or branched hydrocarbon residue comprising a triple bond and up to 8, preferably up to 6, particularly preferred up to 4 carbon atoms.

The terms ““halogen”” or ““halo””, alone or in combination, signifies fluorine, chlorine, bromine or iodine and particularly fluorine, chlorine or bromine, more particularly fluorine and chlorine, such as fluorine. The term “halo”, in combination with another group, denotes the substitution of said group with at least one halogen, particularly substituted with one to five halogens, particularly one to four halogens, i.e., one, two, three or four halogens. The terms “hydroxyl” and “hydroxy”, alone or in combination, signify the —OH group.

The terms “thiohydroxyl” and “thiohydroxy”, alone or in combination, signify the —SH group.

The term “carbonyl”, alone or in combination, signifies the —C(O)— group.

The term “carboxy” or “carboxyl”, alone or in combination, signifies the —COOH group.

The term “amino”, alone or in combination, signifies the primary amino group (—NH2), the secondary amino group (—NH—), or the tertiary amino group (—N—).

The term “alkylamino”, alone or in combination, signifies an amino group as defined above substituted with one or two alkyl groups as defined above.

The term “aminocarbonyl, alone or in combination, signifies the —C(O)—NH2 group.

The term “sulfonyl”, alone or in combination, means the —SO2 group.

The term “sulfinyl”, alone or in combination, signifies the —SO— group.

The term “sulfanyl”, alone or in combination, signifies the —S— group.

The term “cyano”, alone or in combination, signifies the —CN group.

The term “azido”, alone or in combination, signifies the —N3 group.

The term “nitro”, alone or in combination, signifies the NO2 group.

The term “formyl” alone or in combination, signifies the —C(O)H group.

The term “aryl”, alone or in combination, denotes a monovalent aromatic carbocyclic mono- or bicyclic ring system comprising 6 to 10 carbon ring atoms, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples of aryl include phenyl and naphthyl, in particular phenyl.

The term “heteroaryl”, alone or in combination, denotes a monovalent aromatic heterocyclic mono- or bicyclic ring system of 5 to 12 ring atoms, comprising 1, 2, 3 or 4 heteroatoms selected from N, O and S, the remaining ring atoms being carbon, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples of heteroaryl include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, azepinyl, diazepinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, quinoxalinyl, carbazolyl, or acridinyl.

The term “heterocycle”, alone or in combination, denotes a monovalent non-aromatic heterocyclic mono- or bicyclic ring system of 5 to 12 ring atoms, comprising 1, 2, 3 or 4 heteroatoms selected from N, O and S, the remaining ring atoms being carbon, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl.

The term “protecting group” alone or in combination, signifies a group which selectively blocks a reactive site in a multifunctional compound such that a chemical reaction can be carried out selectively at another unprotected reactive site. Protecting groups can be removed. Exemplary protecting groups are amino-protecting groups, carboxy-protecting groups or hydroxy-protecting groups.

If one of the starting materials or compounds of the invention contain one or more functional groups which are not stable or are reactive under the reaction conditions of one or more reaction steps, appropriate protecting groups (as described e.g., in “Protective Groups in Organic Chemistry” by T. W. Greene and P. G. M. Wuts, 3rd Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compounds described herein can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

The term “asymmetric carbon atom” means a carbon atom with four different substituents. According to the Cahn-Ingold-Prelog Convention an asymmetric carbon atom can be of the “R” or “S” configuration.

As used herein, the term “bicyclic sugar” refers to a modified sugar moiety comprising a 4 to 7 membered ring comprising a bridge connecting two atoms of the 4 to 7 membered ring to form a second ring, resulting in a bicyclic structure. In some embodiments, the bridge connects the C2′ and C4′ of the ribose sugar ring of a nucleoside (i.e., 2′4′ bridge), as observed in LNA nucleosides.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, untranslated regions (“UTRs”), and the like, are not part of a coding region. The boundaries of a coding region are typically determined by a start codon at the 5 terminus, encoding the amino terminus of the resultant polypeptide, and a translation stop codon at the 3′ terminus, encoding the carboxyl terminus of the resulting polypeptide.

The term “non-coding region” as used herein means a nucleotide sequence that is not a coding region. Examples of non-coding regions include, but are not limited to, promoters, ribosome binding sites, transcriptional terminators, introns, untranslated regions (“UTRs”), non-coding exons and the like. Some of the exons can be wholly or part of the 5′ untranslated region (5′ UTR) or the 3′ untranslated region (3′ UTR) of each transcript. The untranslated regions are important for efficient translation of the transcript and for controlling the rate of translation and half-life of the transcript.

The term “region” when used in the context of a nucleotide sequence refers to a section of that sequence. For example, the phrase “region within a nucleotide sequence” or “region within the complement of a nucleotide sequence” refers to a sequence shorter than the nucleotide sequence, but longer than at least 10 nucleotides located within the particular nucleotide sequence or the complement of the nucleotides sequence, respectively. The term “sub-sequence” or “subsequence” can also refer to a region of a nucleotide sequence.

The term “downstream,” when referring to a nucleotide sequence, means that a nucleic acid or a nucleotide sequence is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence.

As used herein, the term “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites, UTRs, and stem-loop structures. If a coding region is intended for expression in a eukaryotic cell, a polyadenylation signal and transcription termination sequence will usually be located 3′ to the coding sequence.

The term “transcript” as used herein can refer to a primary transcript that is synthesized by transcription of DNA and becomes a messenger RNA (mRNA) after processing, i.e., a precursor messenger RNA (pre-mRNA), and the processed mRNA itself. The term “transcript” can be interchangeably used with “pre-mRNA” and “mRNA.” After DNA strands are transcribed to primary transcripts, the newly synthesized primary transcripts are modified in several ways to be converted to their mature, functional forms to produce different proteins and RNAs such as mRNA, tRNA, rRNA, IncRNA, miRNA and others. Thus, the term “transcript” can include exons, introns, 5′ UTRs, and 3′ UTRs.

The term “expression” as used herein refers to a process by which a polynucleotide produces a gene product, for example, a RNA or a polypeptide. It includes, without limitation, transcription of the polynucleotide into messenger RNA (mRNA) and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

The terms “identical” or percent “identity” in the context of two or more nucleic acids refer to two or more sequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

One such non-limiting example of a sequence alignment algorithm is the algorithm described in Karlin et al, 1990, Proc. Natl. Acad Sc, 87:2264-2268, as modified in Karlin et al., 1993, Proc. Natl. Acad. Sci., 90:5873-5877, and incorporated into the NBLAST and XBLAST programs (Altschul et al., 1991, Nucleic Acids Res., 25:3389-3402). In certain embodiments. Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, Calif.) or Megalign (DNASTAR) are additional publicly available software programs that can be used to align sequences. In certain embodiments, the percent identity between two nucleotide sequences is determined using the GAP program in the GCG software package (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch (J. Mol. Biol. (48):444-453 (1970)) can be used to determine the percent identity between two amino acid sequences (e.g., using either a BLOSUM 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. One skilled in the art can determine appropriate parameters for maximal alignment by particular alignment software. In certain embodiments, the default parameters of the alignment software are used.

In certain embodiments, the percentage identity “X” of a first nucleotide sequence to a second nucleotide sequence is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

Different regions within a single polynucleotide target sequence that align with a polynucleotide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

As used herein, the terms “homologous” and “homology” are interchangeable with the terms “identity” and “identical.”

The term “naturally occurring variant thereof” refers to variants of the CAMK2D polypeptide sequence or CAMK2D nucleic acid sequence (e.g., transcript) which exist naturally within the defined taxonomic group, such as mammalian, such as mouse, monkey, and human. Typically, when referring to “naturally occurring variants” of a polynucleotide the term also can encompass any allelic variant of the CAMK2D-encoding genomic DNA which is found at Chromosomal position 4q26 (i.e., residues 113,451,032 to 113,761,927 of GenBank Accession No. NC_000004.12) by chromosomal translocation or duplication, and the RNA, such as mRNA derived therefrom. “Naturally occurring variants” can also include variants derived from alternative splicing of the CAMK2D mRNA. When referenced to a specific polypeptide sequence, e.g., the term also includes naturally occurring forms of the protein, which can therefore be processed, e.g., by co- or post-translational modifications, such as signal peptide cleavage, proteolytic cleavage, glycosylation, etc.

In determining the degree of “complementarity” between the ASOs of the disclosure (or regions thereof) and the target region of the nucleic acid which encodes mammalian CAMK2D (e.g., the CAMK2D gene), such as those disclosed herein, the degree of “complementarity” (also, “homology” or “identity”) is expressed as the percentage identity (or percentage homology) between the sequence of the ASO (or region thereof) and the sequence of the target region (or the reverse complement of the target region) that best aligns therewith. The percentage is calculated by counting the number of aligned bases that are identical between the two sequences, dividing by the total number of contiguous monomers in the ASO, and multiplying by 100. In such a comparison, if gaps exist, it is preferable that such gaps are merely mismatches rather than areas where the number of monomers within the gap differs between the ASO of the disclosure and the target region.

The term “complement” as used herein indicates a sequence that is complementary to a reference sequence. It is well known that complementarity is the base principle of DNA replication and transcription as it is a property shared between two DNA or RNA sequences, such that when they arm aligned antiparallel to each other, the nucleotide bases at each position in the sequences will be complementary, much like looking in the mirror and seeing the reverse of things. Therefore, for example, the complement of a sequence of 5′“ATGC”3′ can be written as 3′“TACG”5′ or 5′“GCAT”3′. The terms “reverse complement”, “reverse complementary”, and “reverse complementarity” as used herein are interchangeable with the terms “complement”, “complementary”, and “complementarity.” In some embodiments, the term “complementary” refers to 100% match or complementarity (i.e., fully complementary) to a contiguous nucleic acid sequence within a CAMK2D transcript. In some embodiments, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a contiguous nucleic acid sequence within a CAMK2 D transcript.

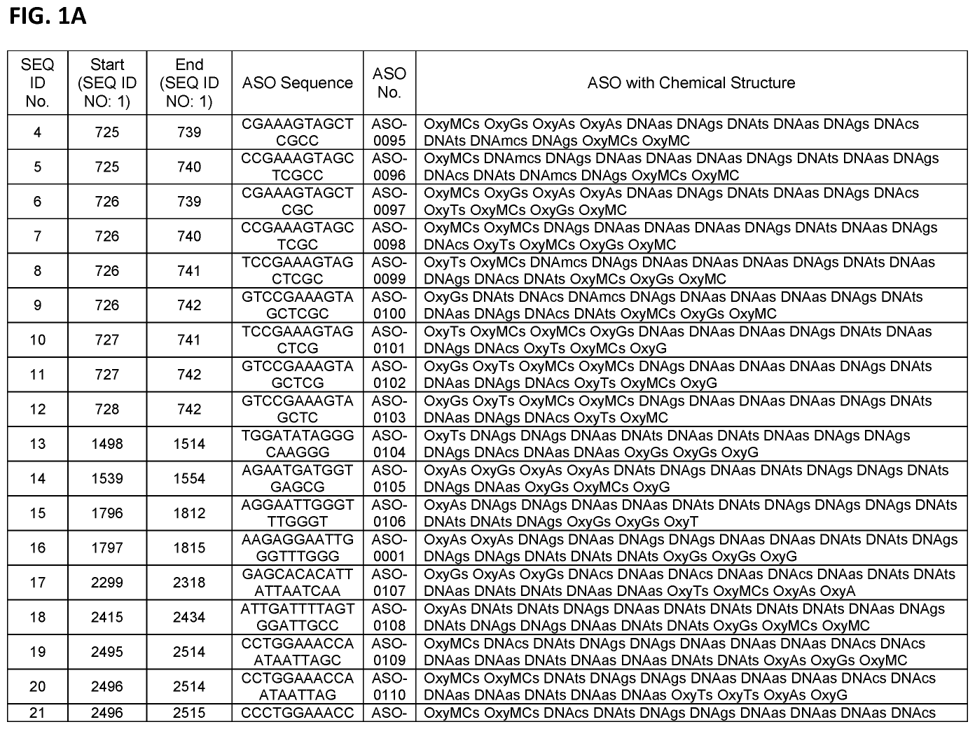

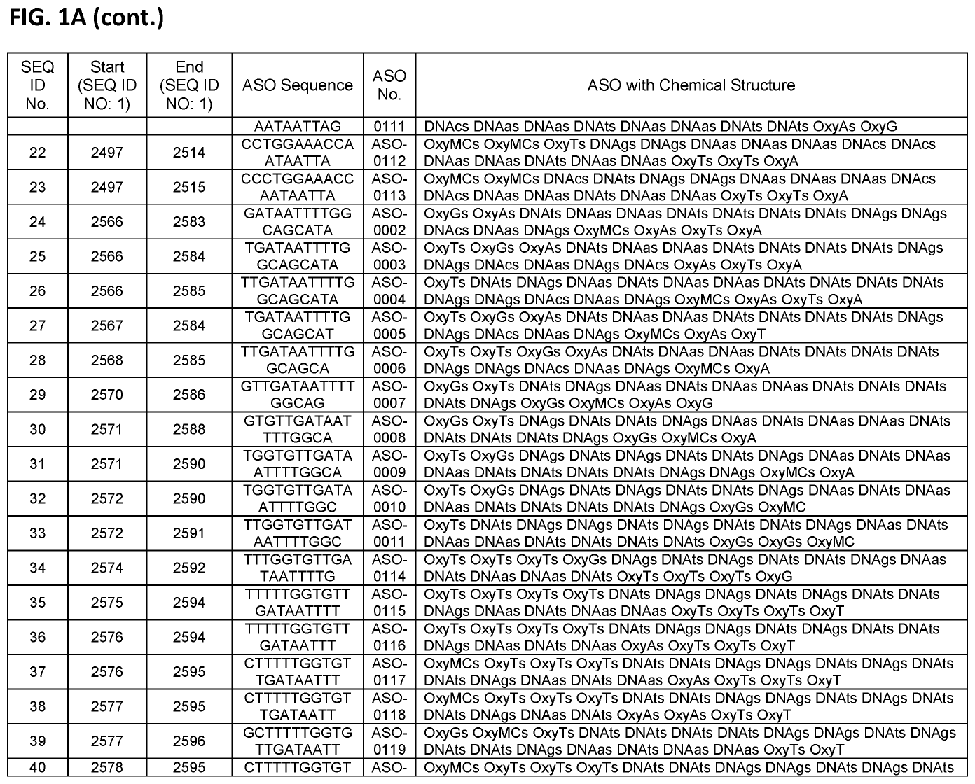

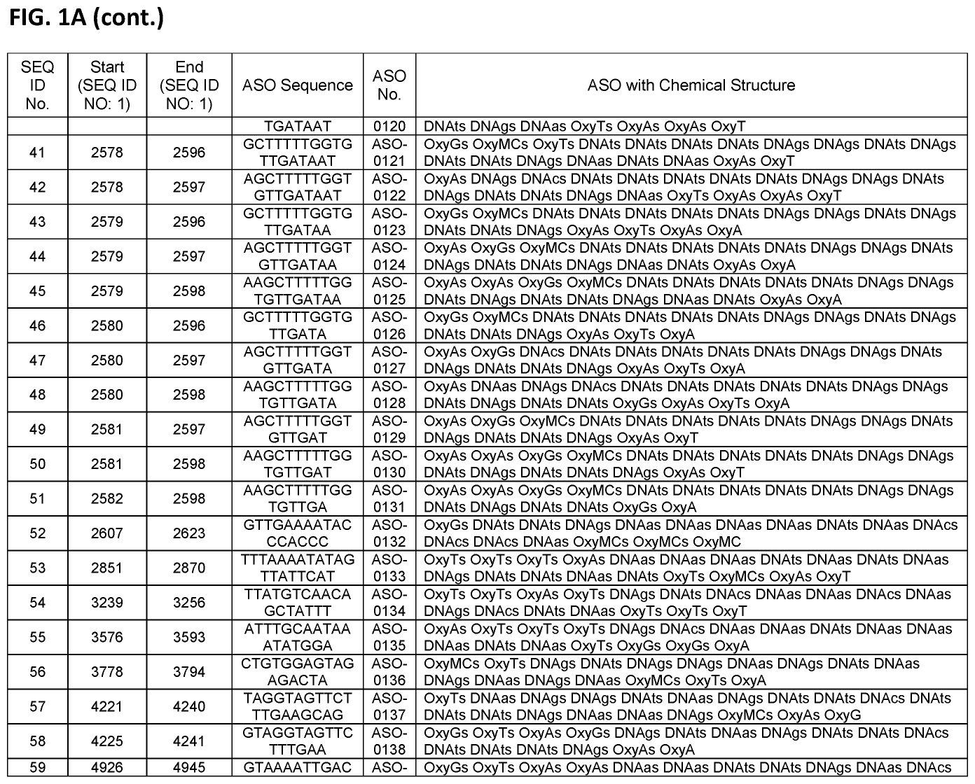

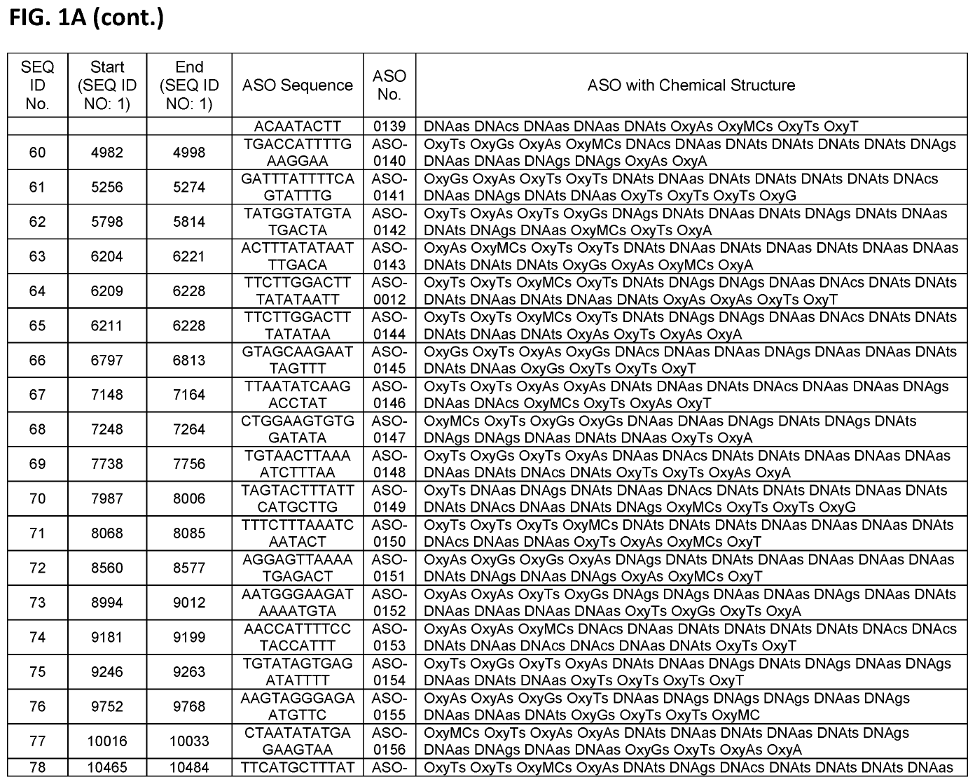

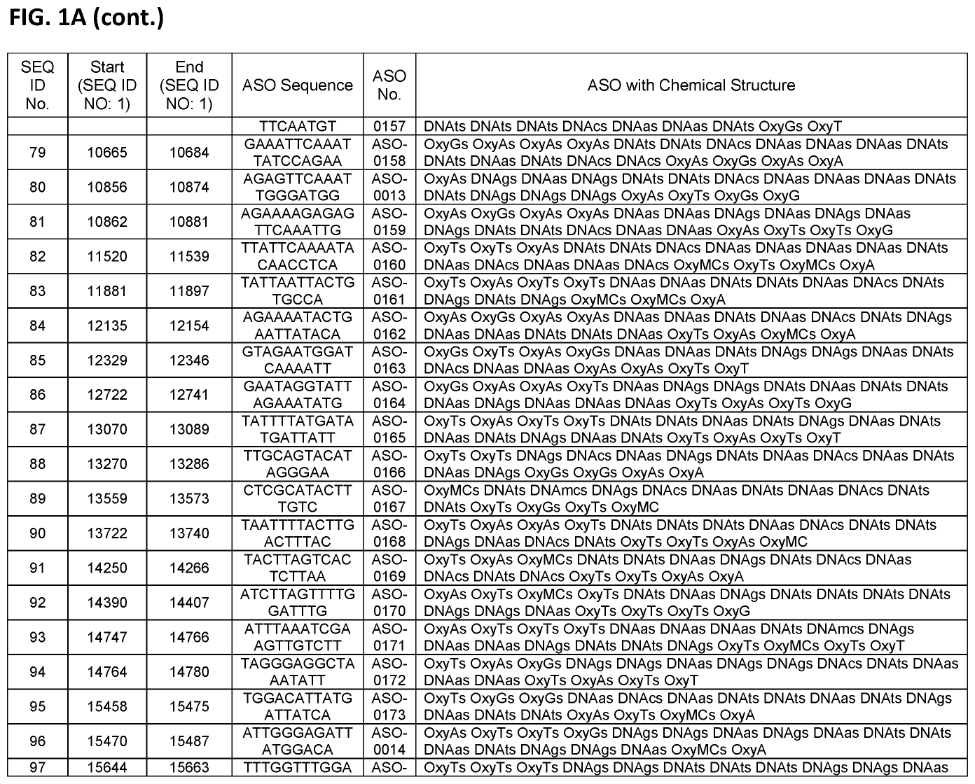

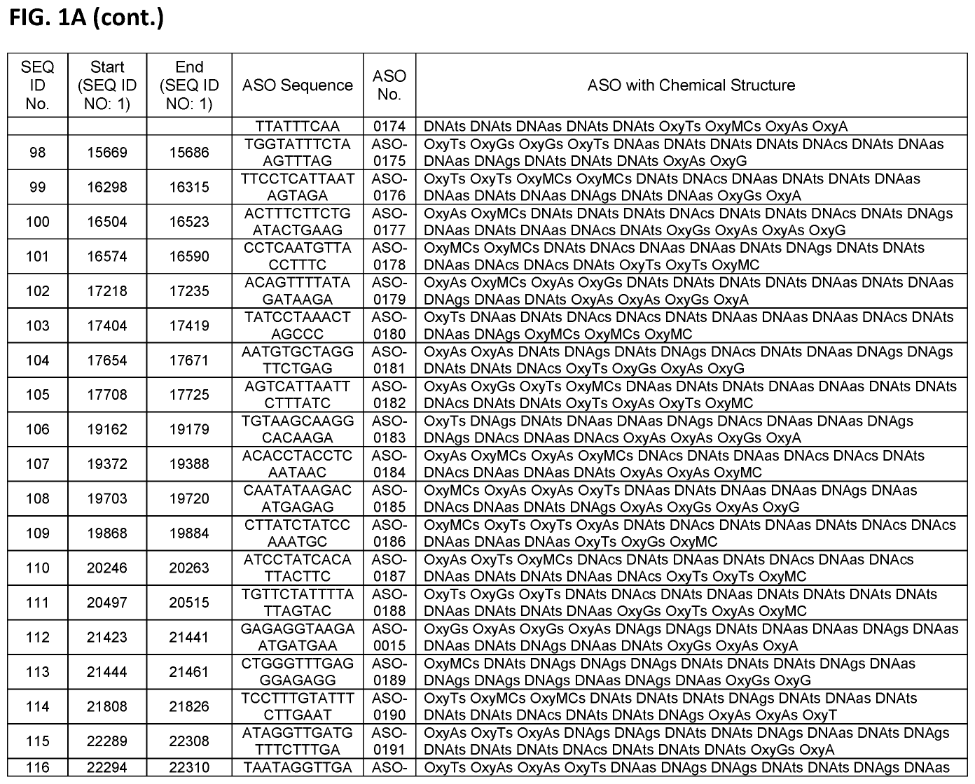

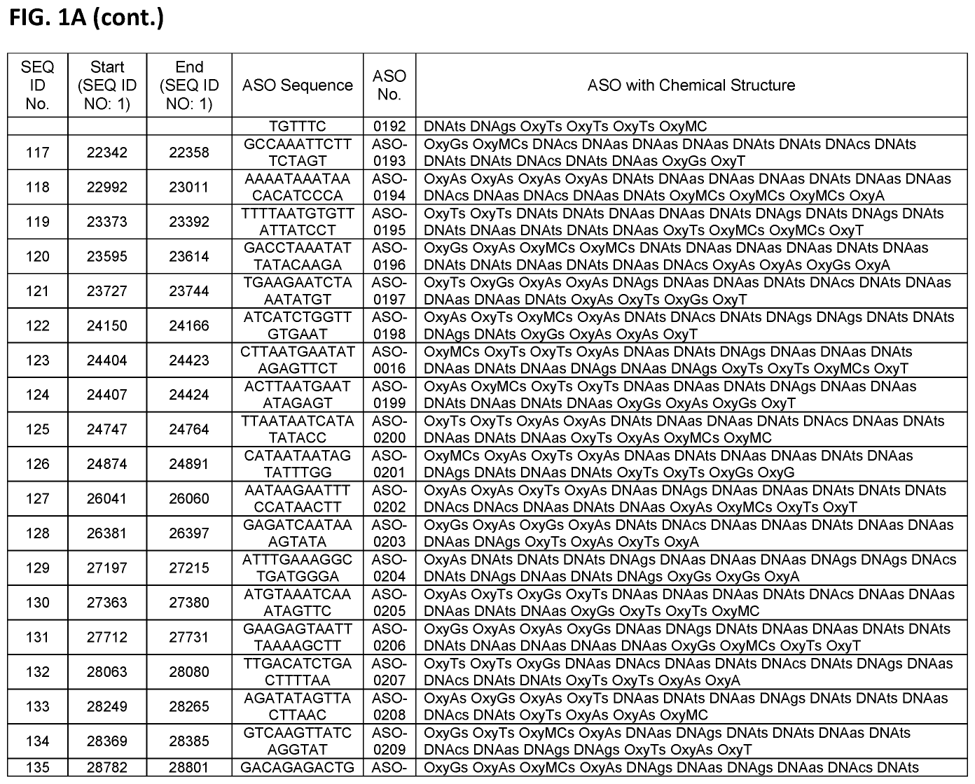

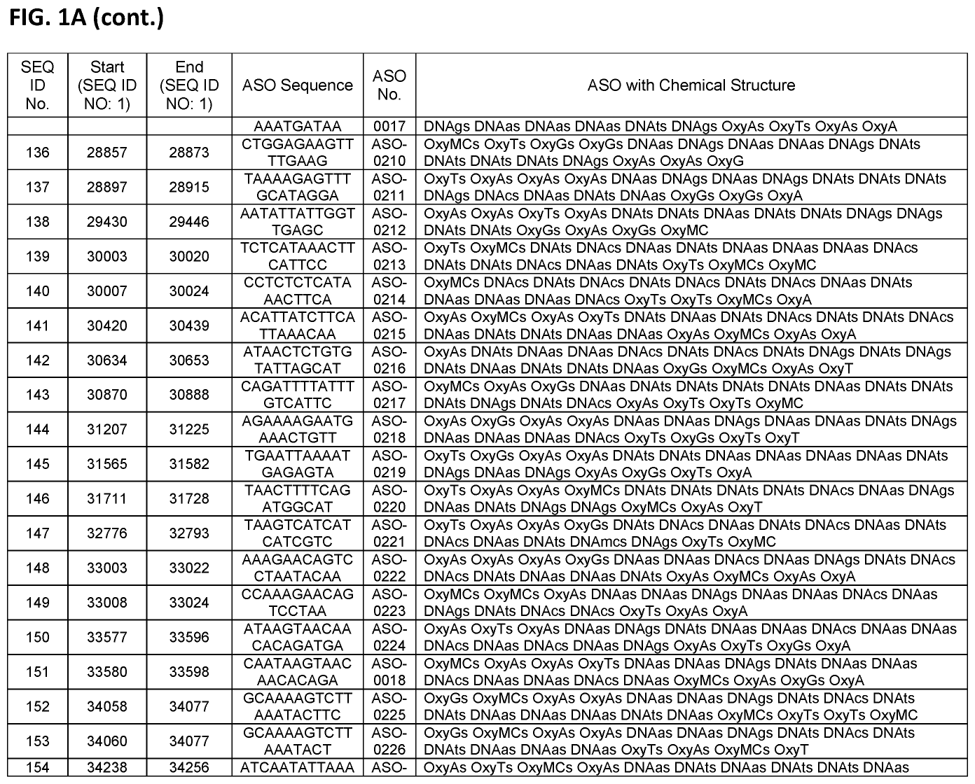

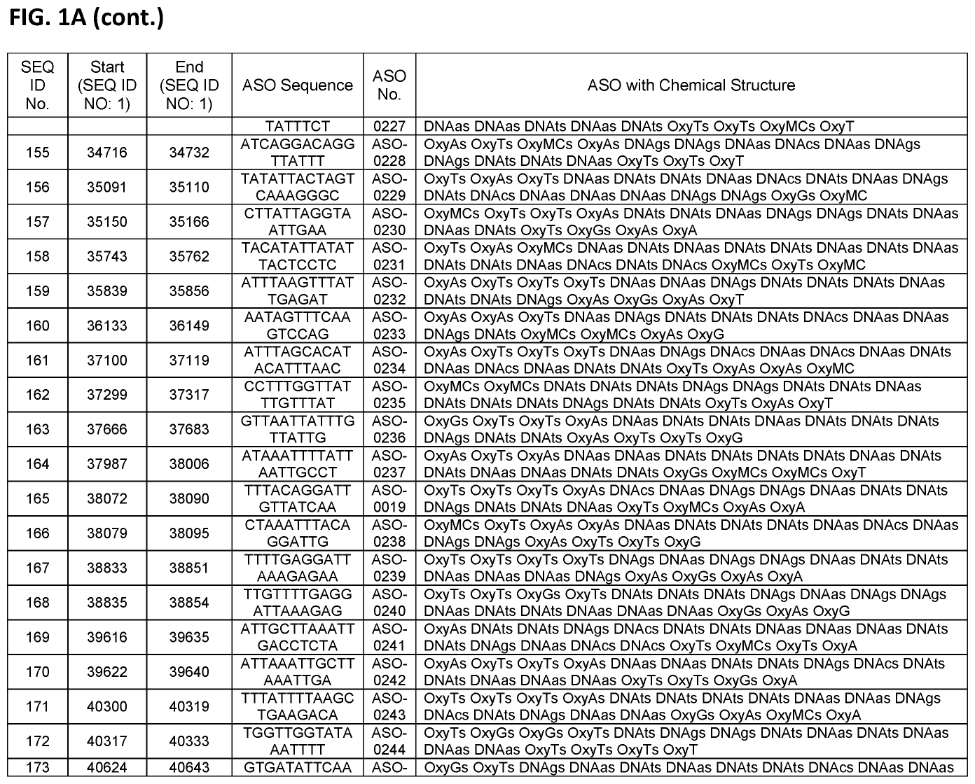

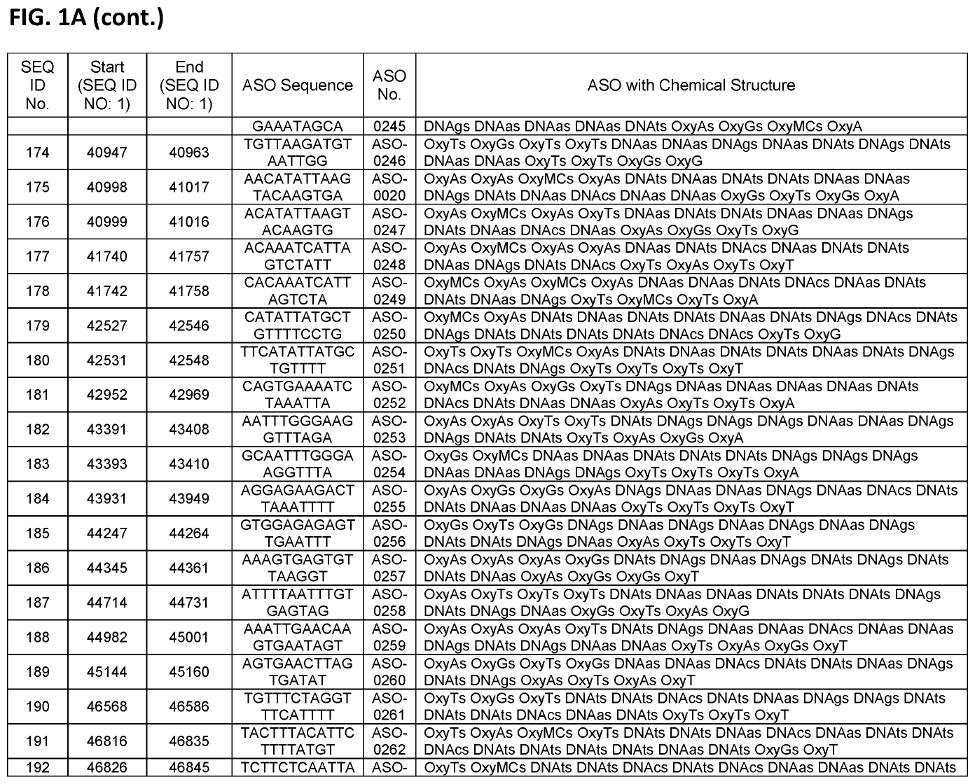

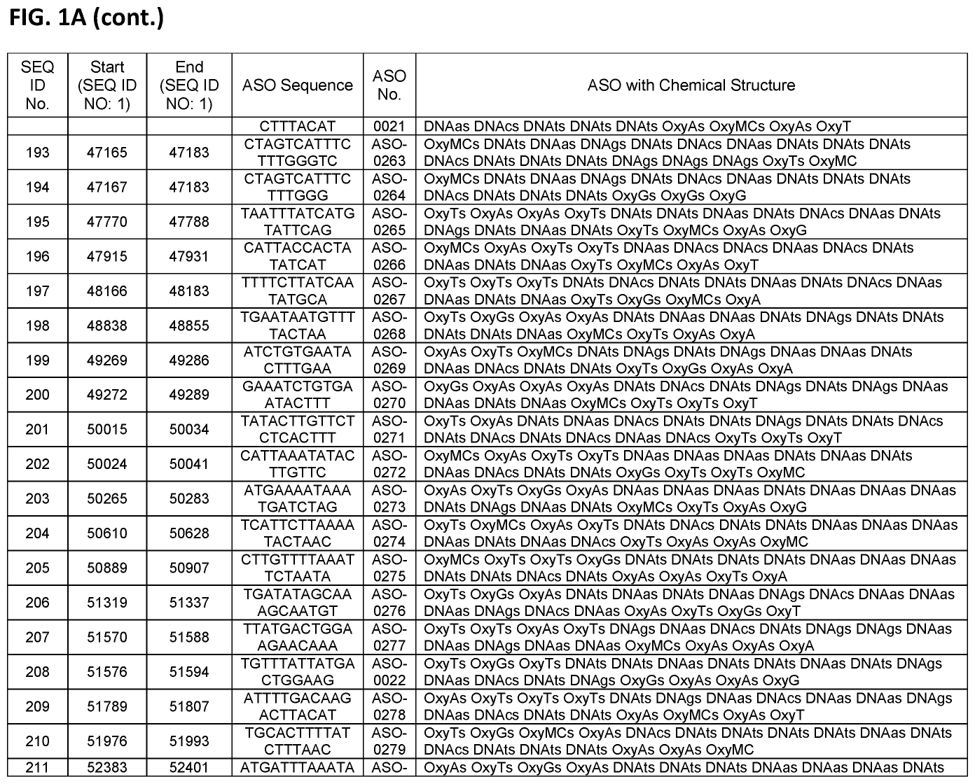

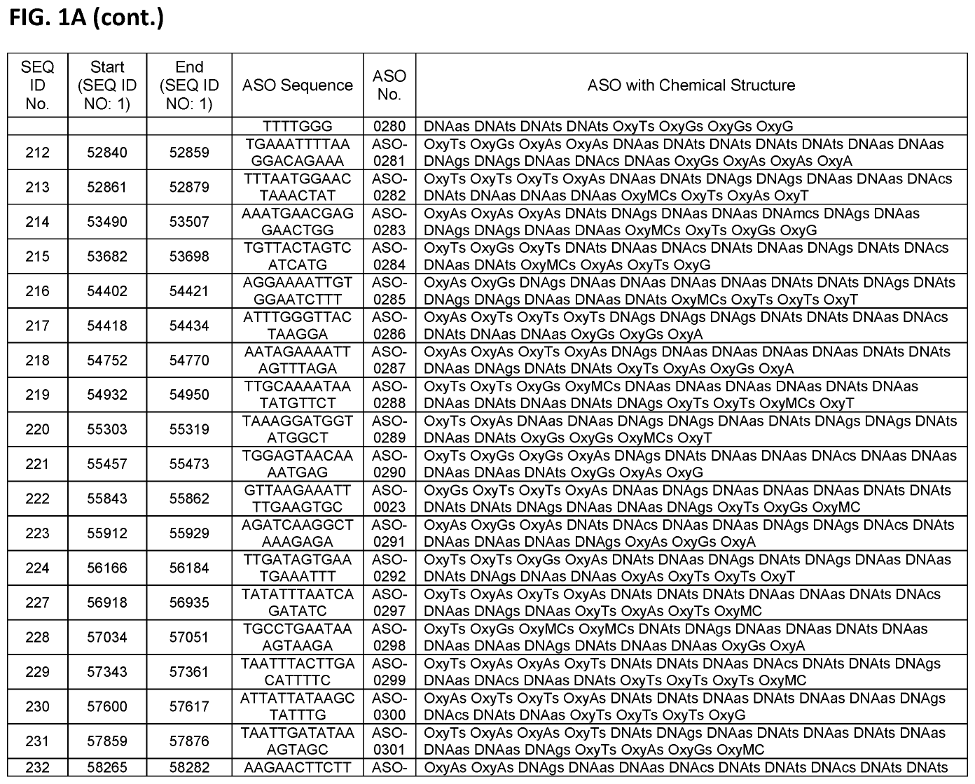

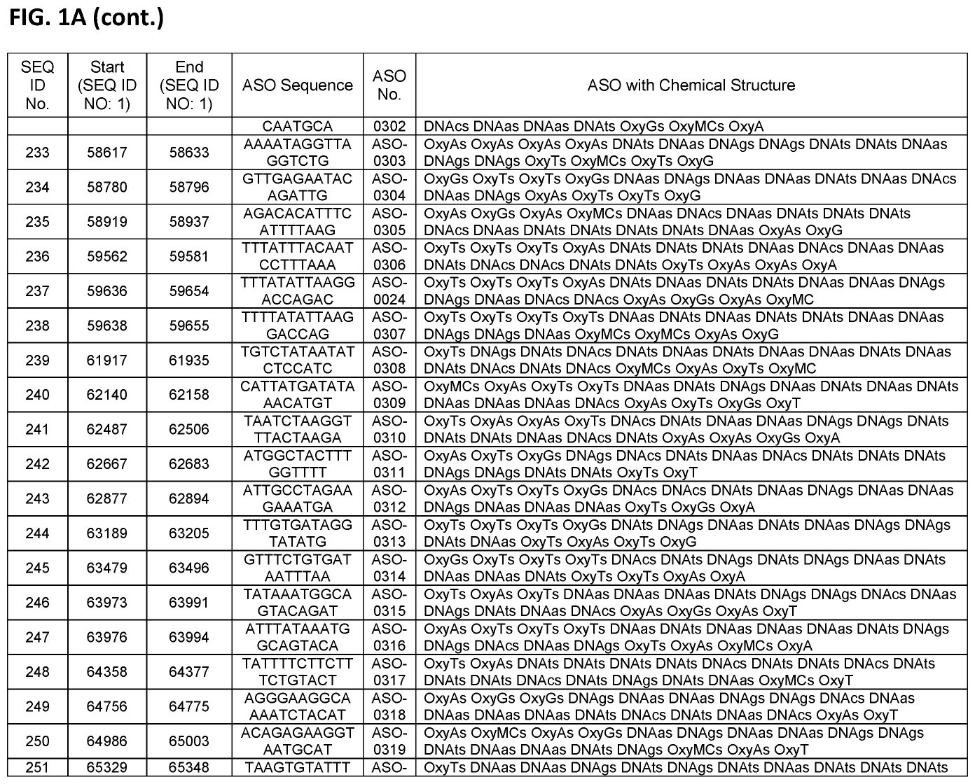

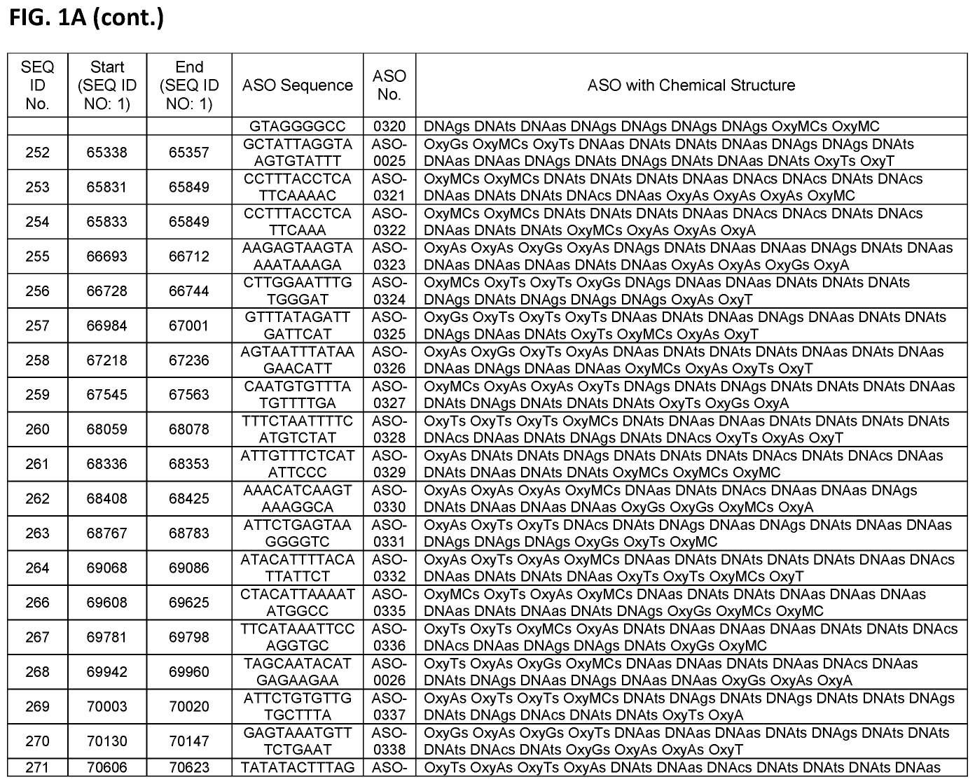

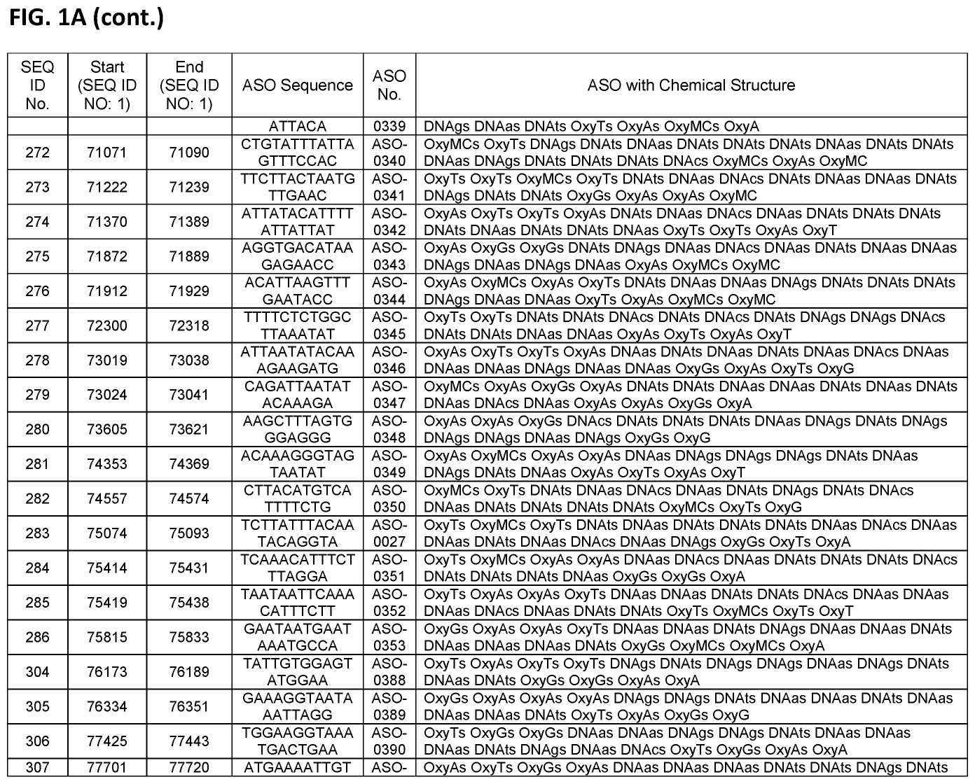

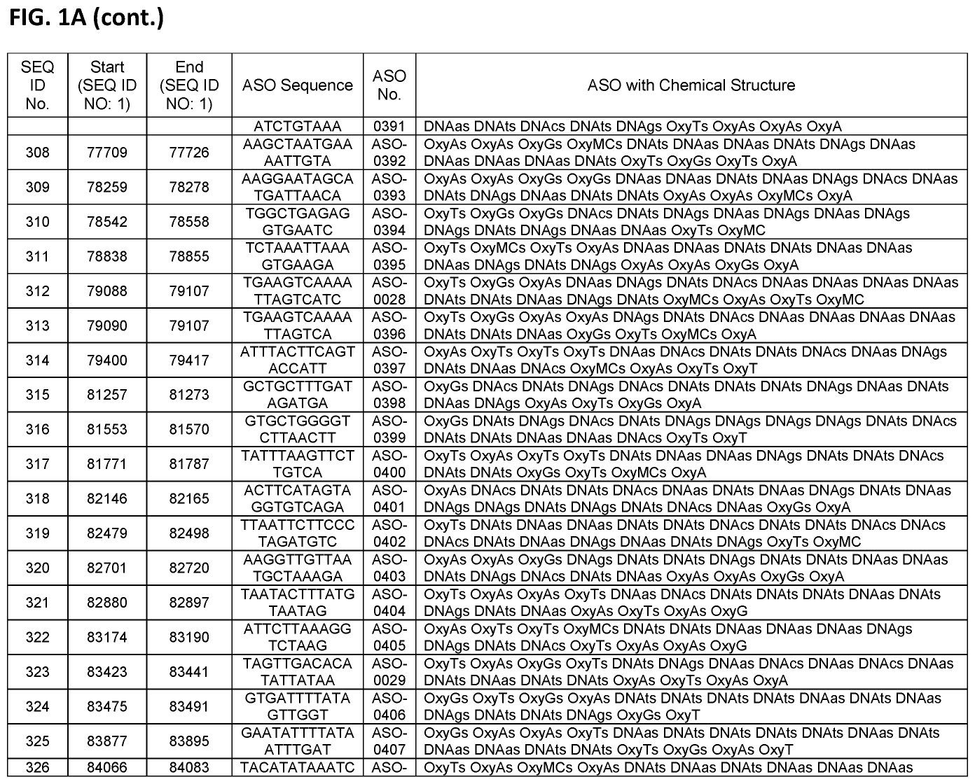

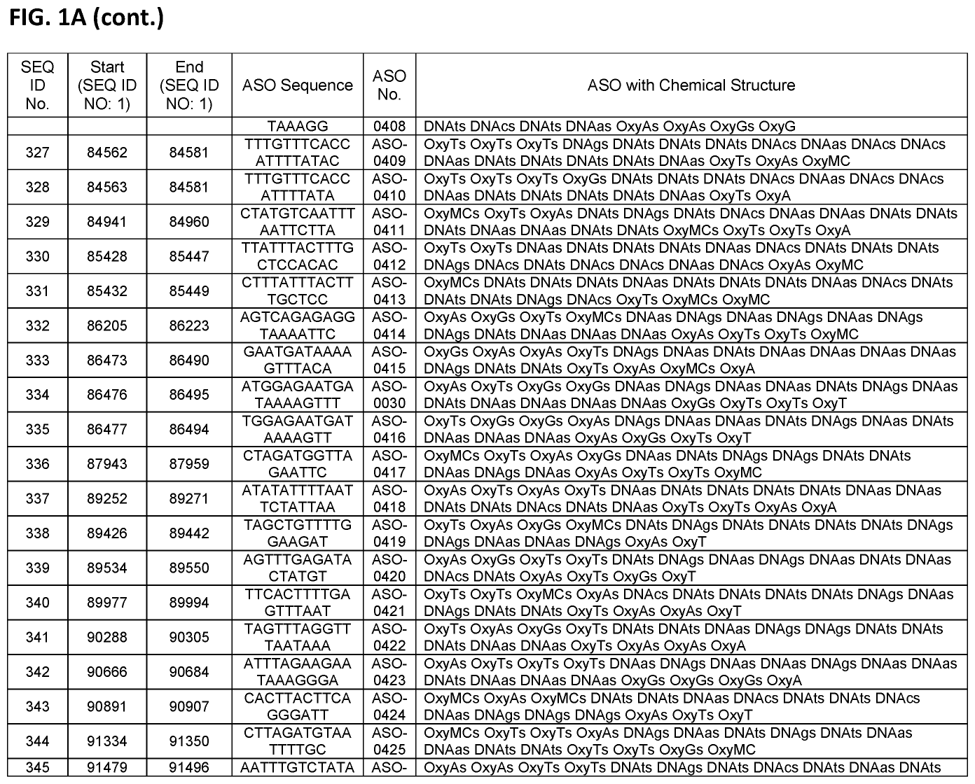

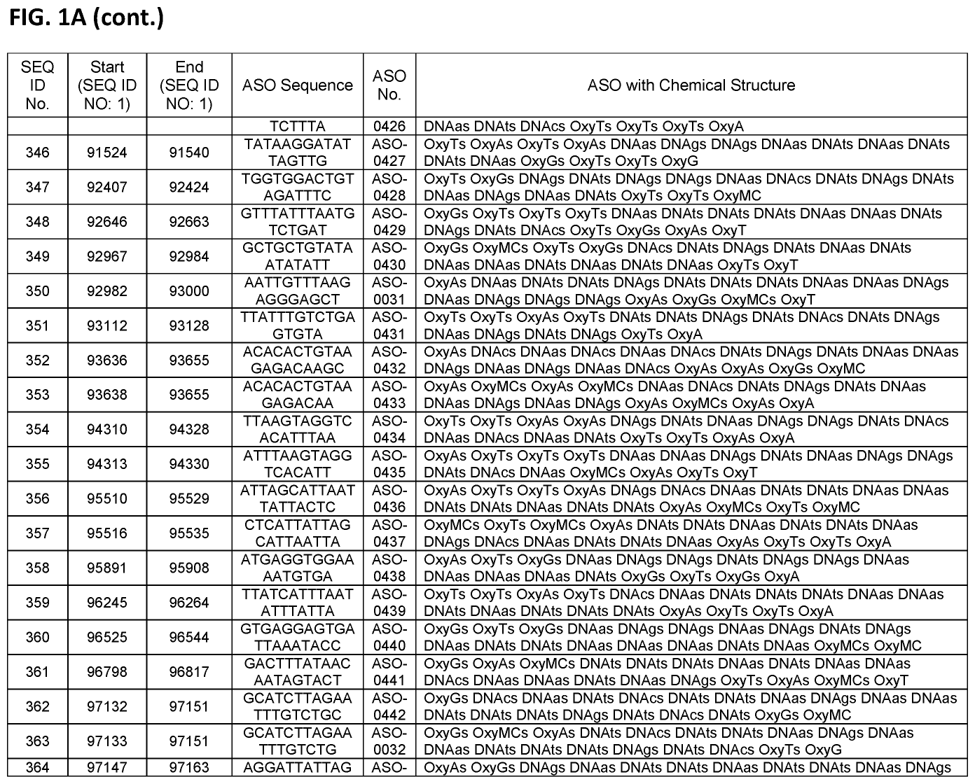

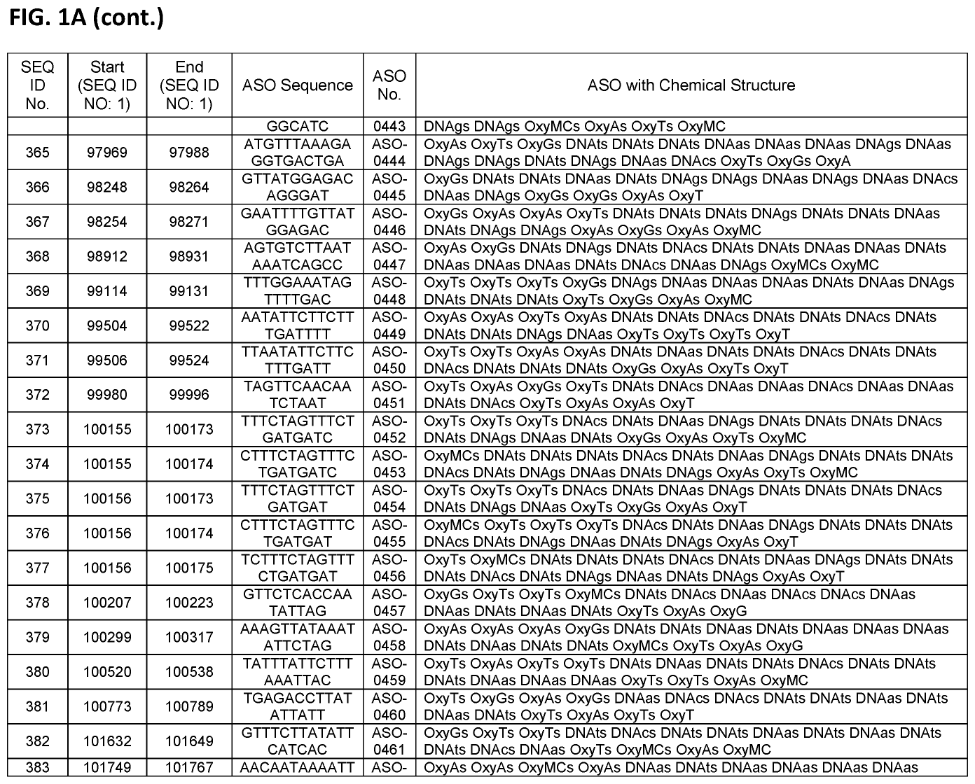

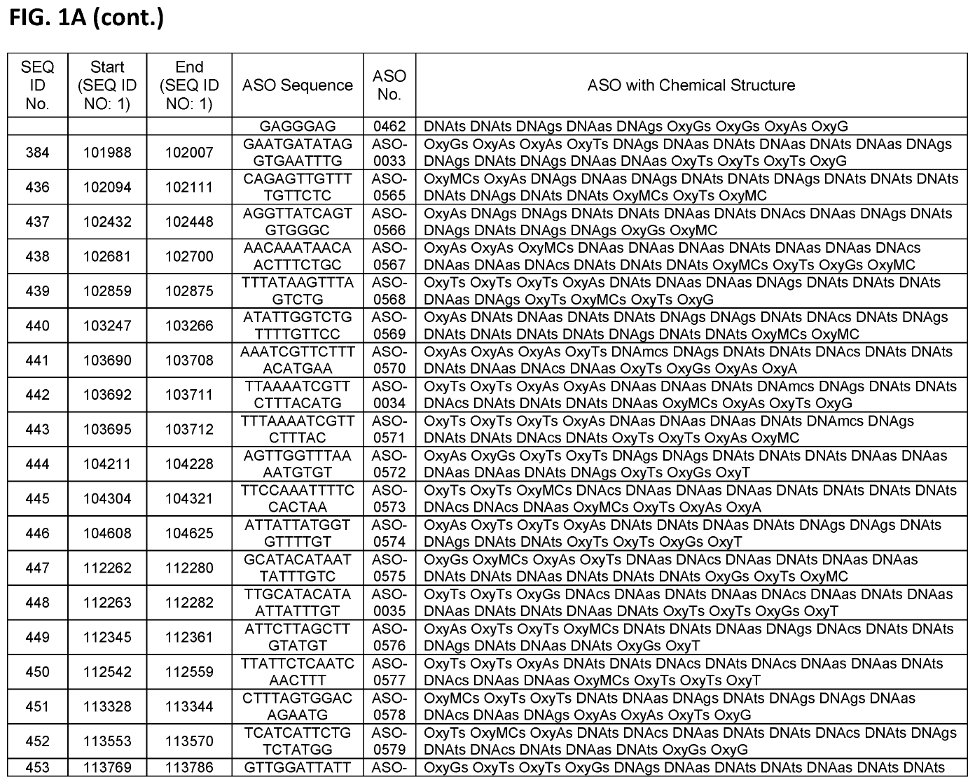

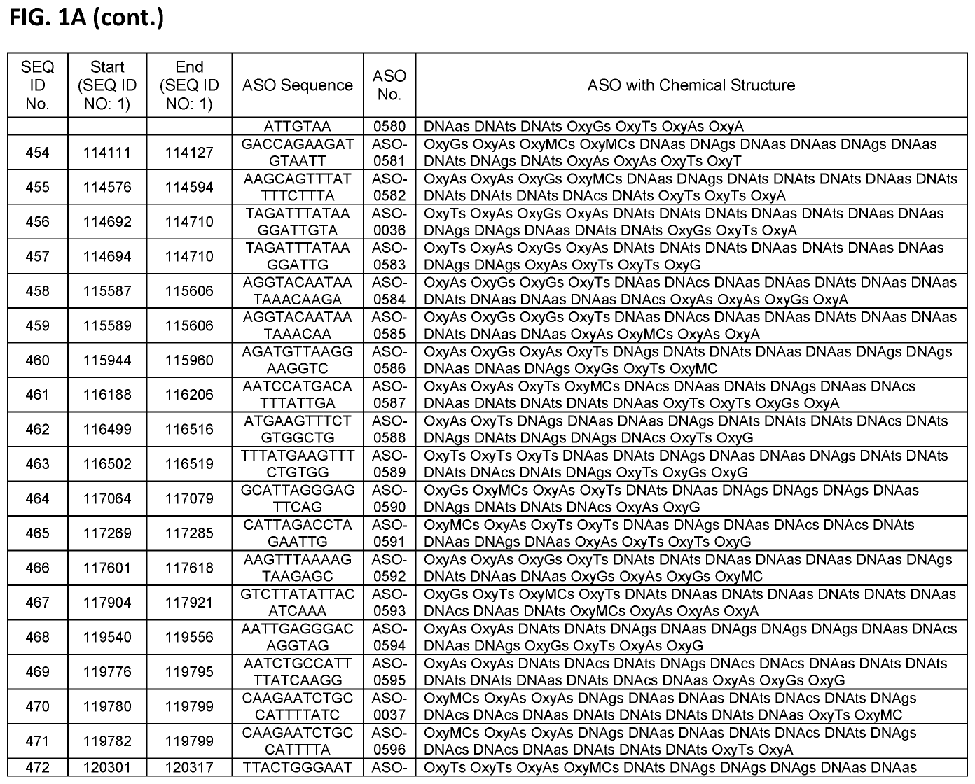

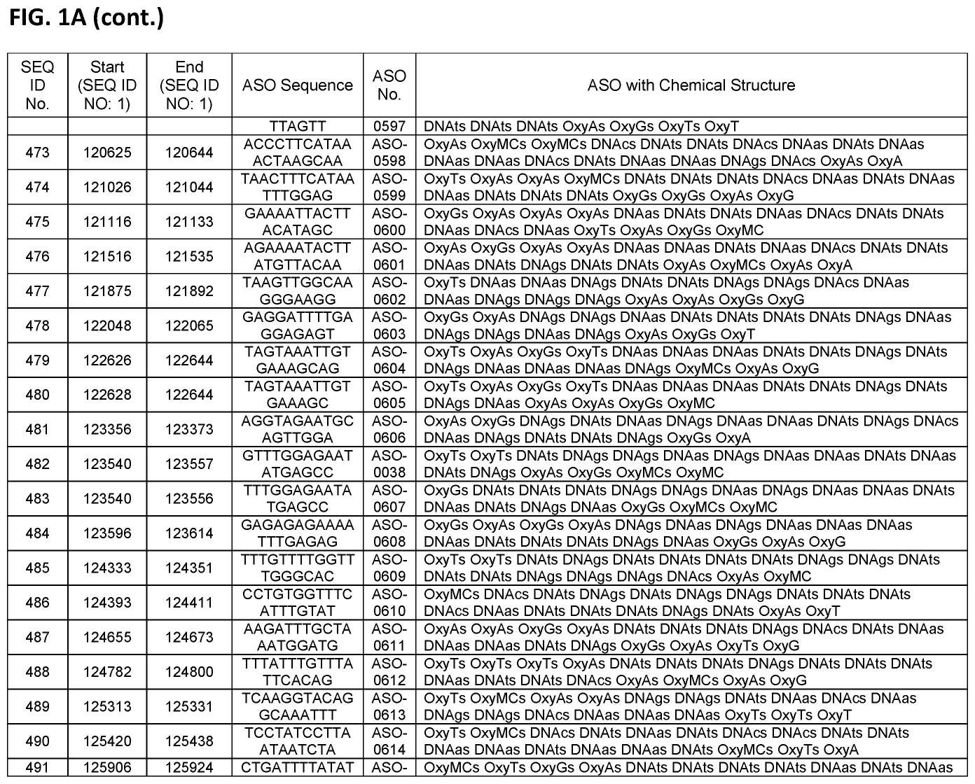

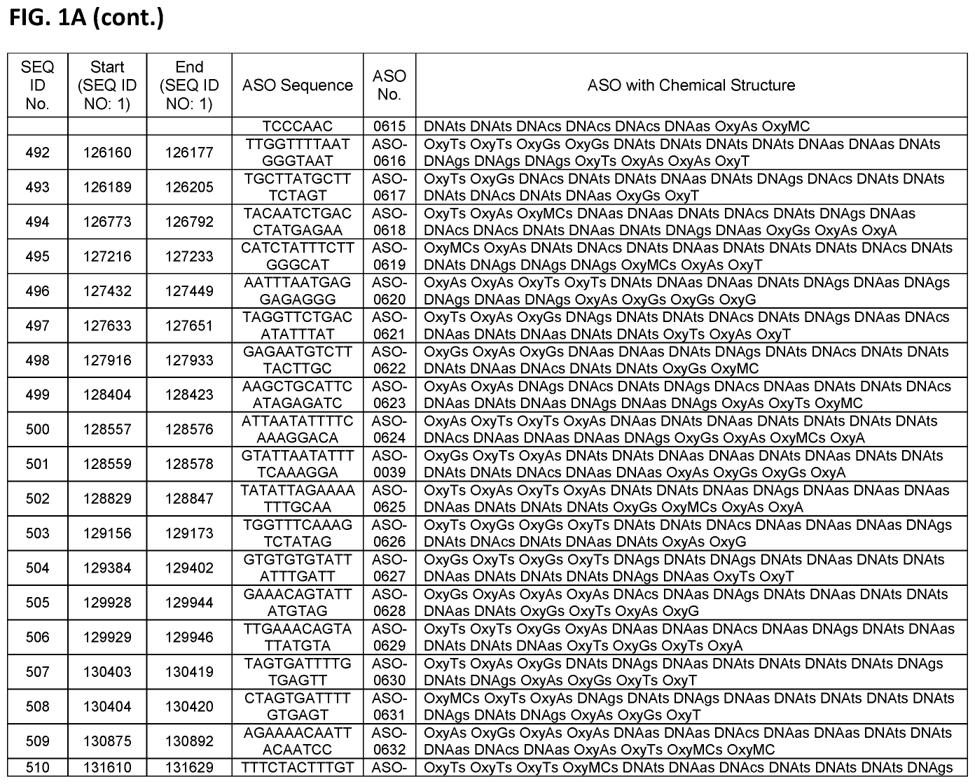

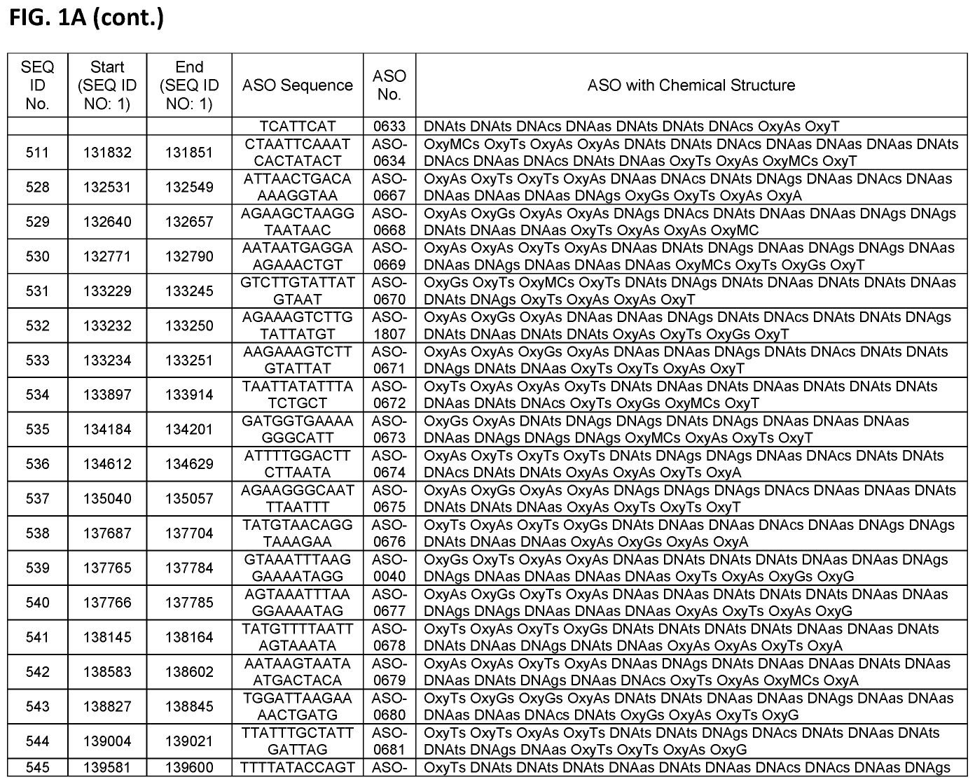

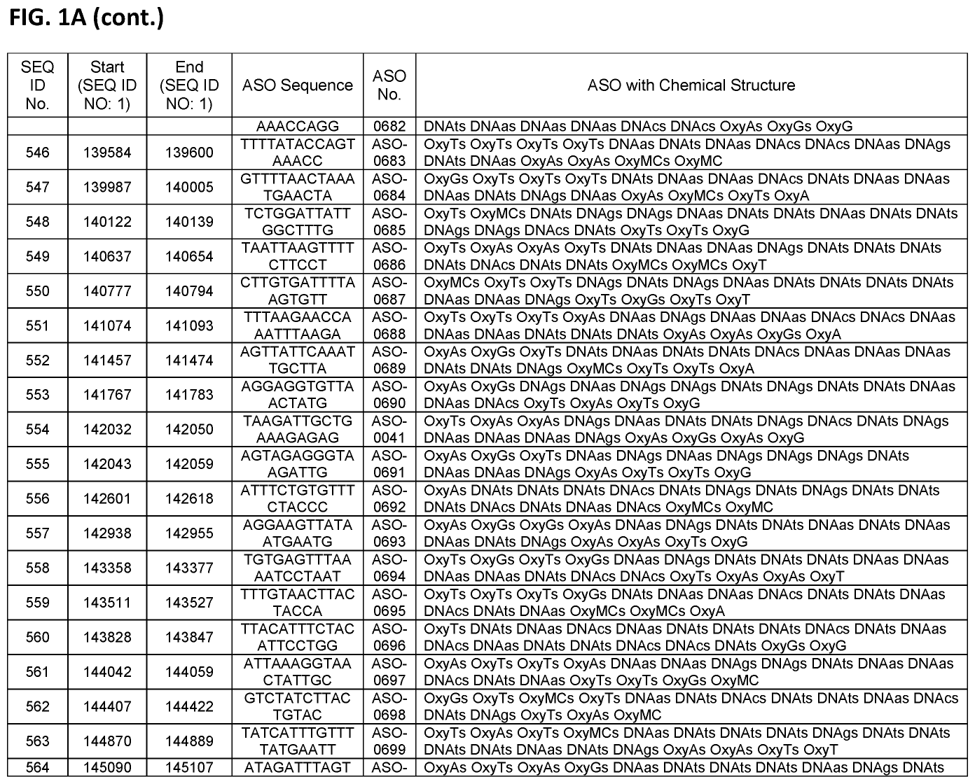

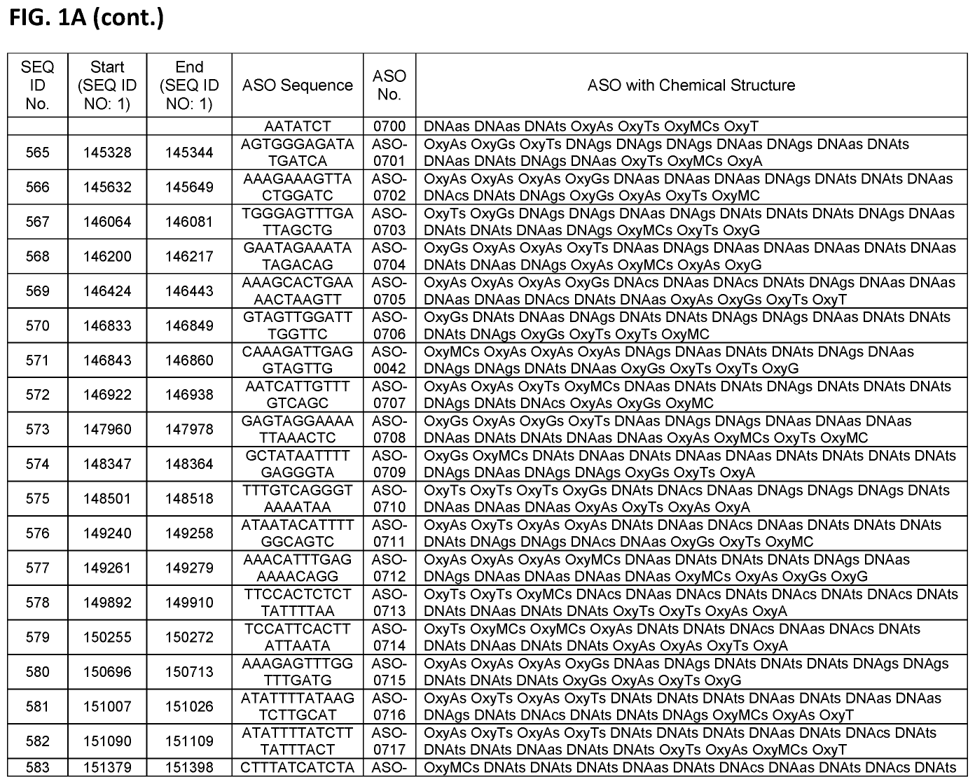

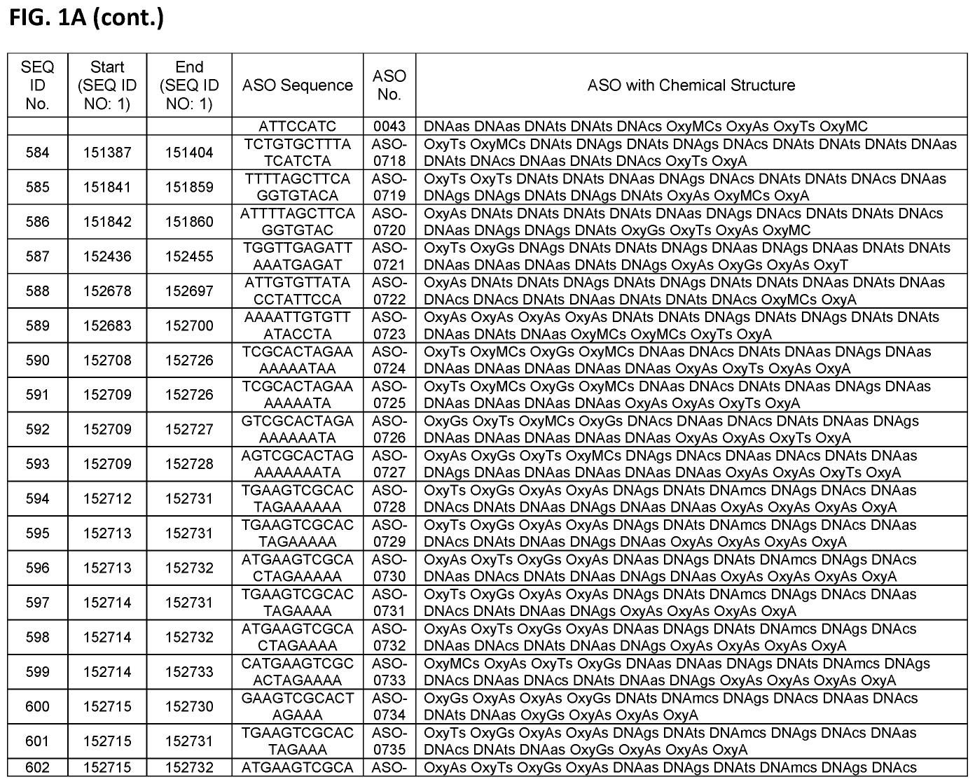

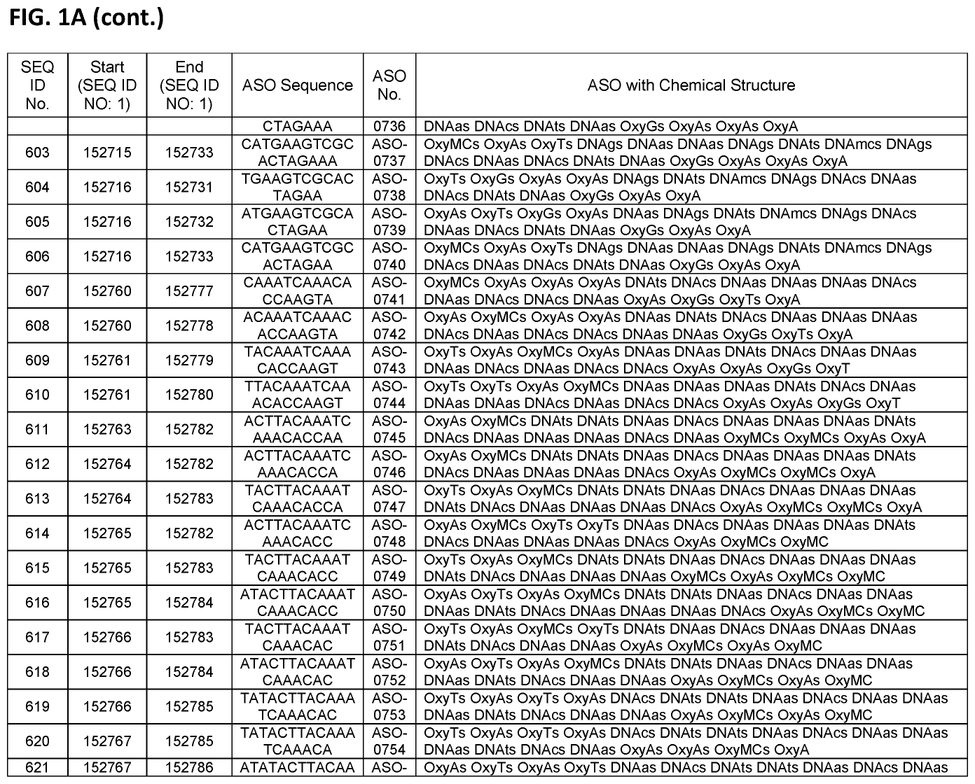

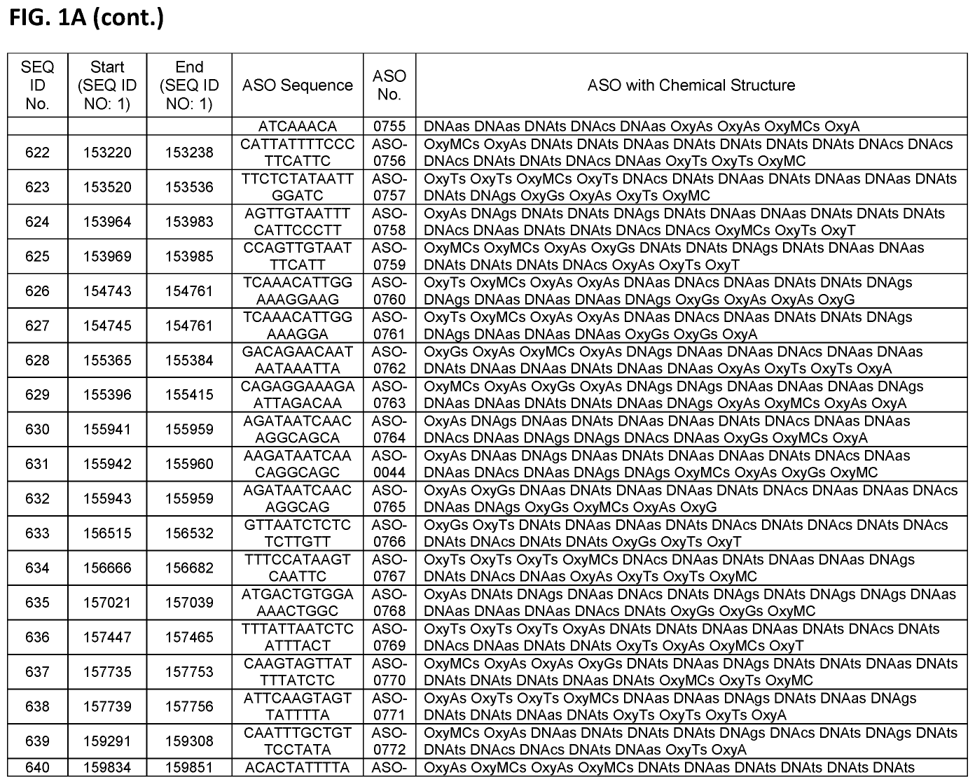

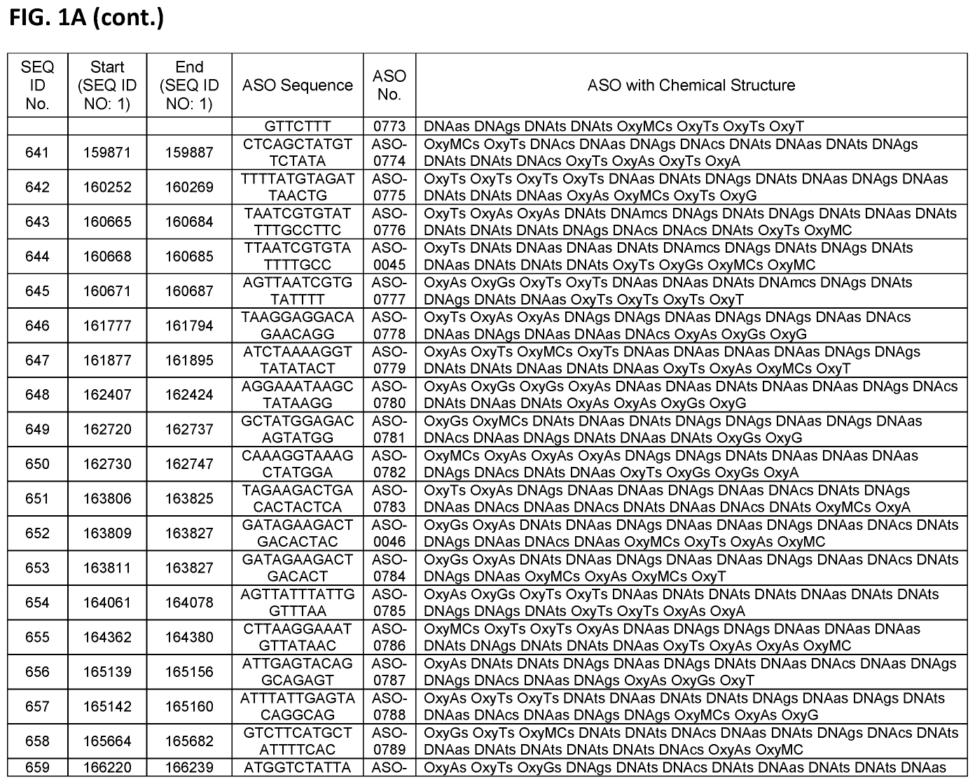

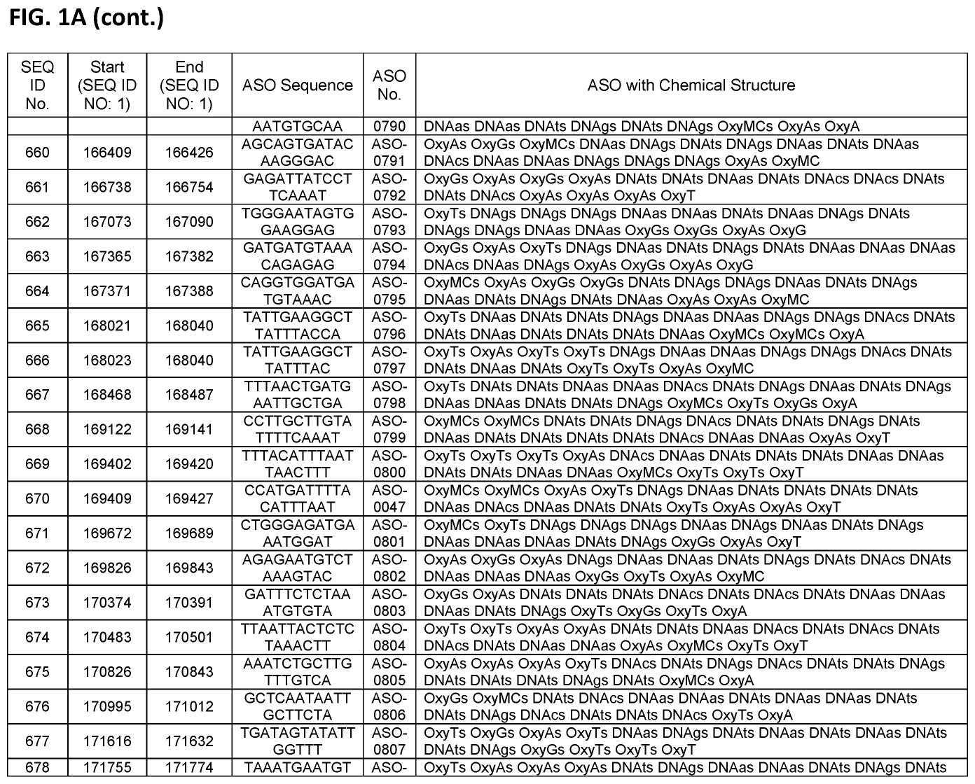

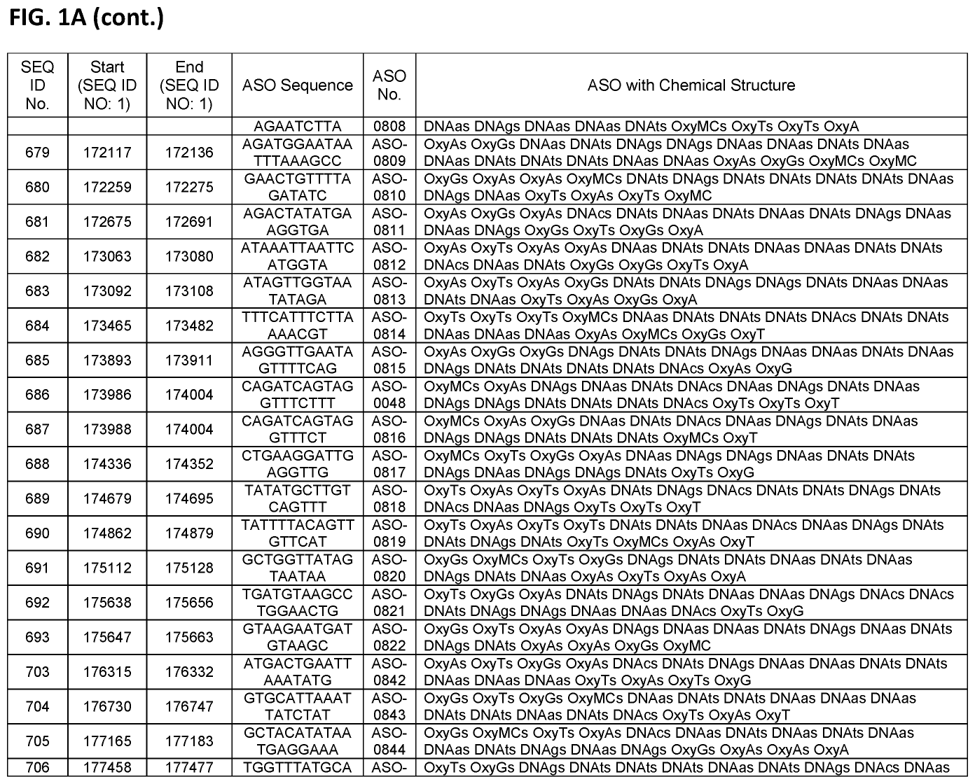

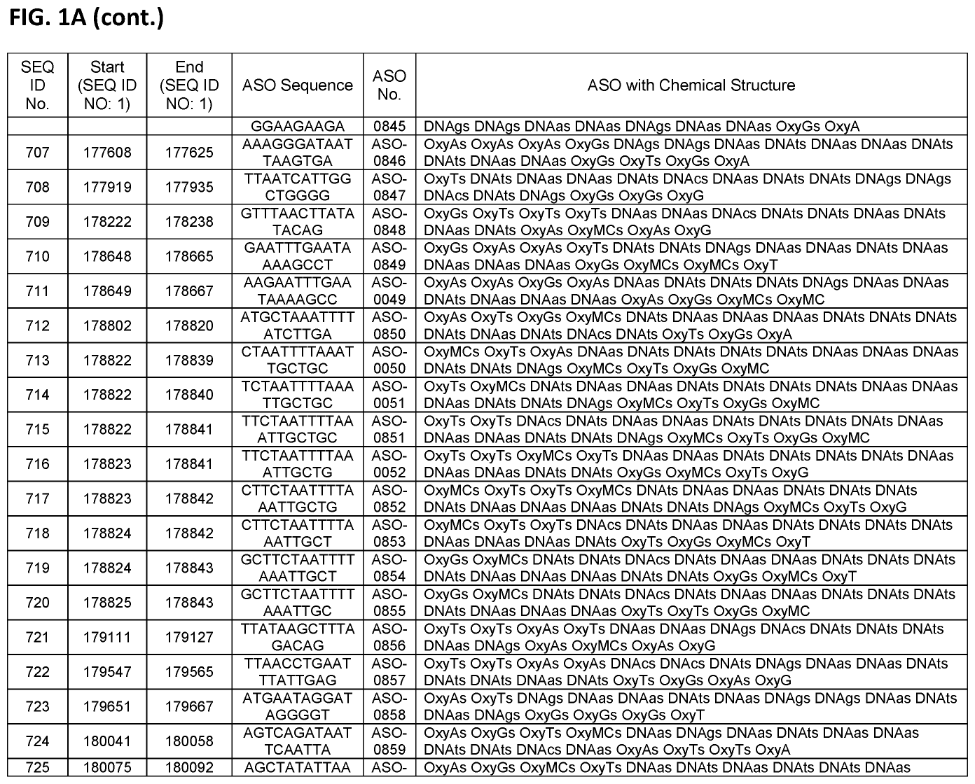

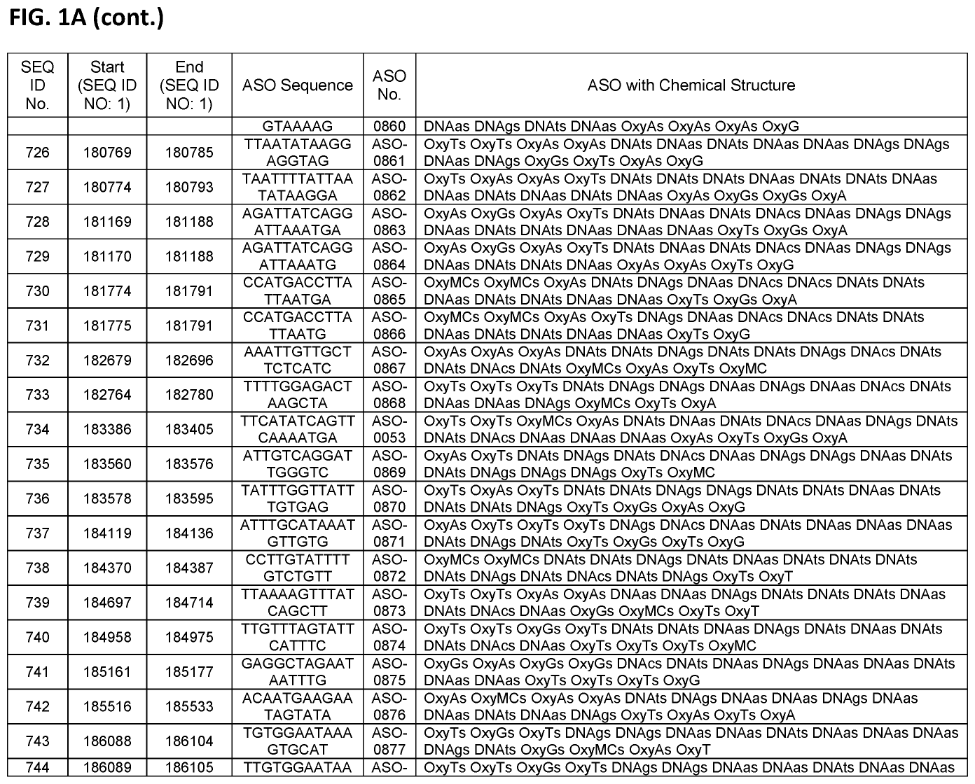

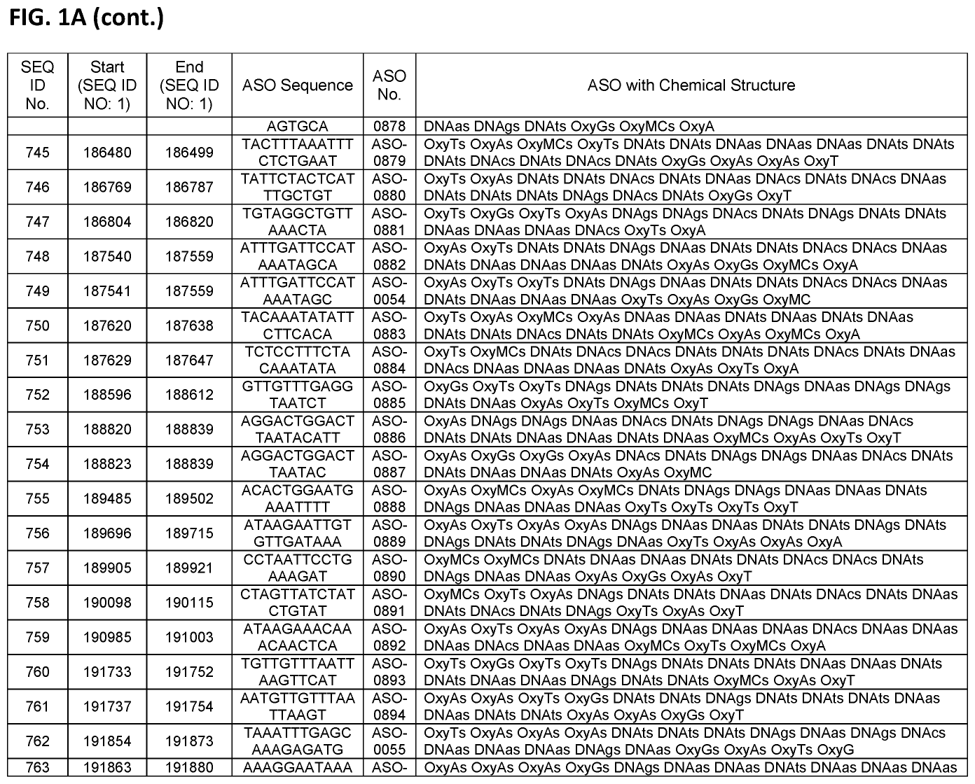

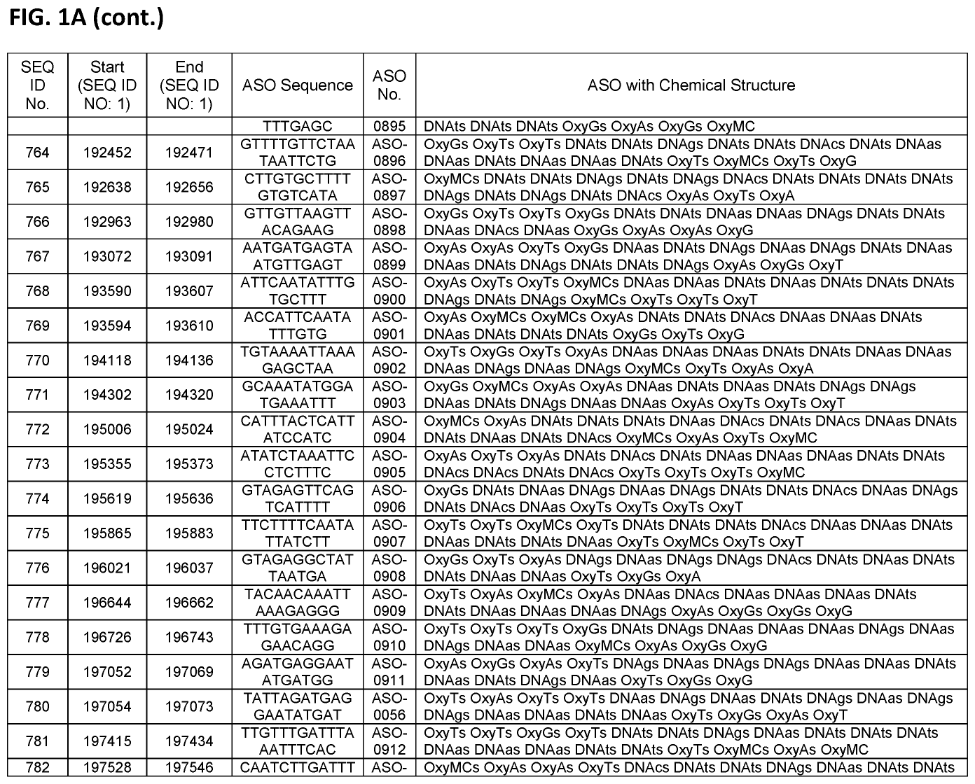

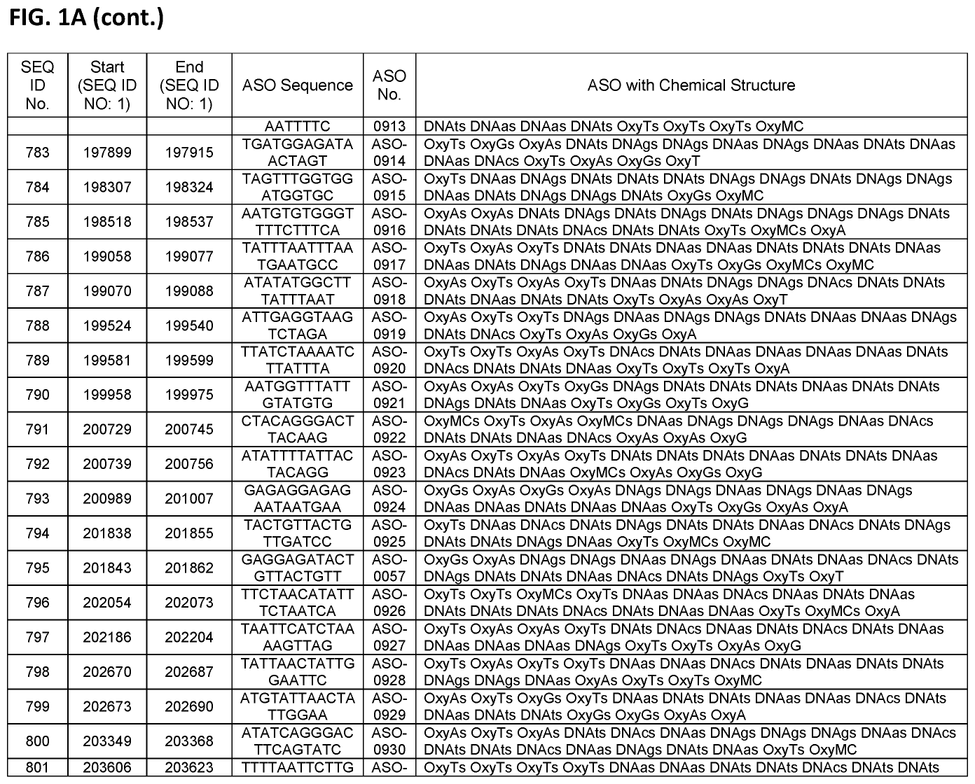

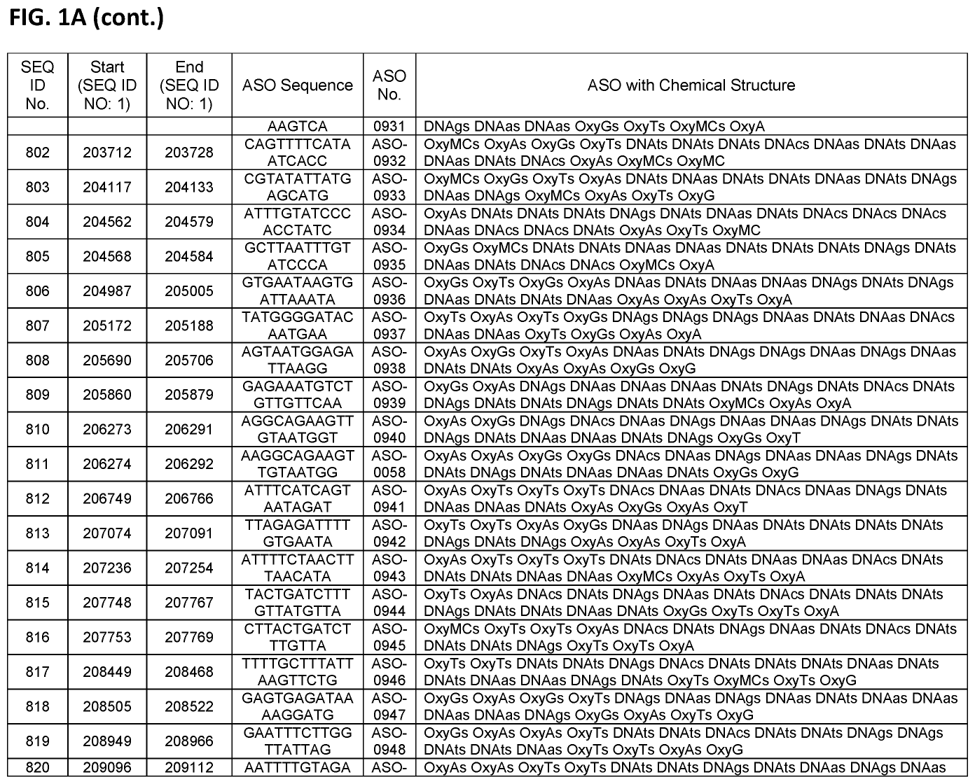

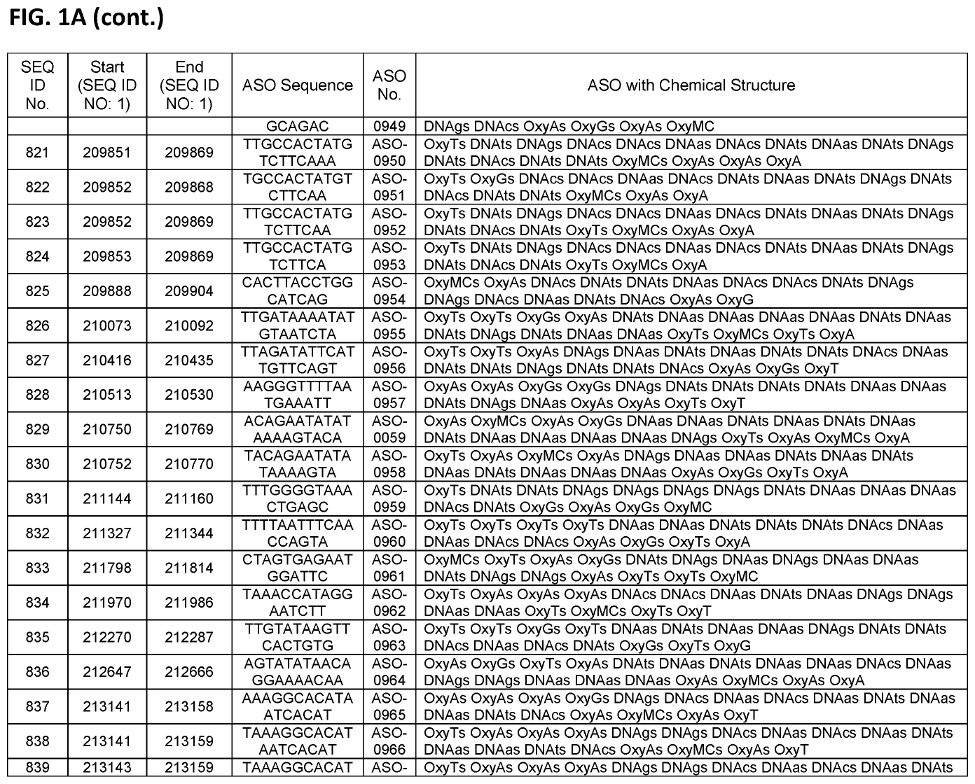

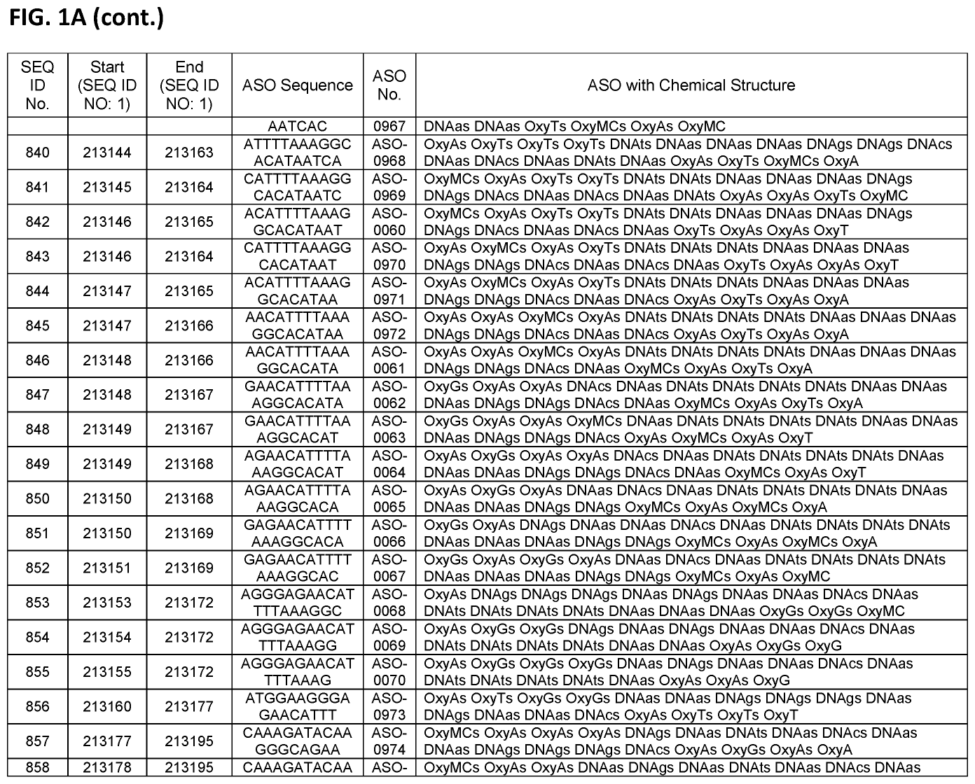

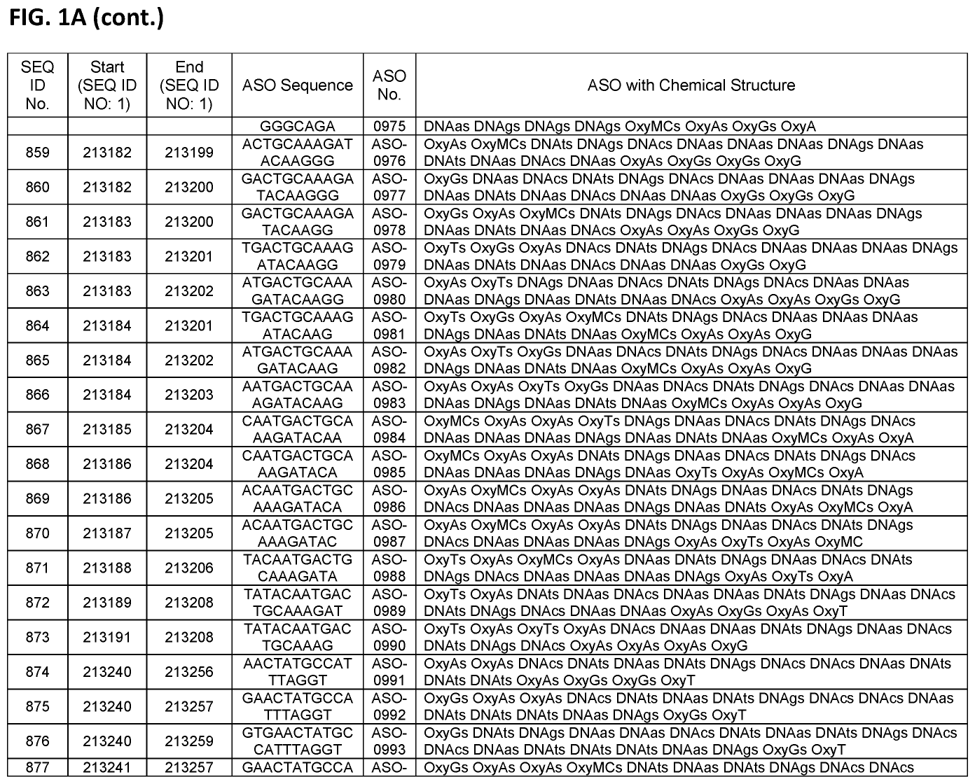

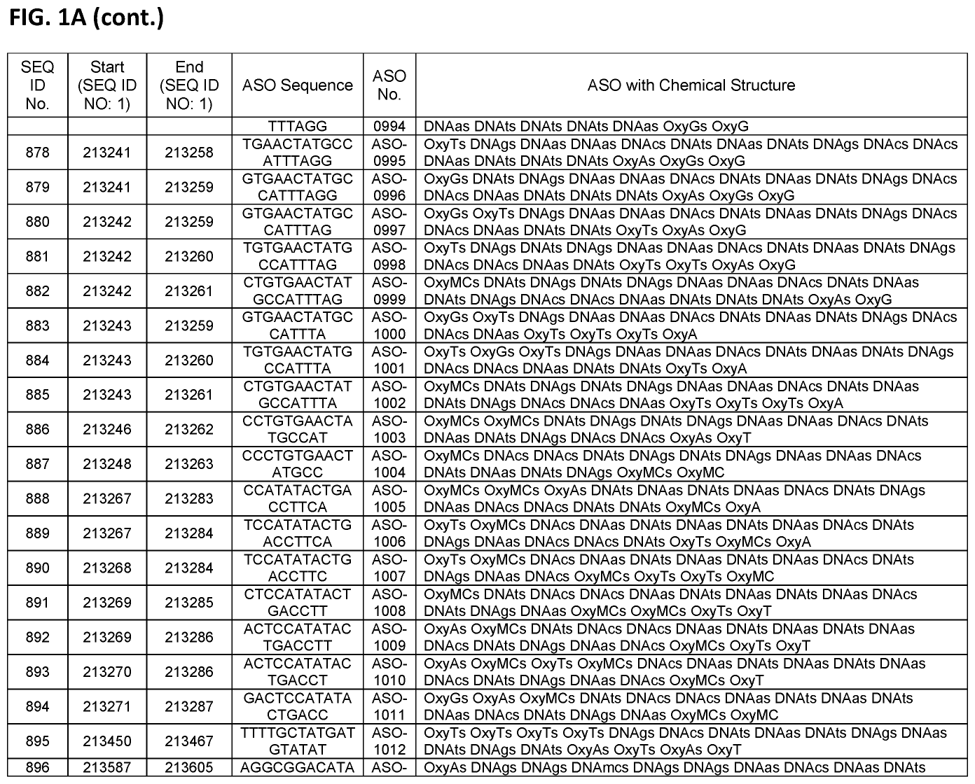

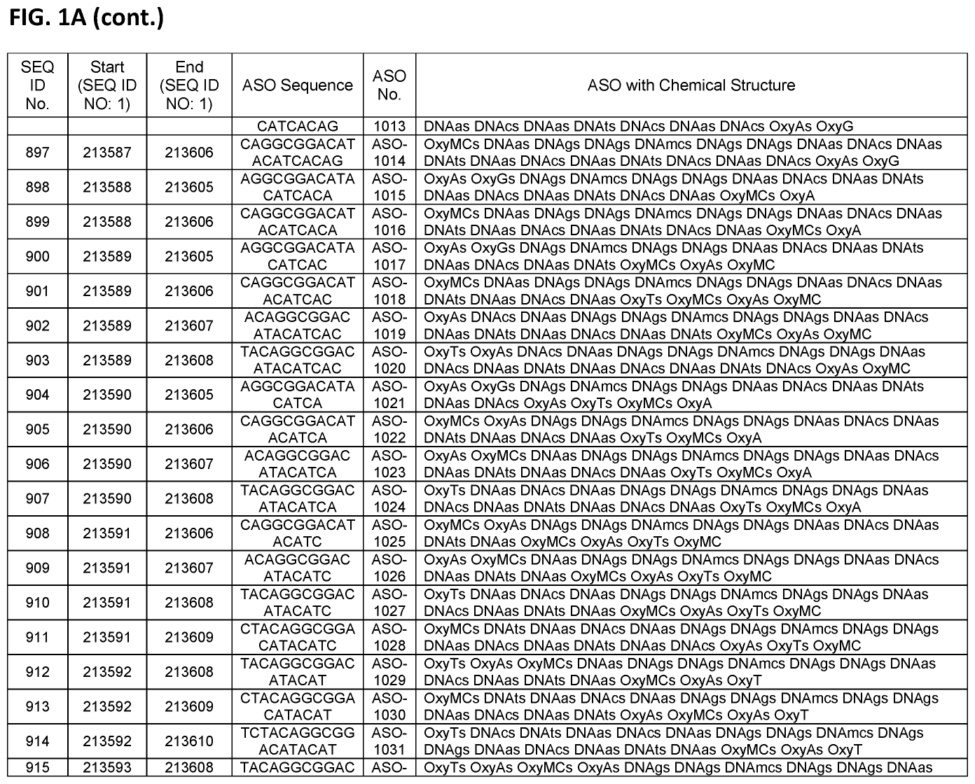

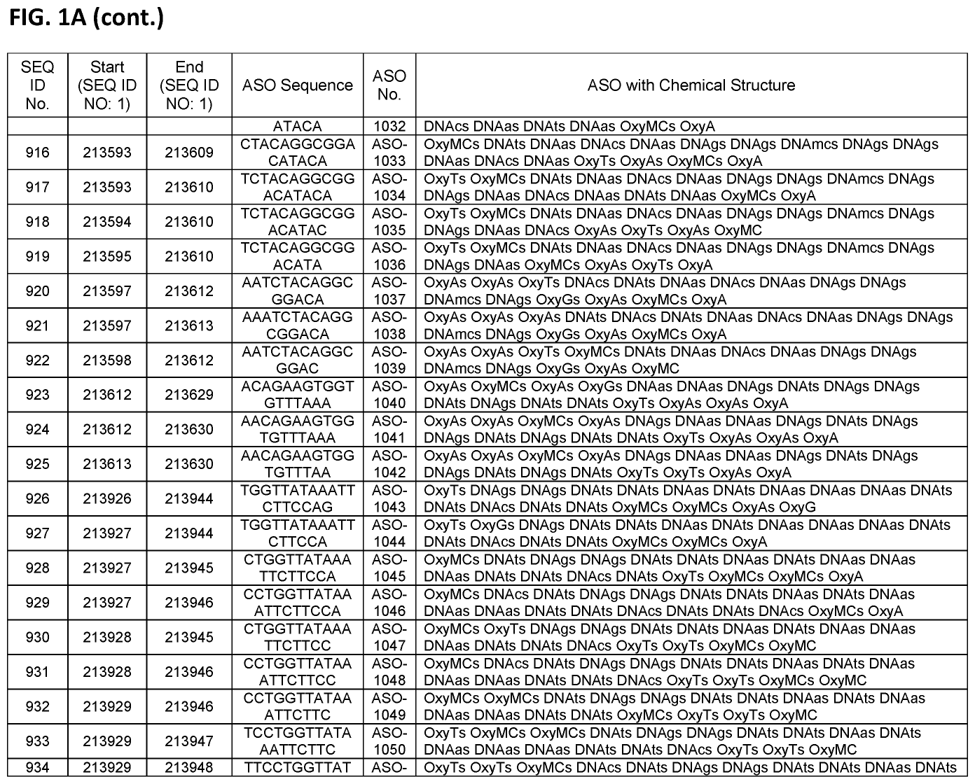

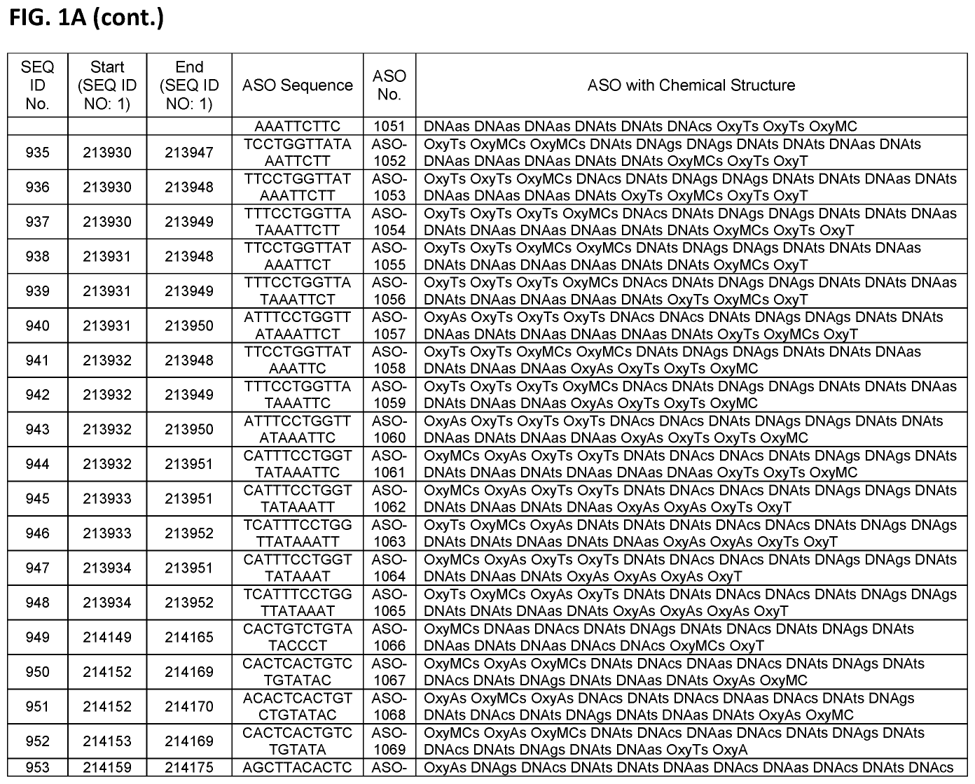

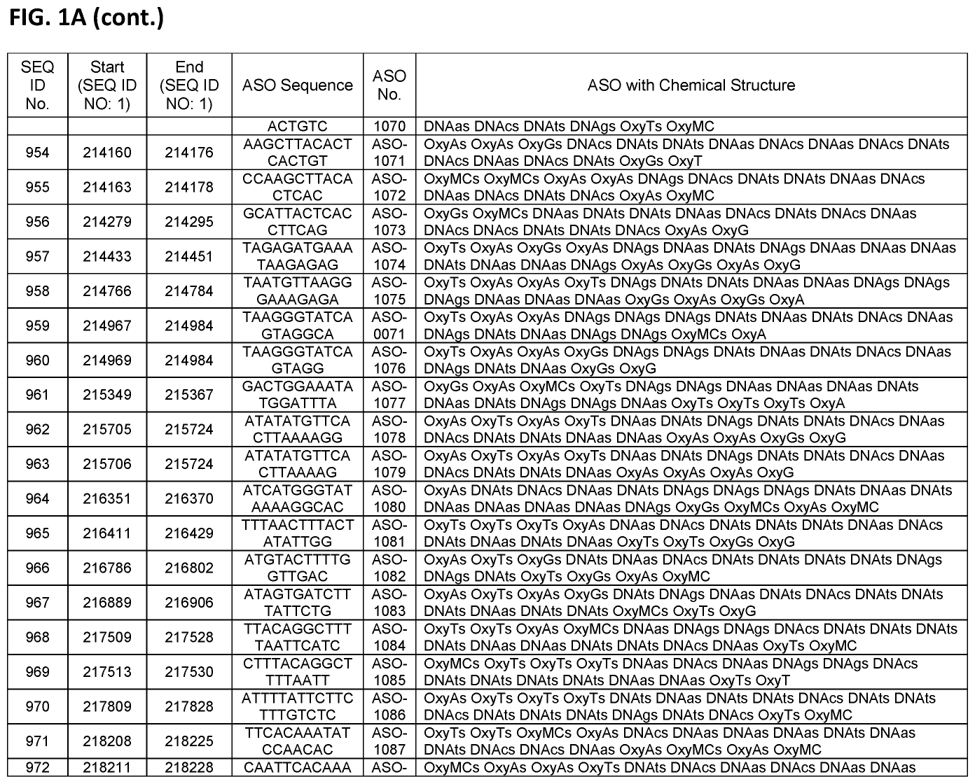

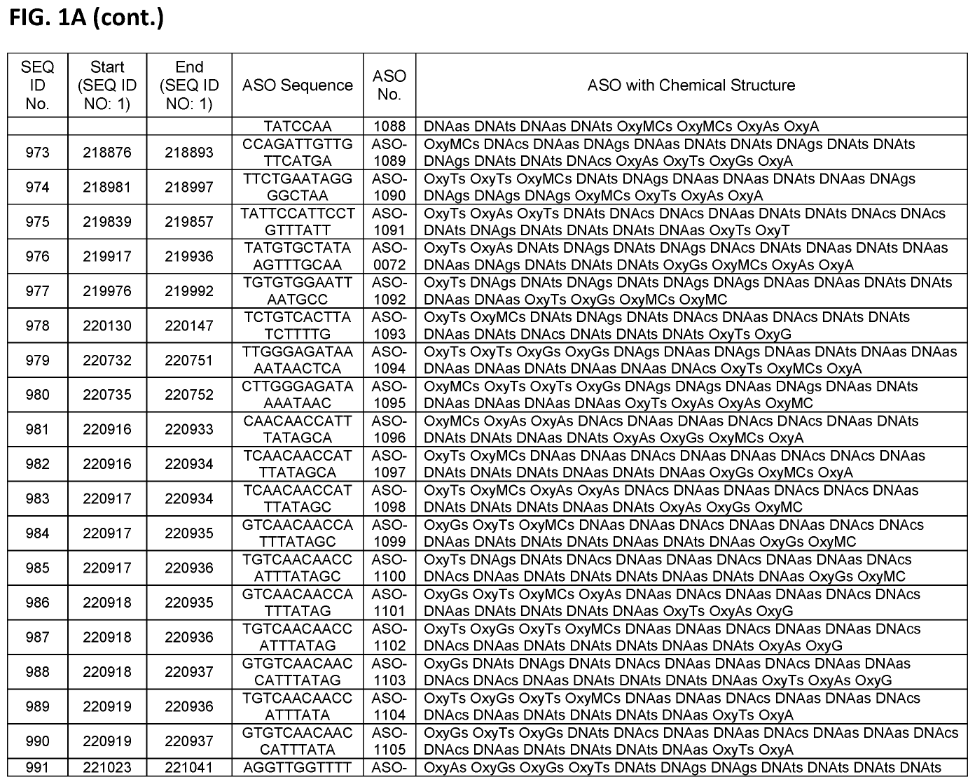

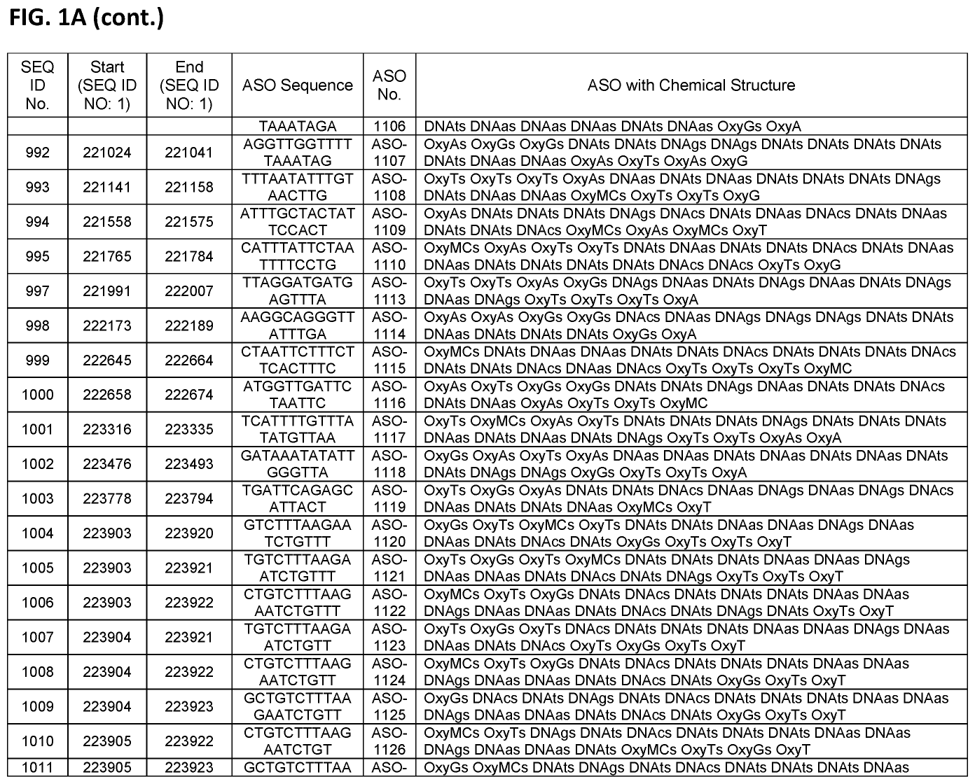

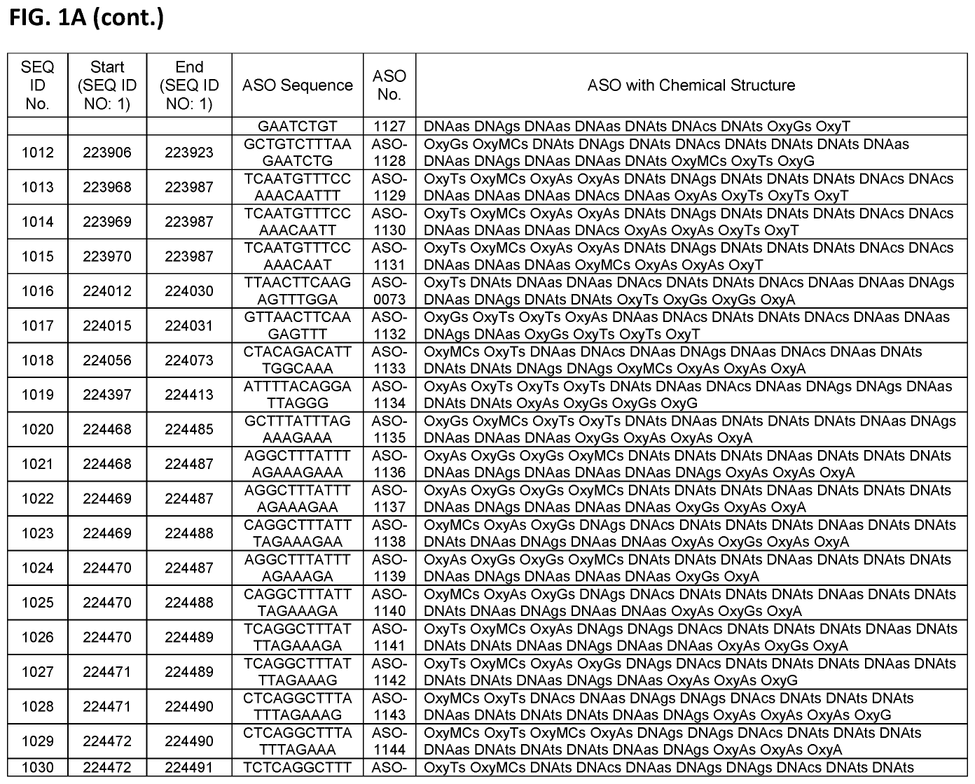

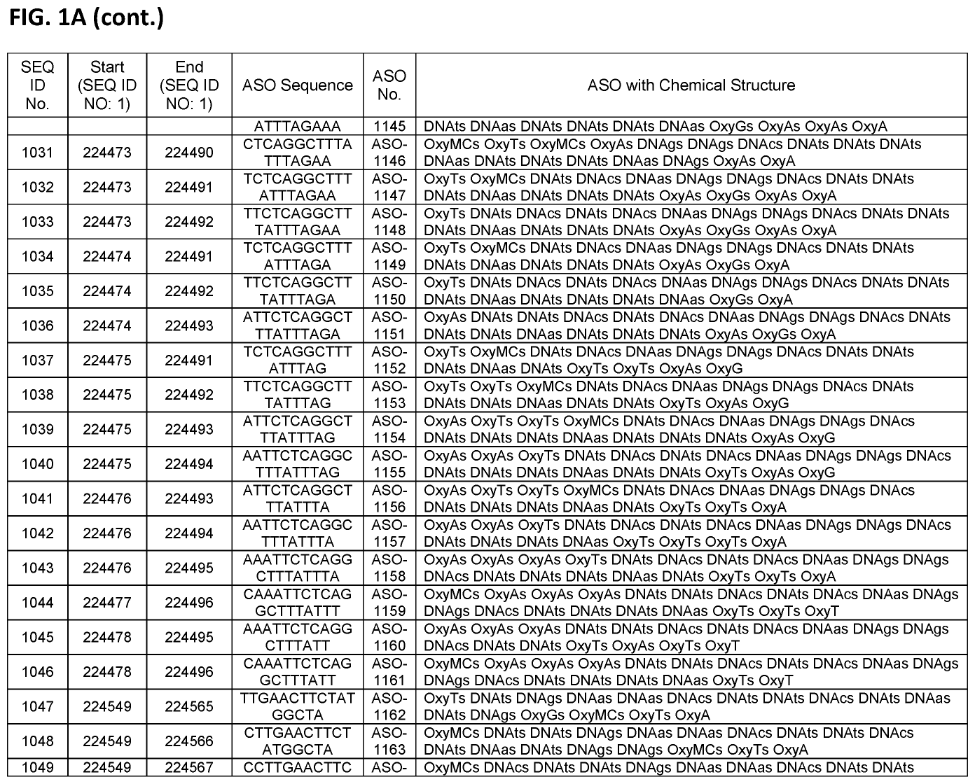

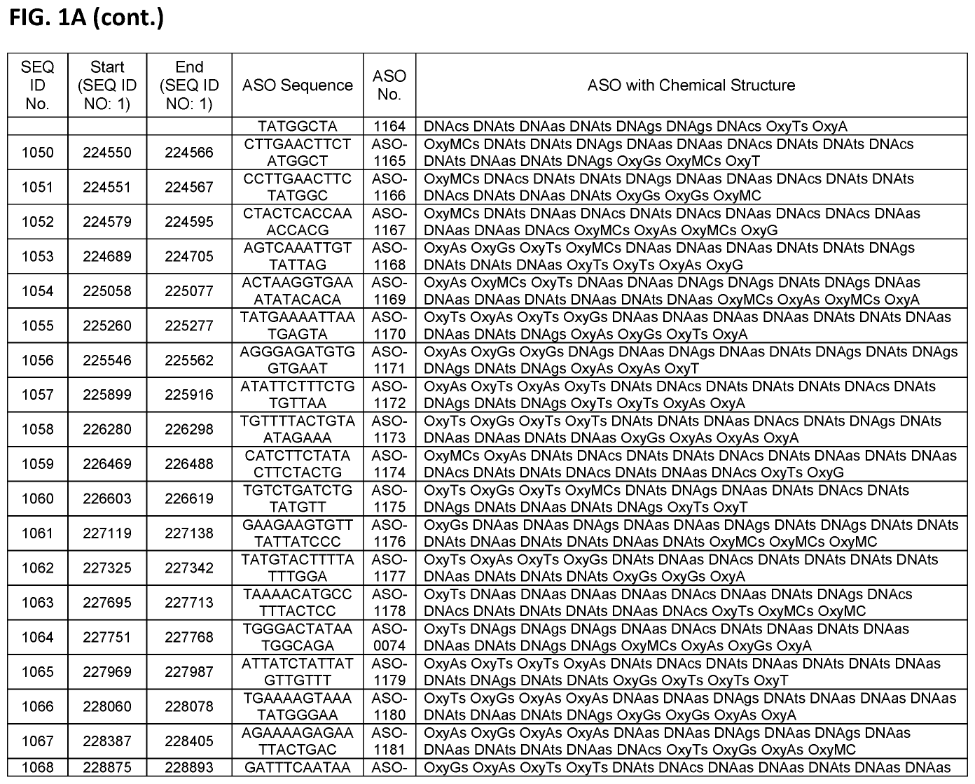

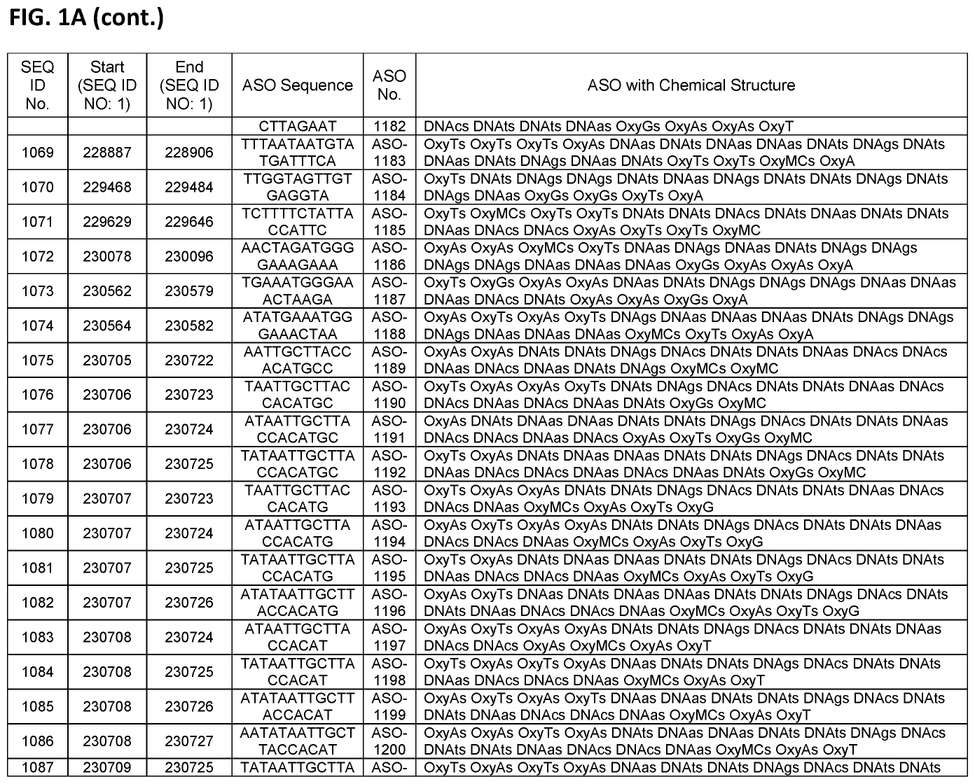

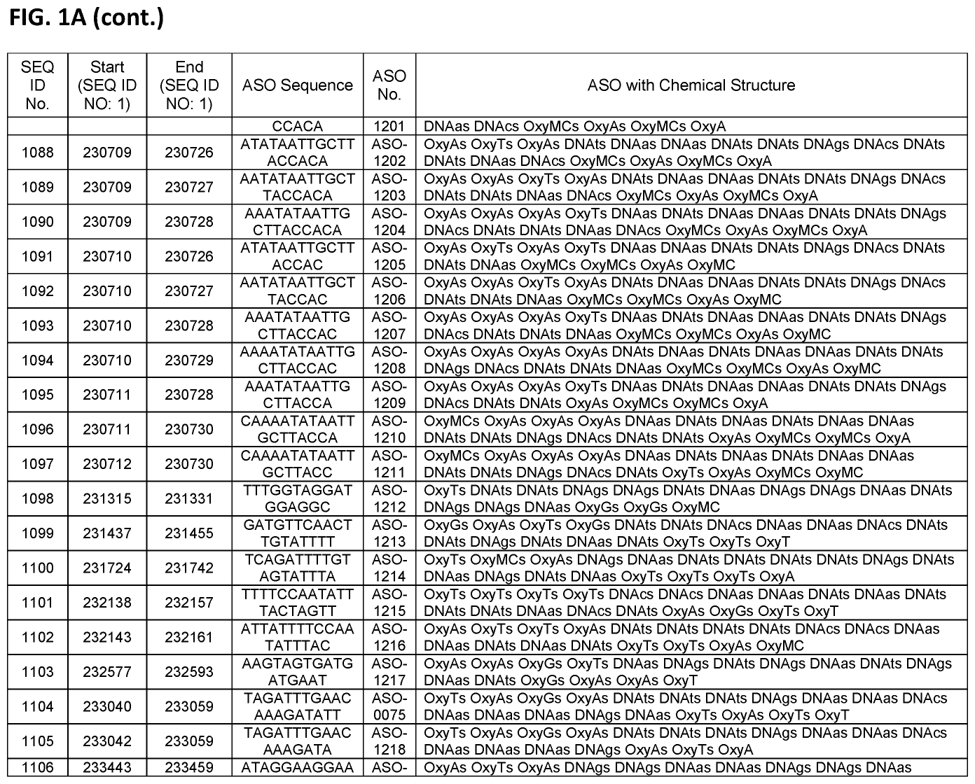

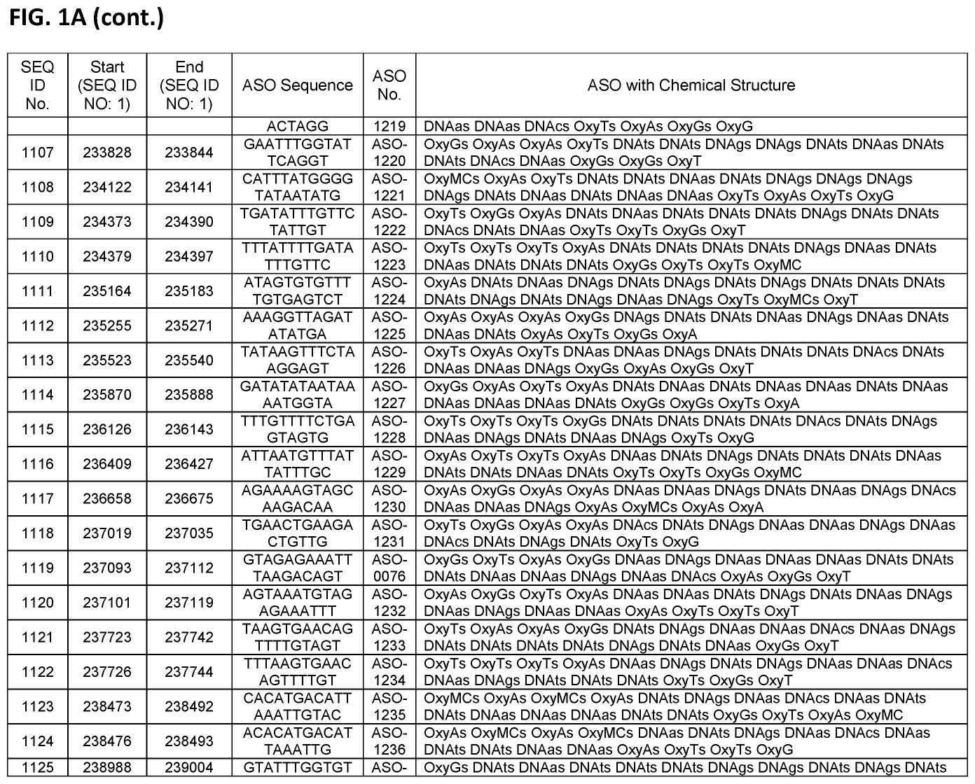

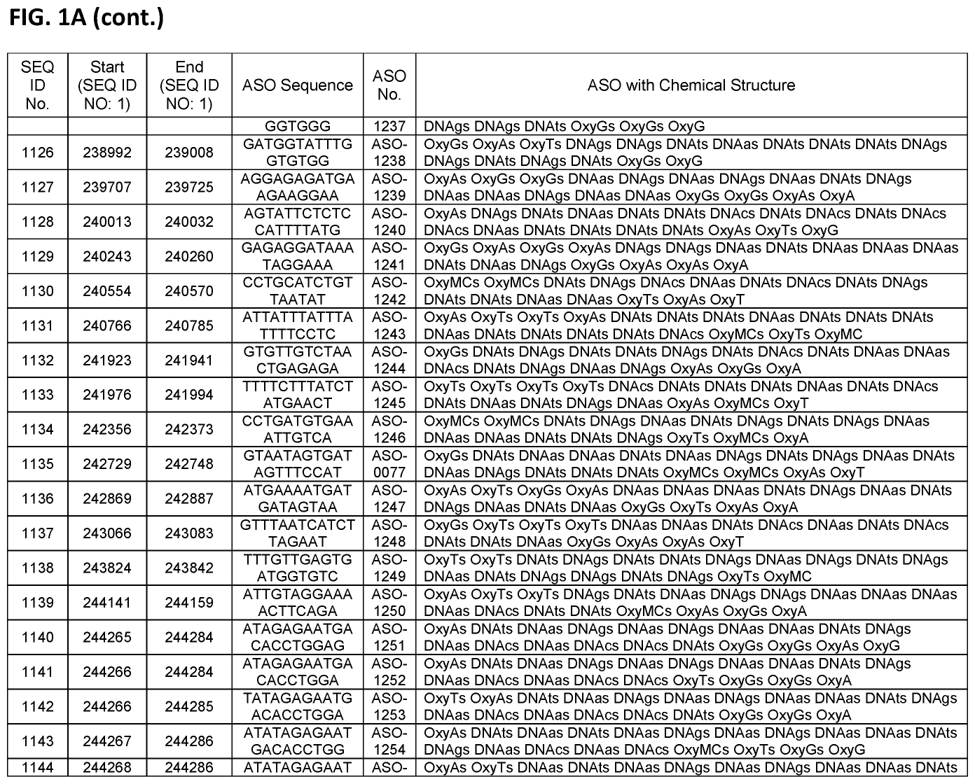

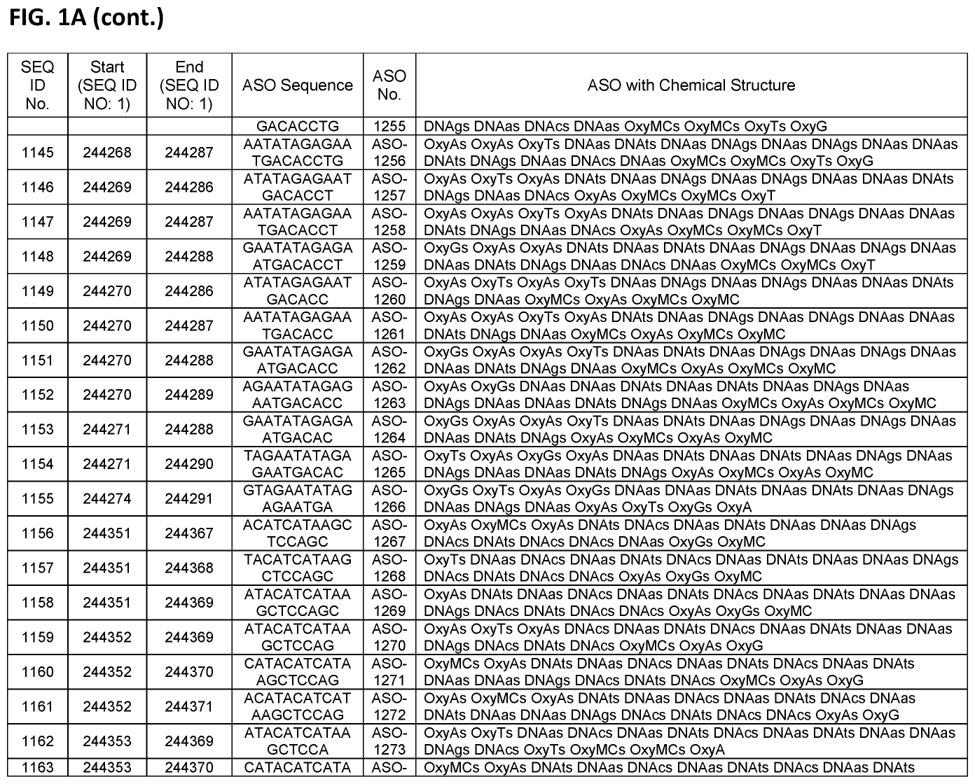

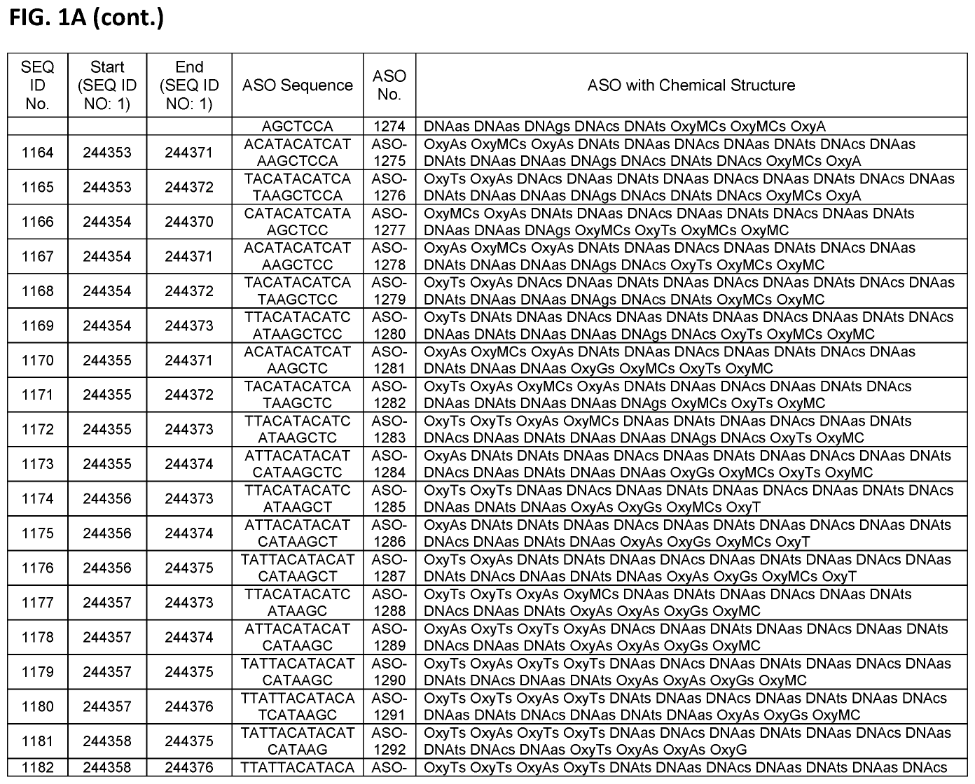

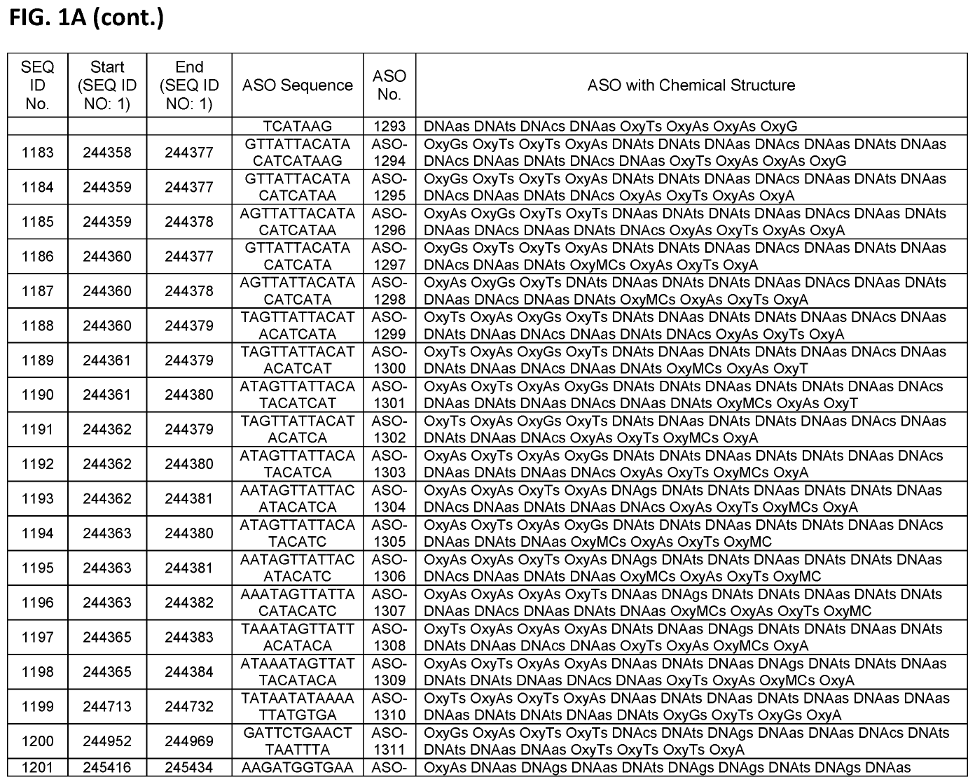

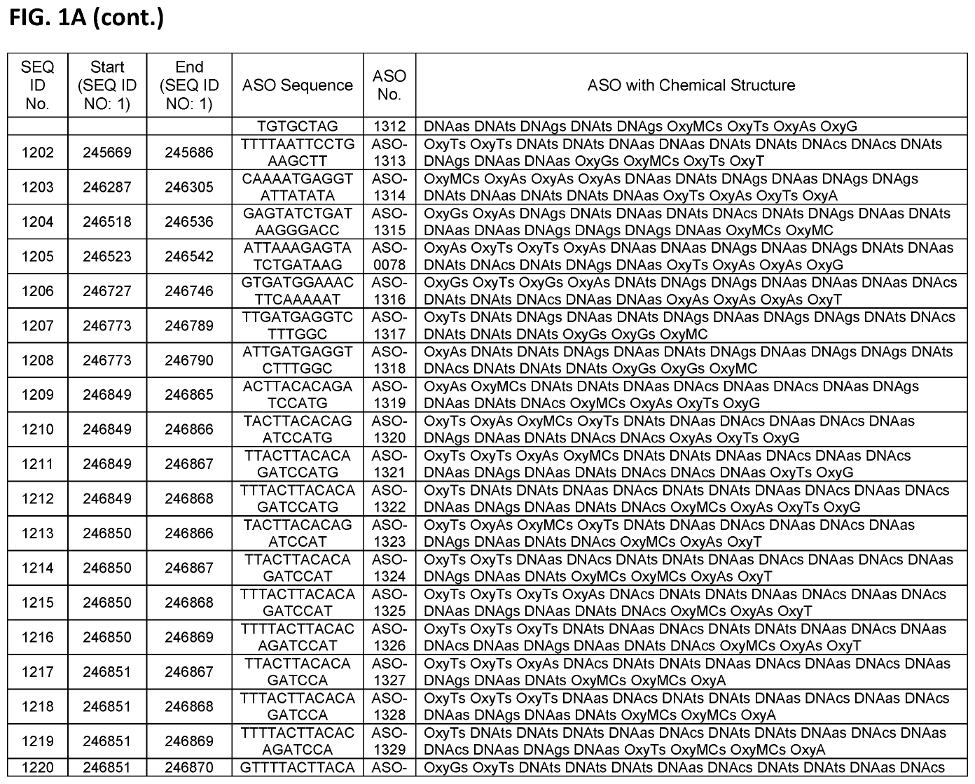

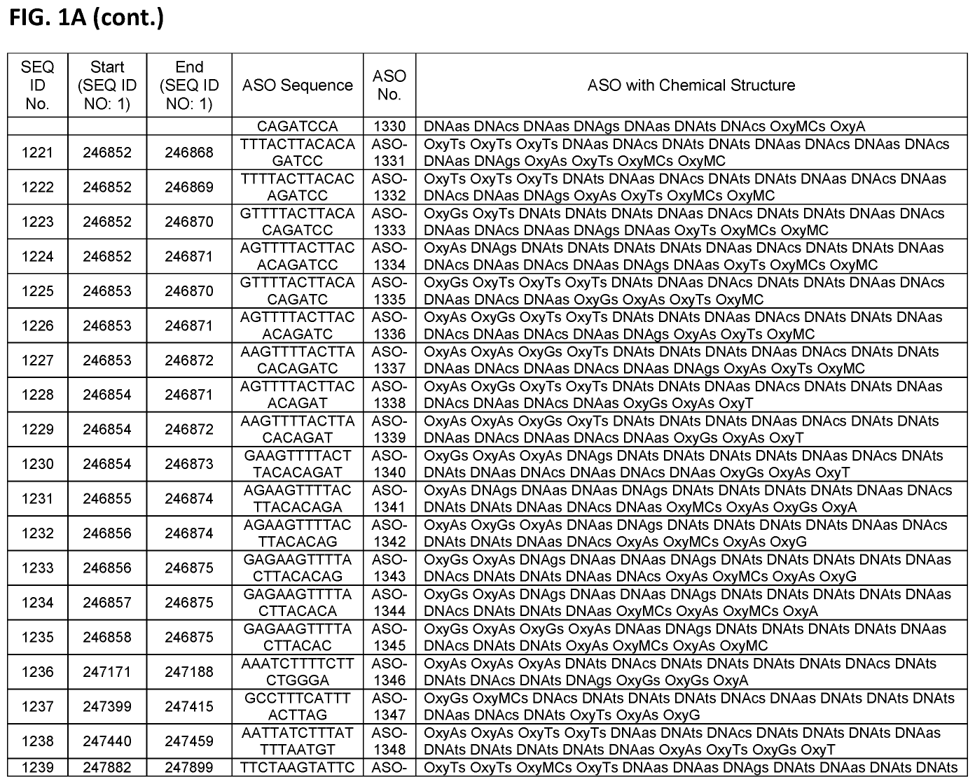

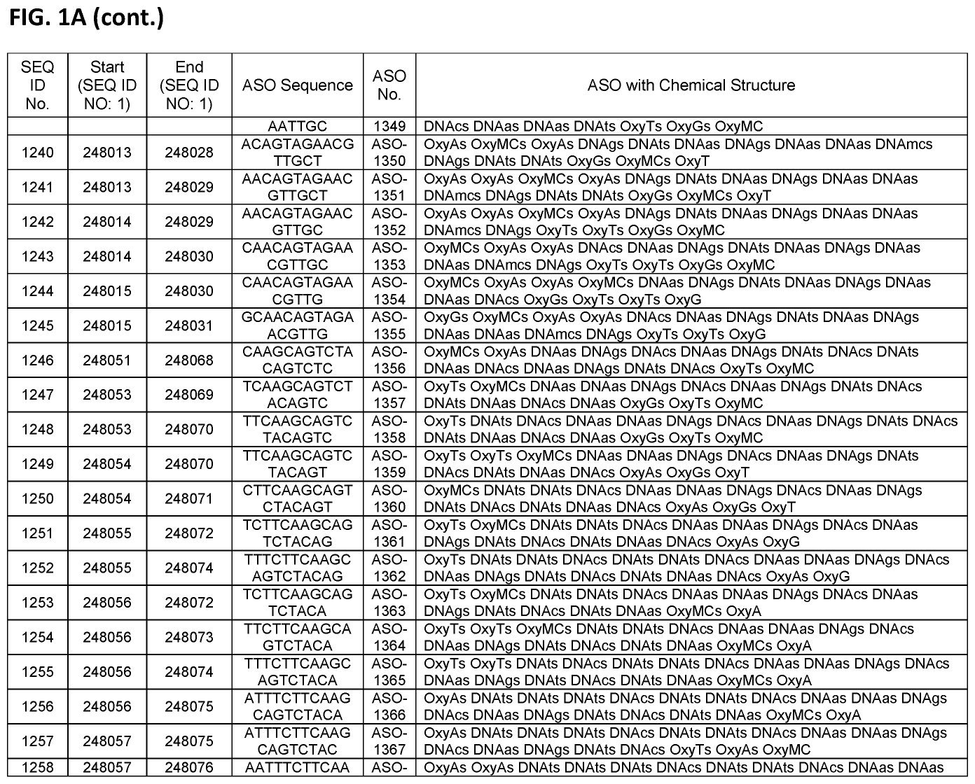

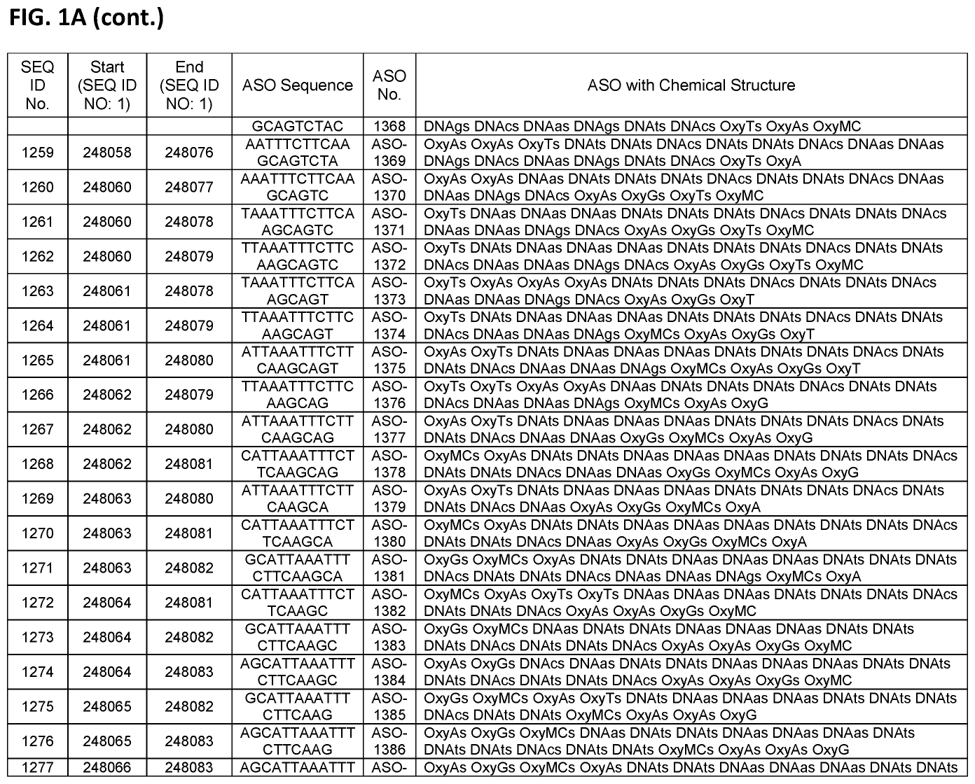

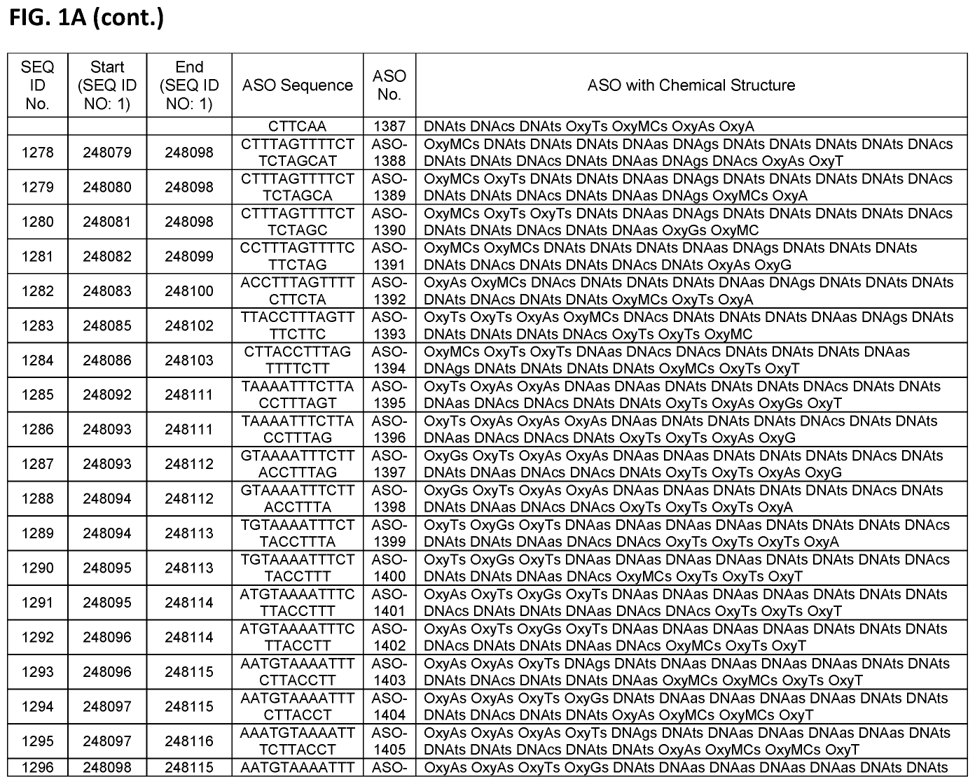

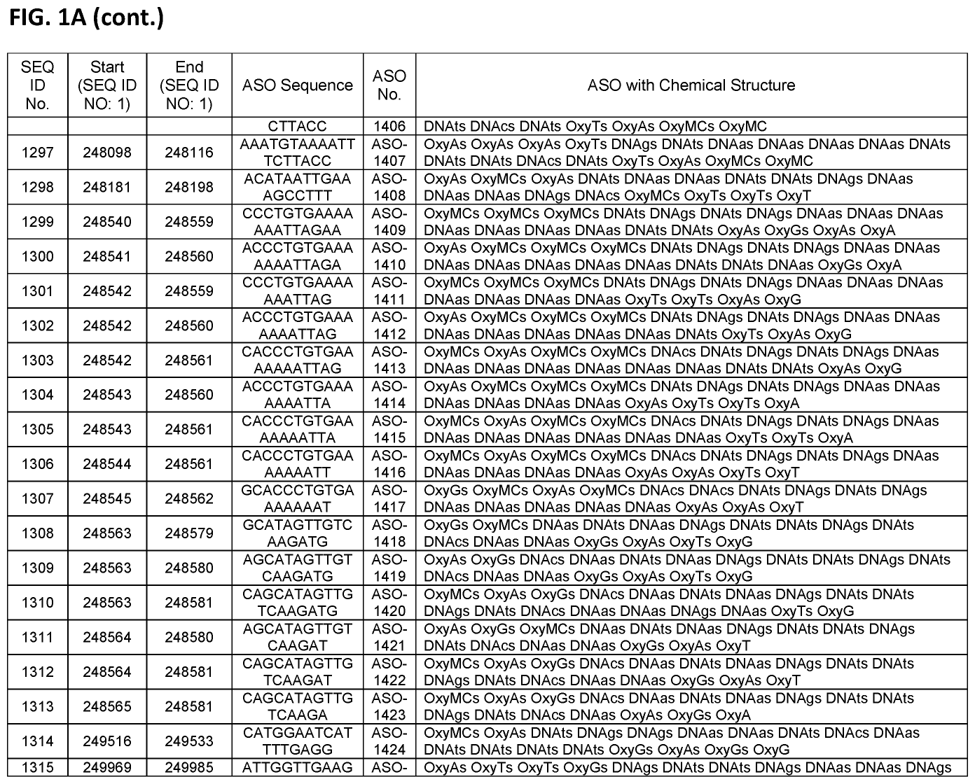

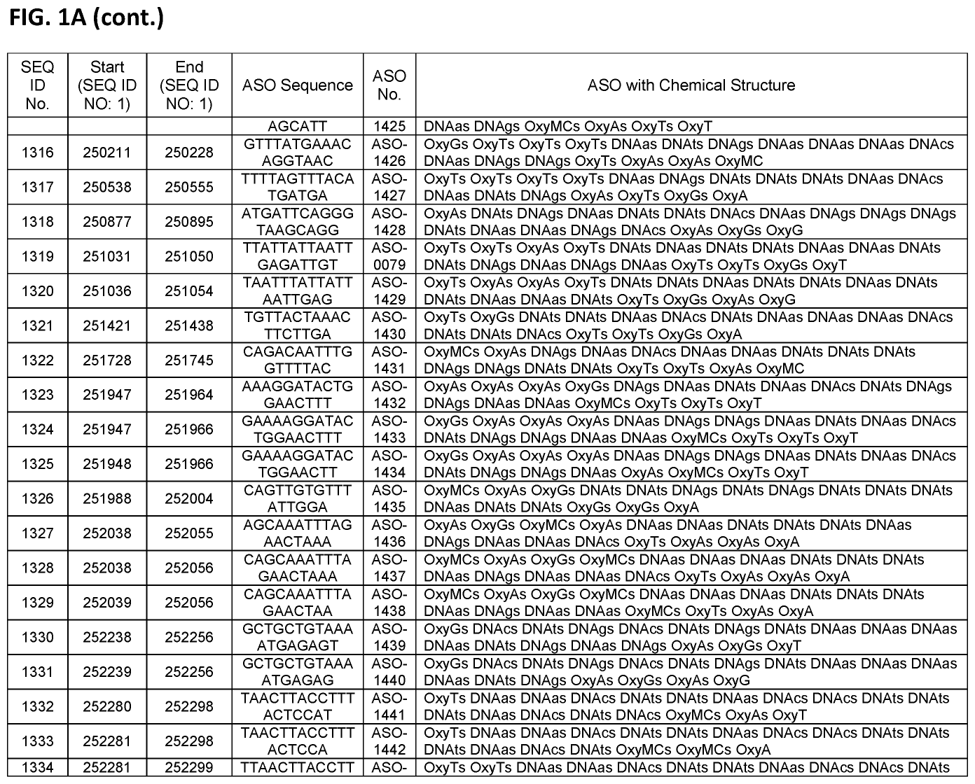

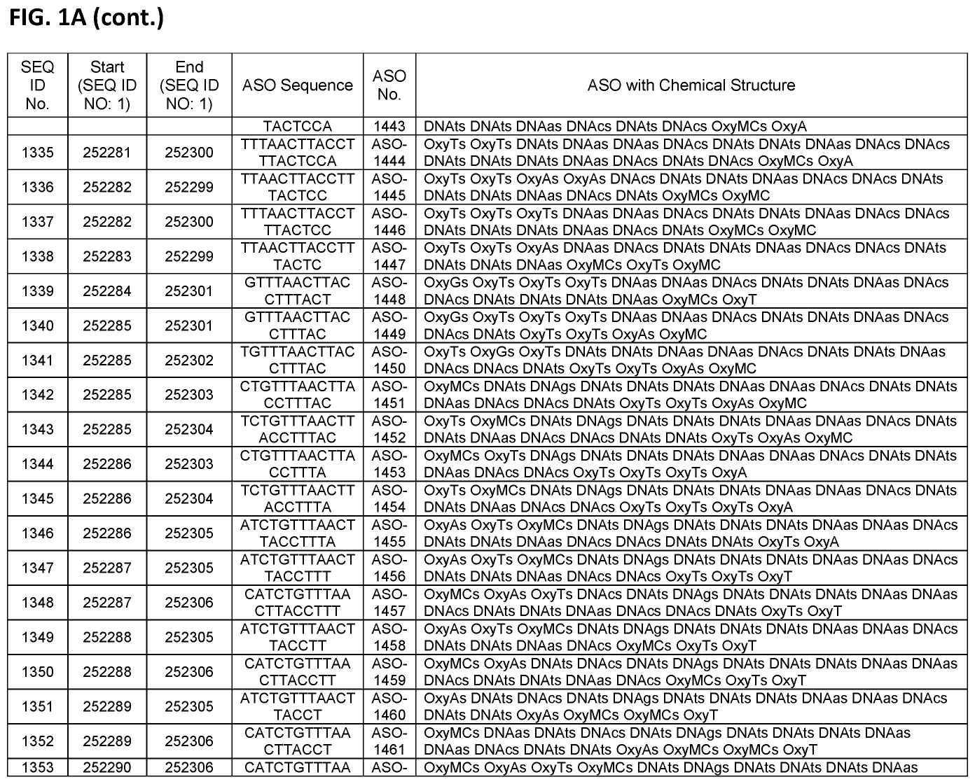

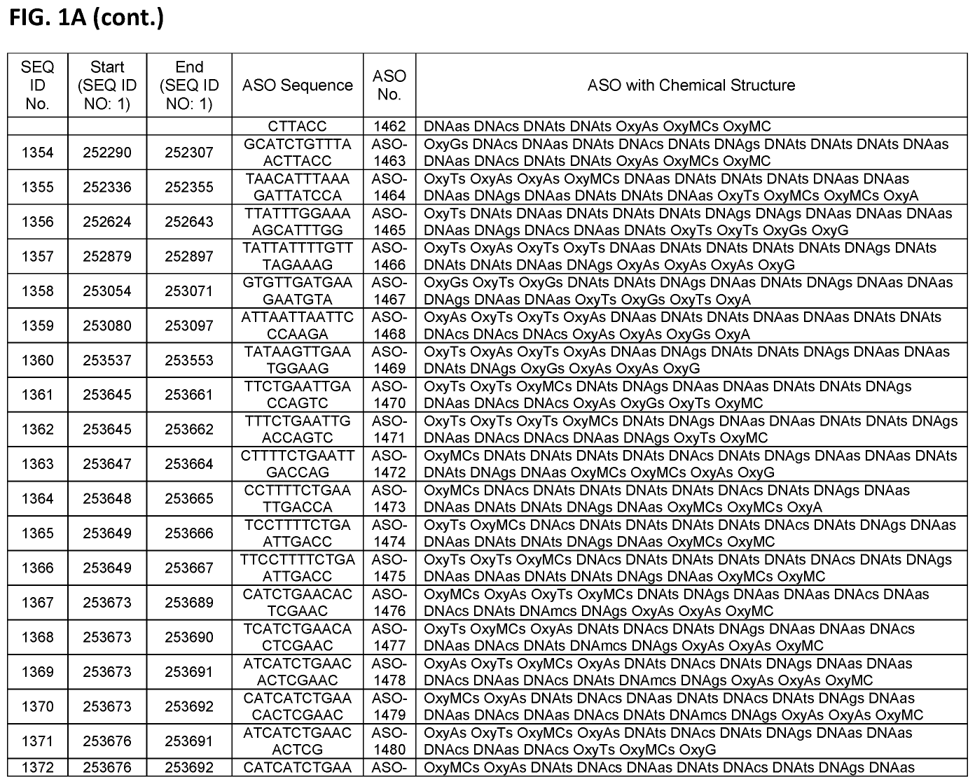

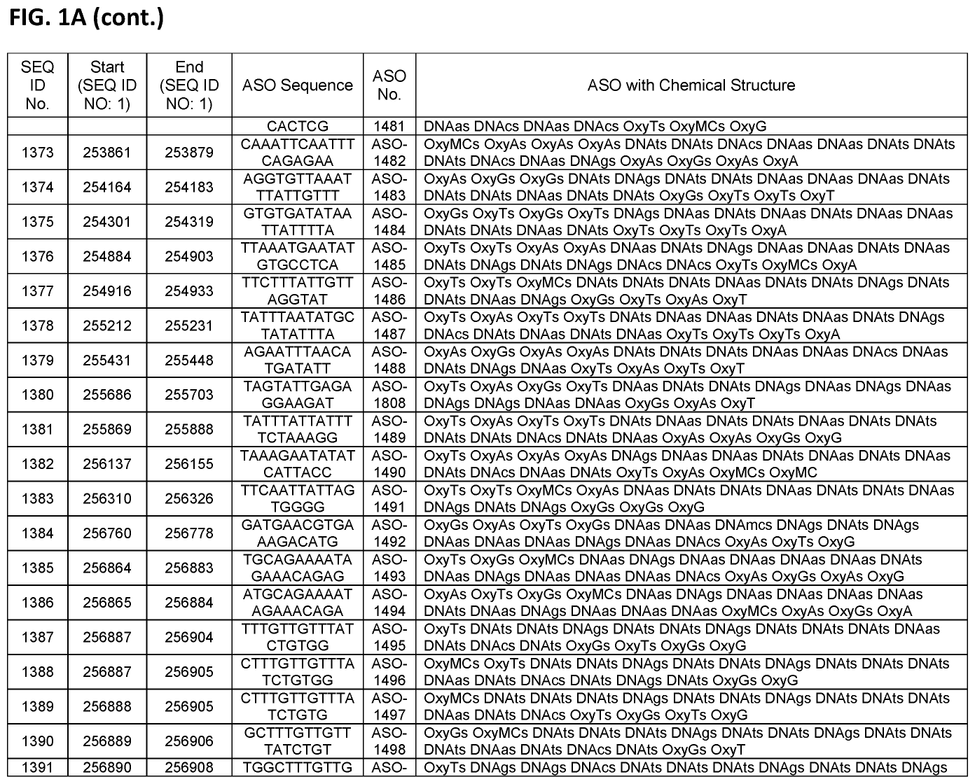

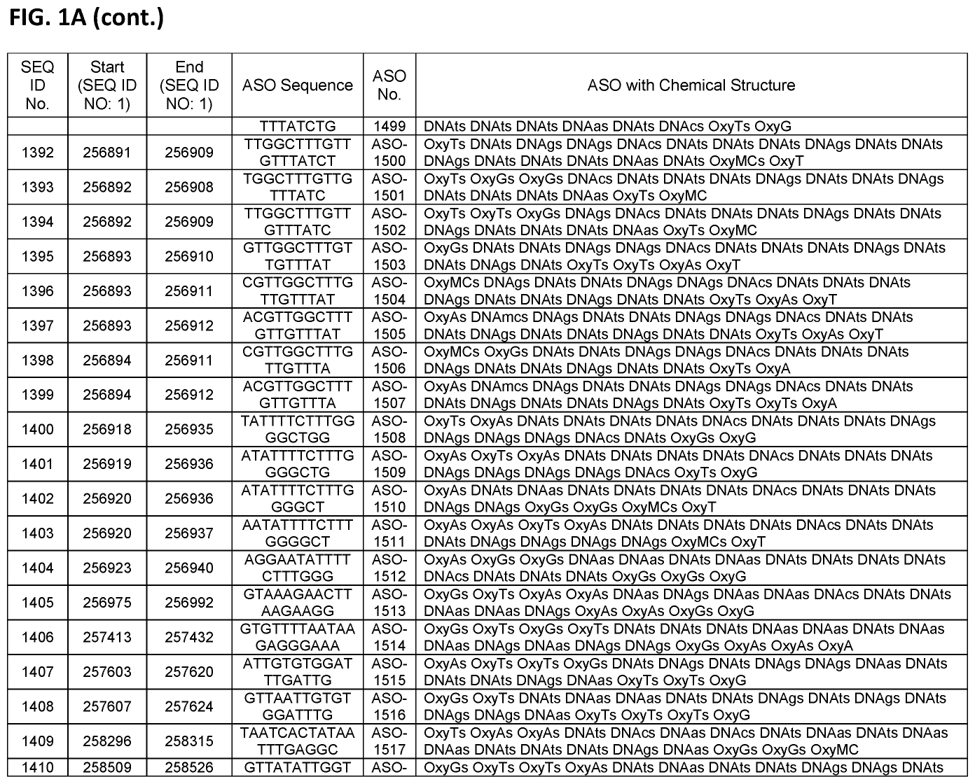

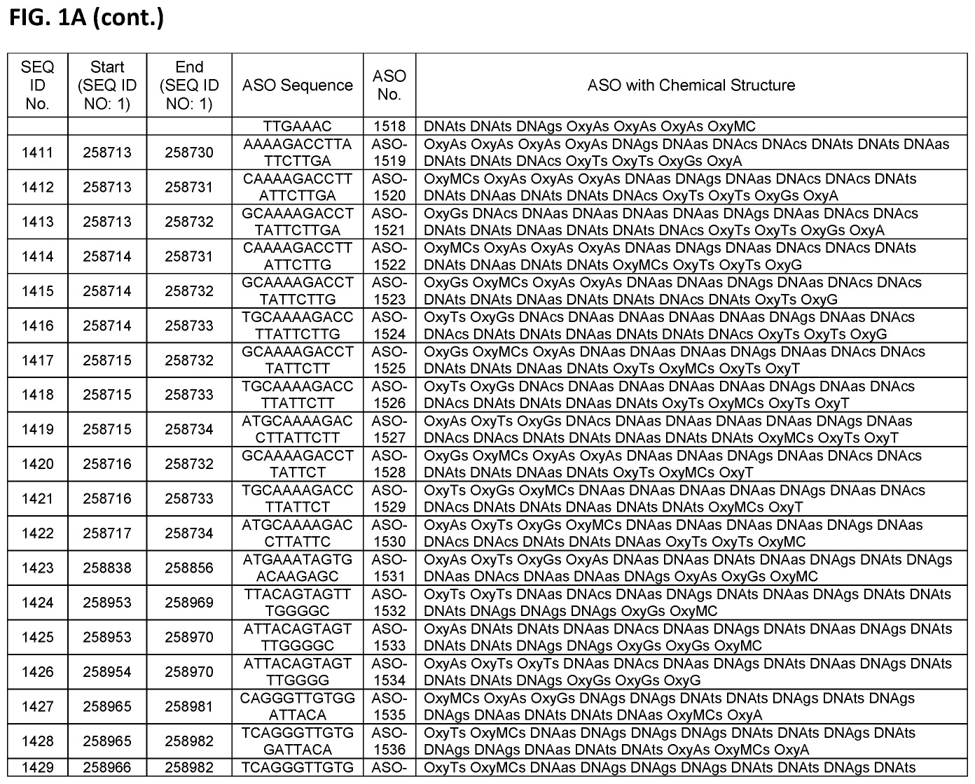

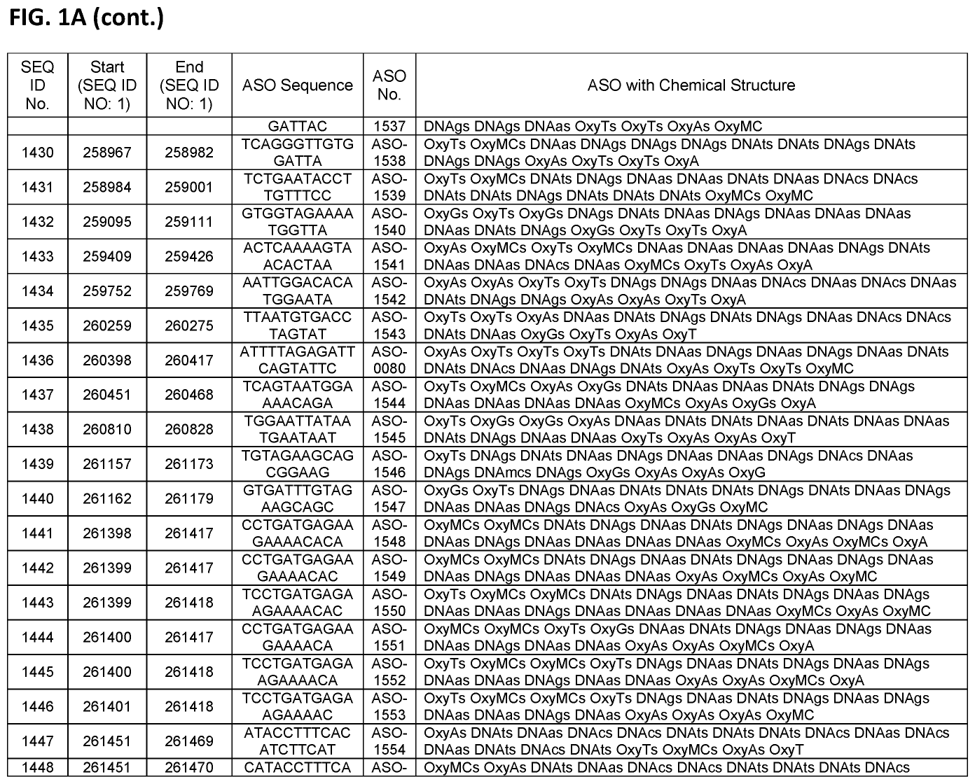

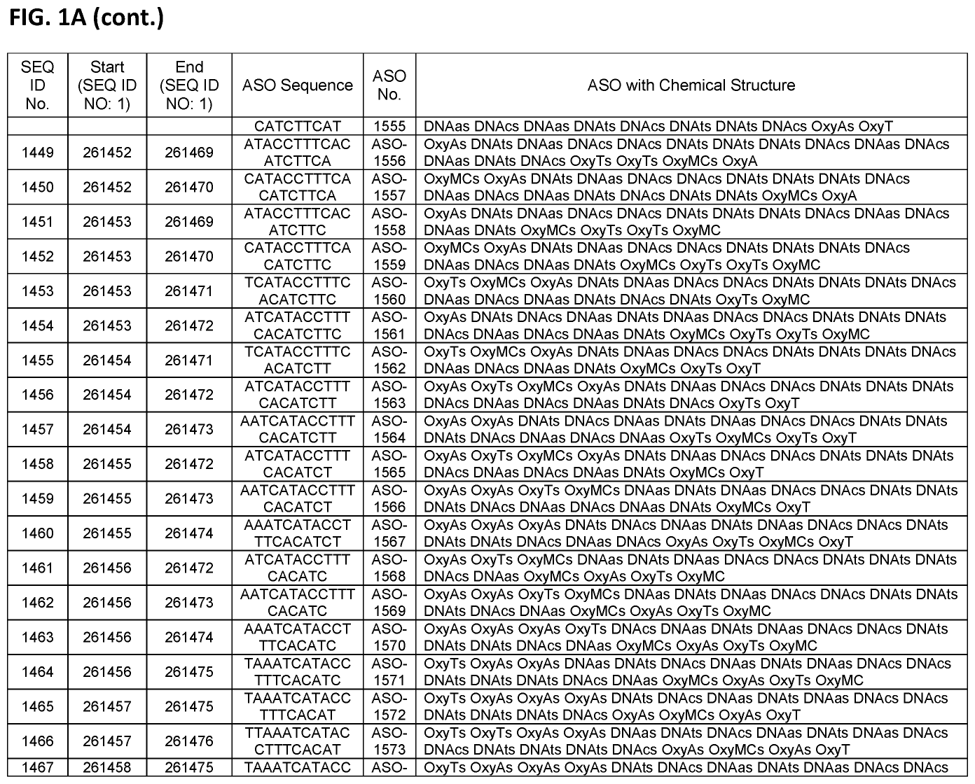

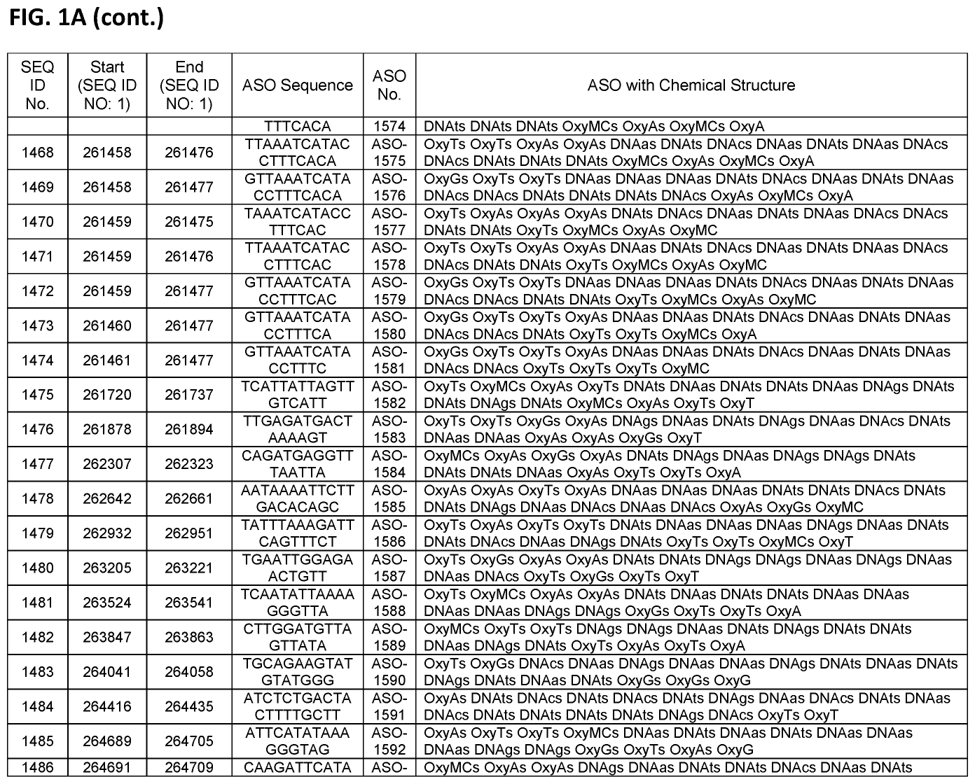

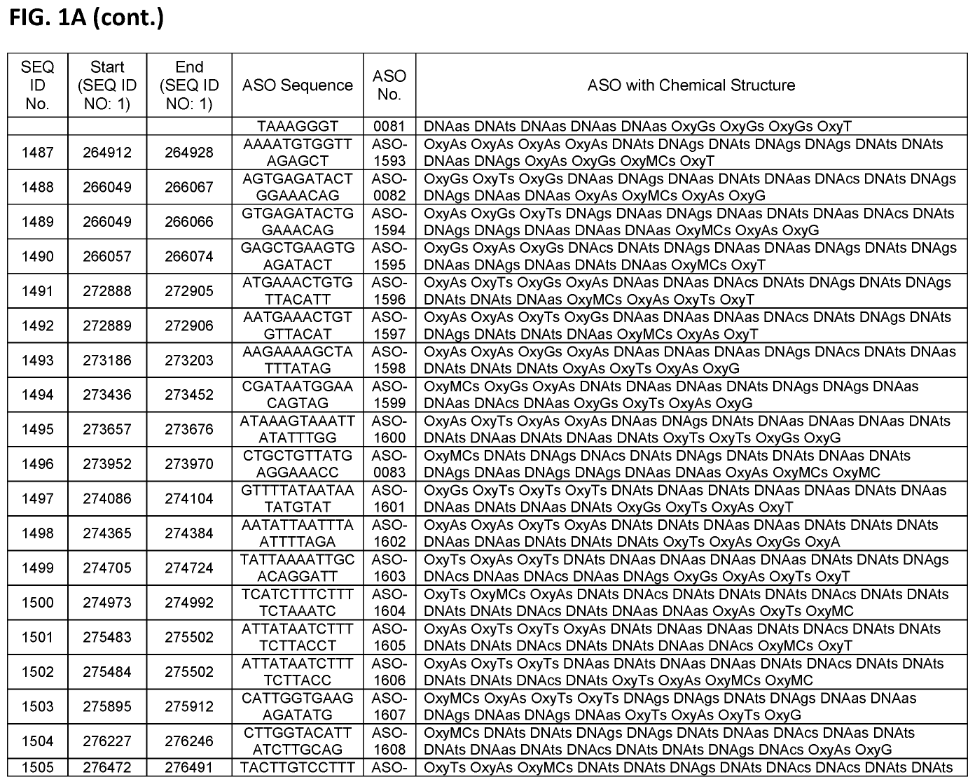

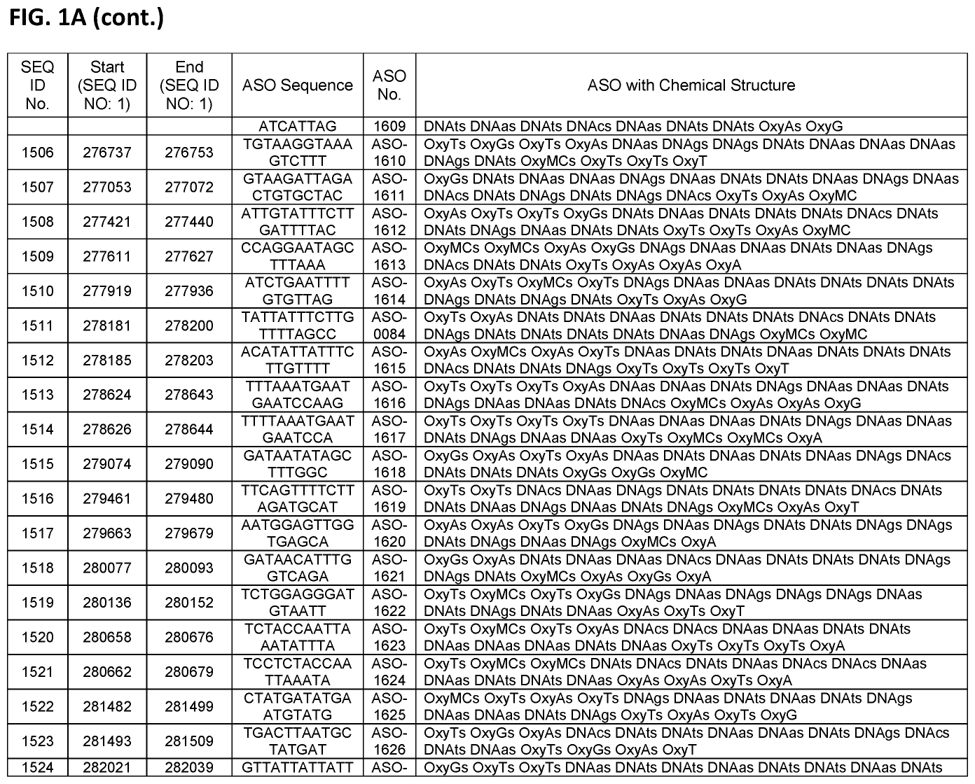

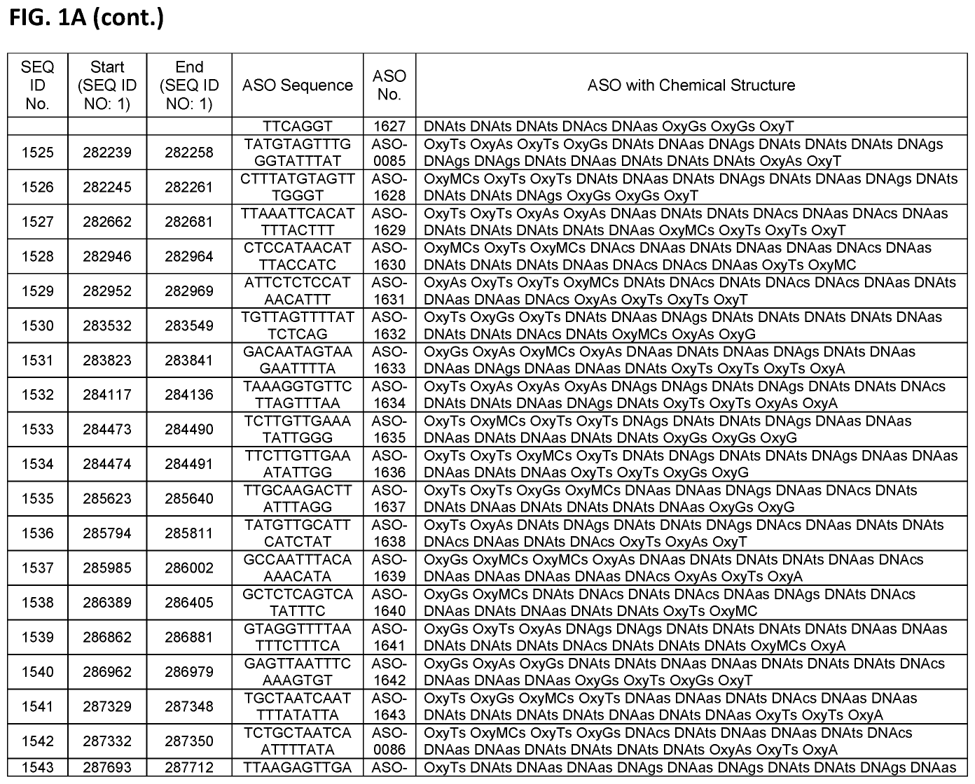

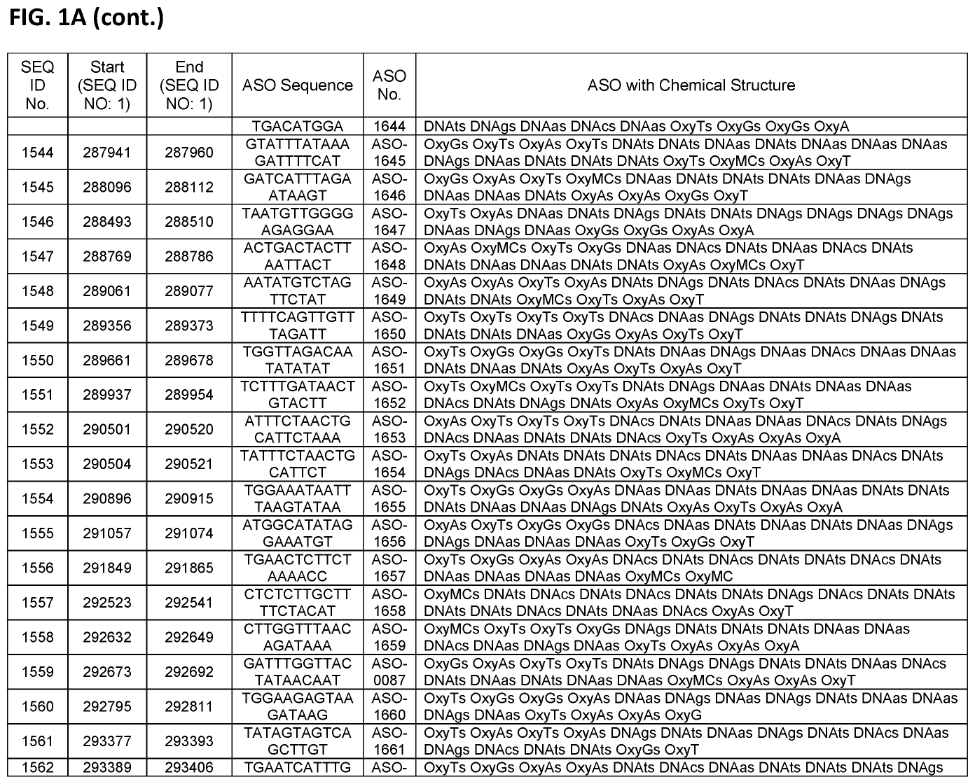

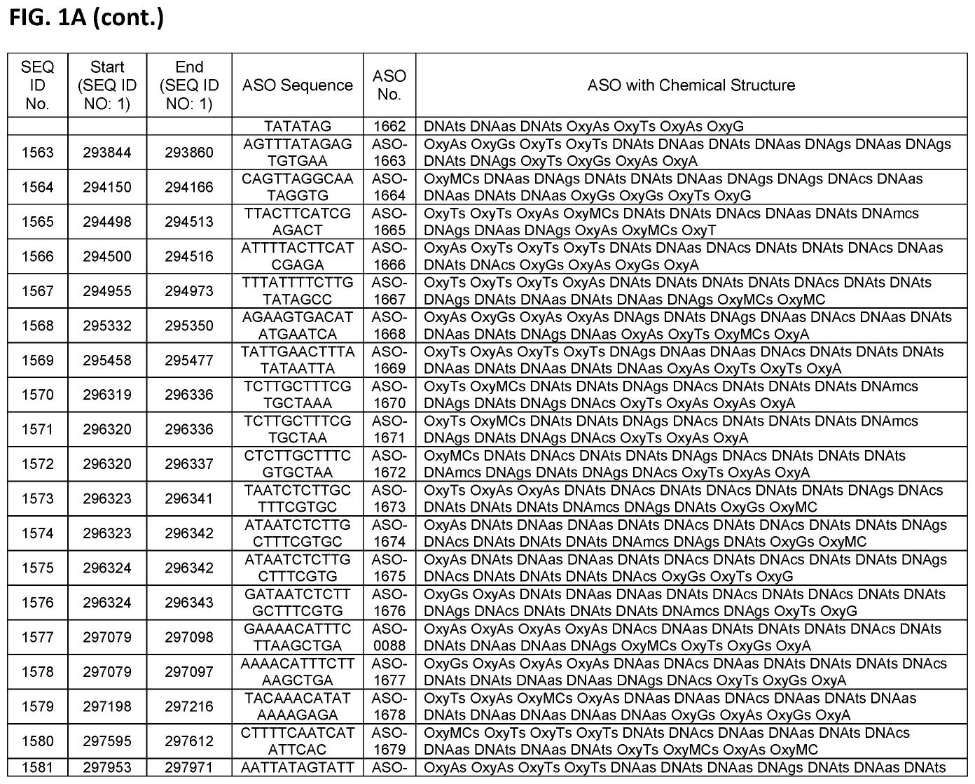

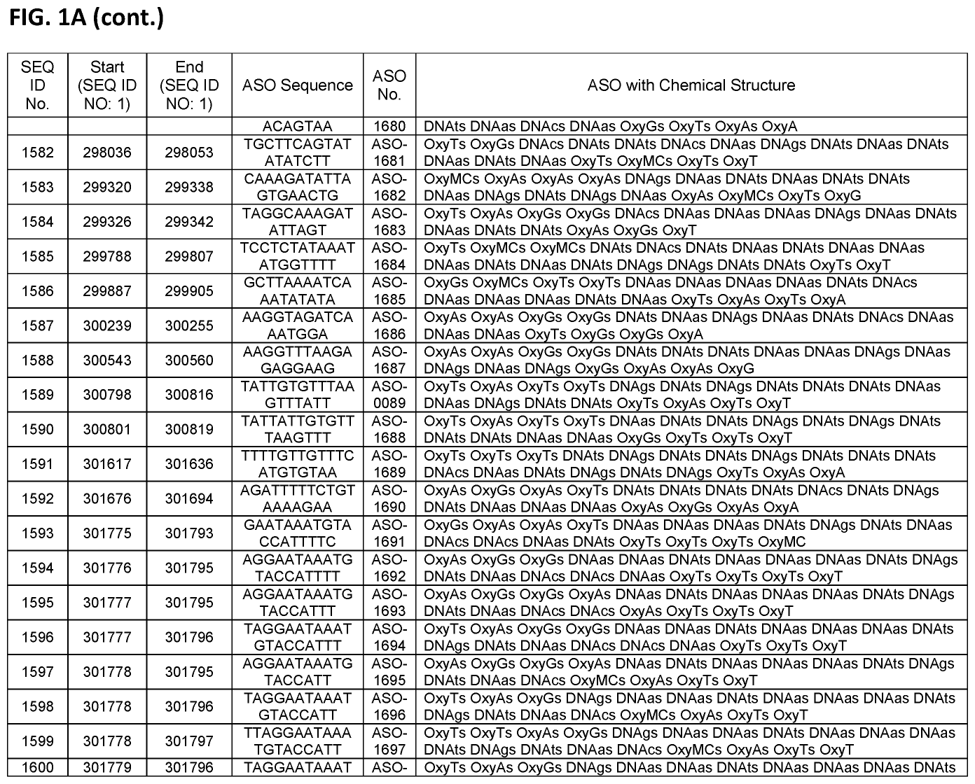

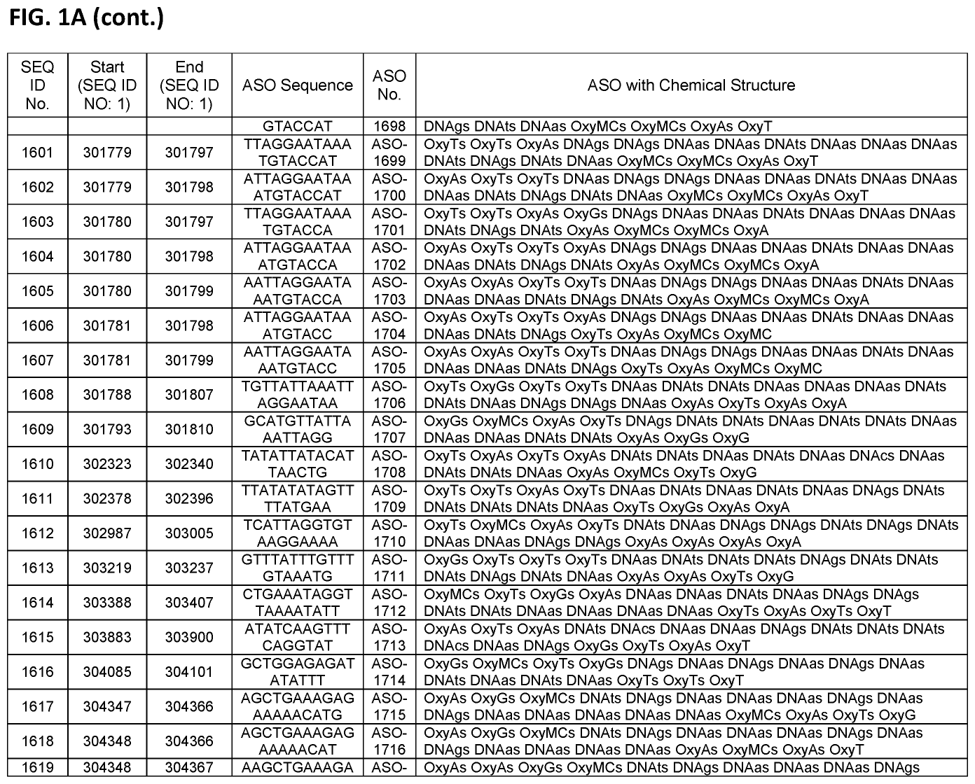

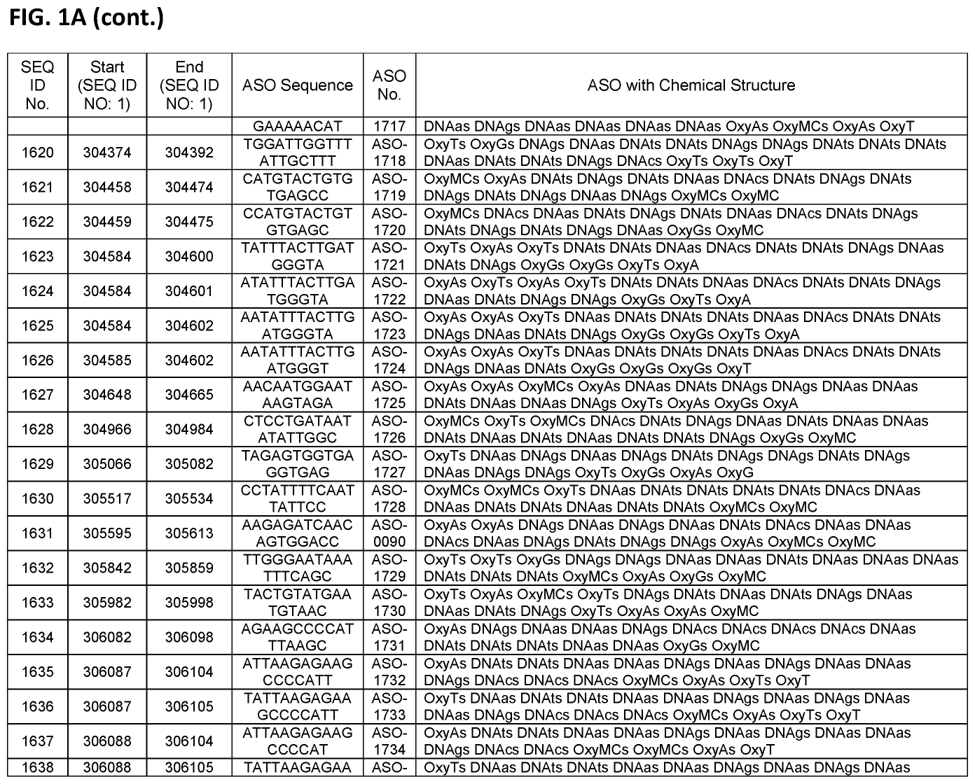

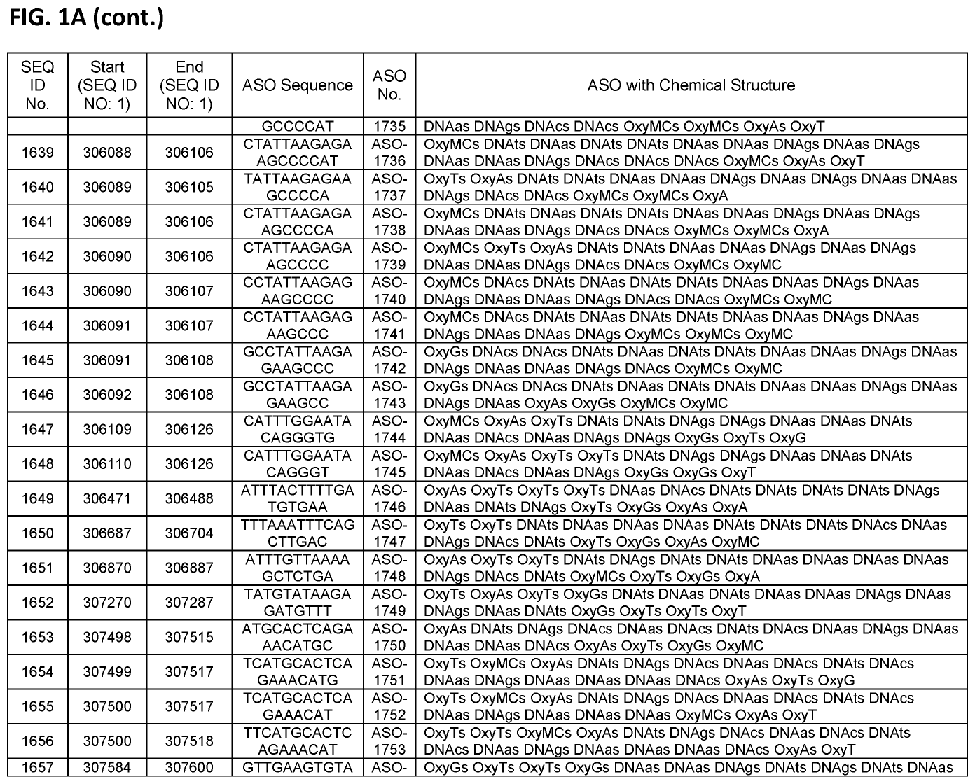

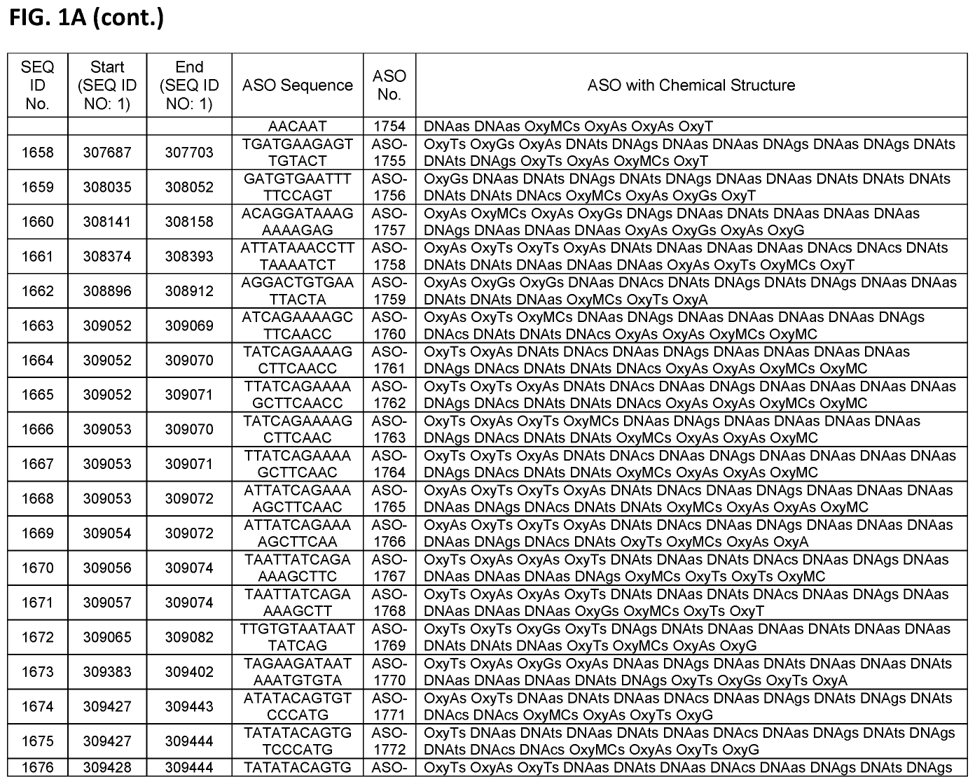

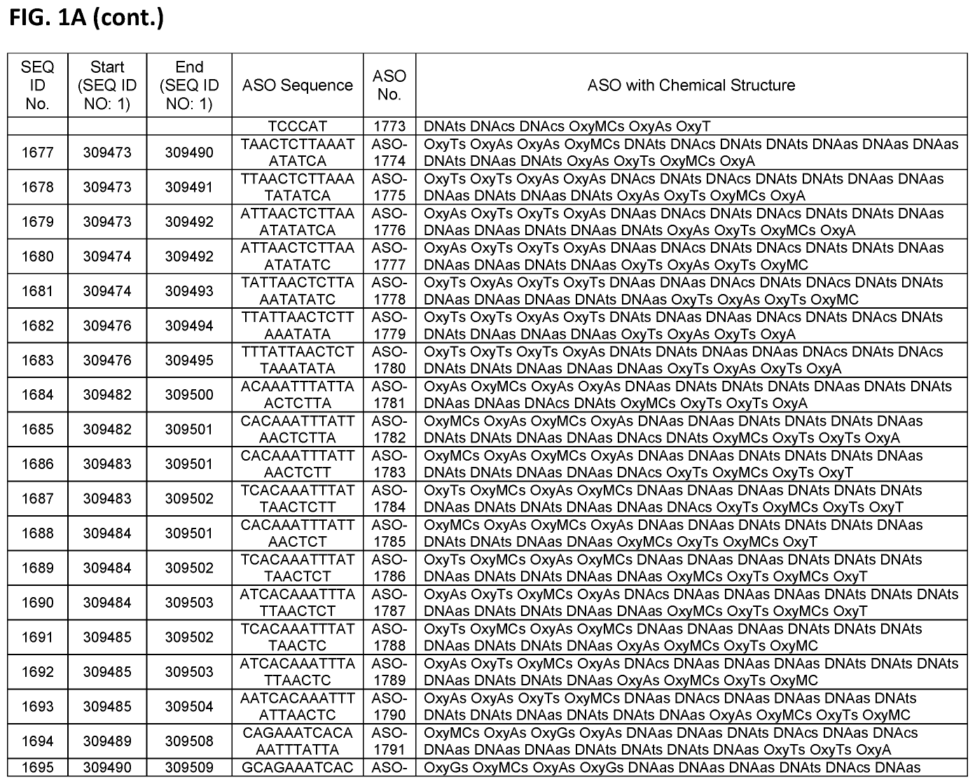

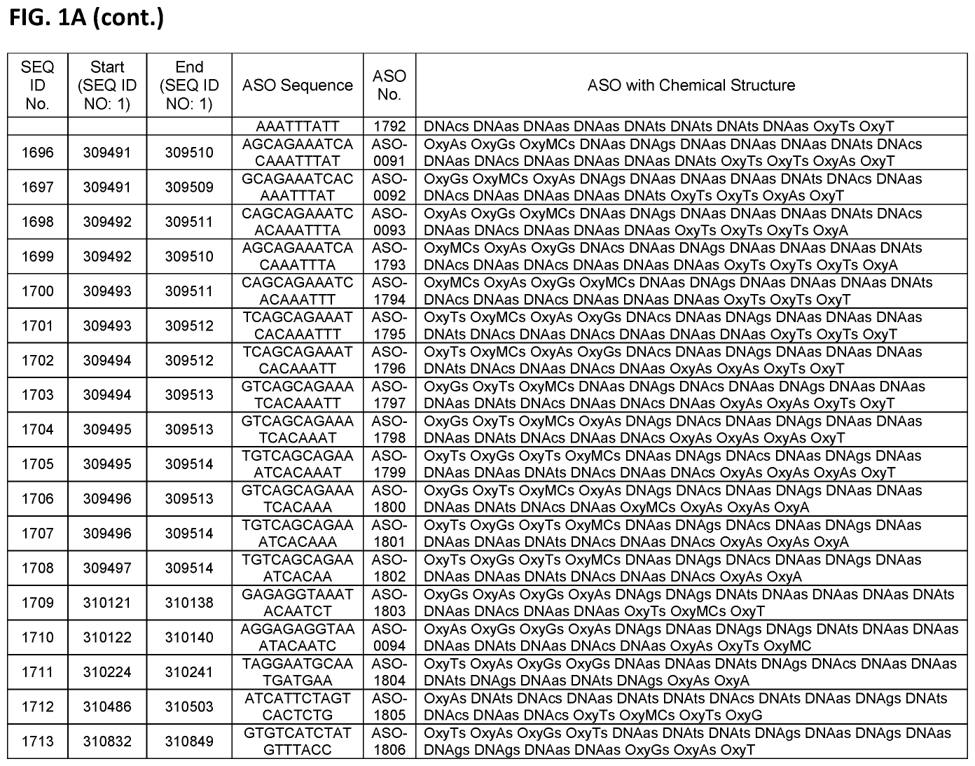

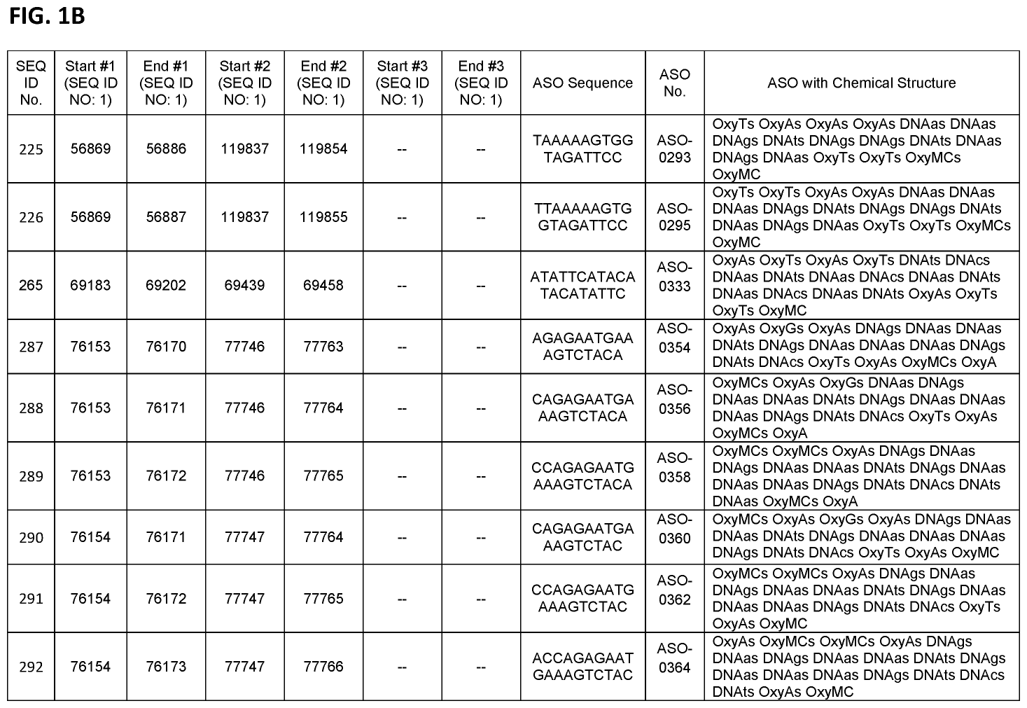

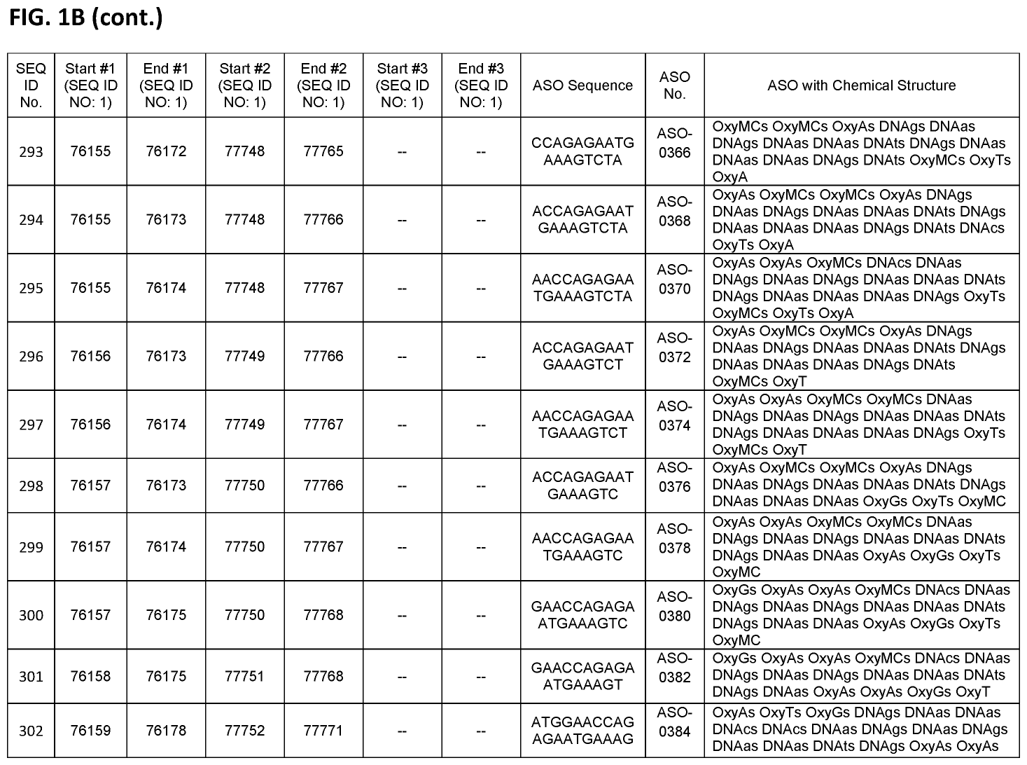

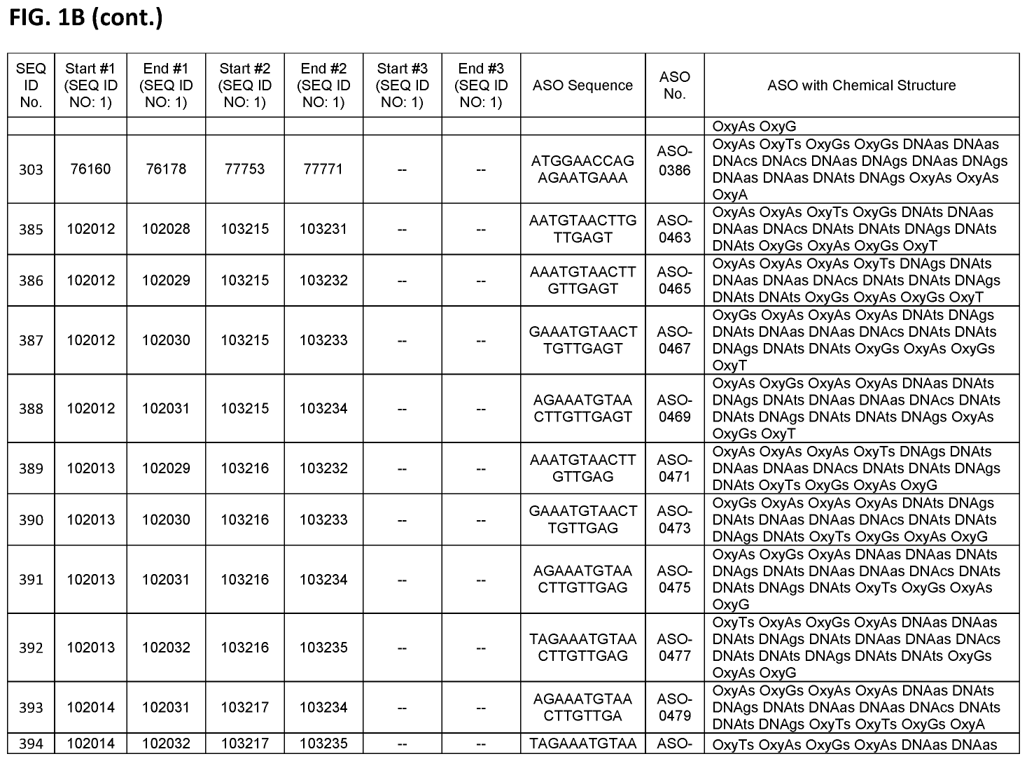

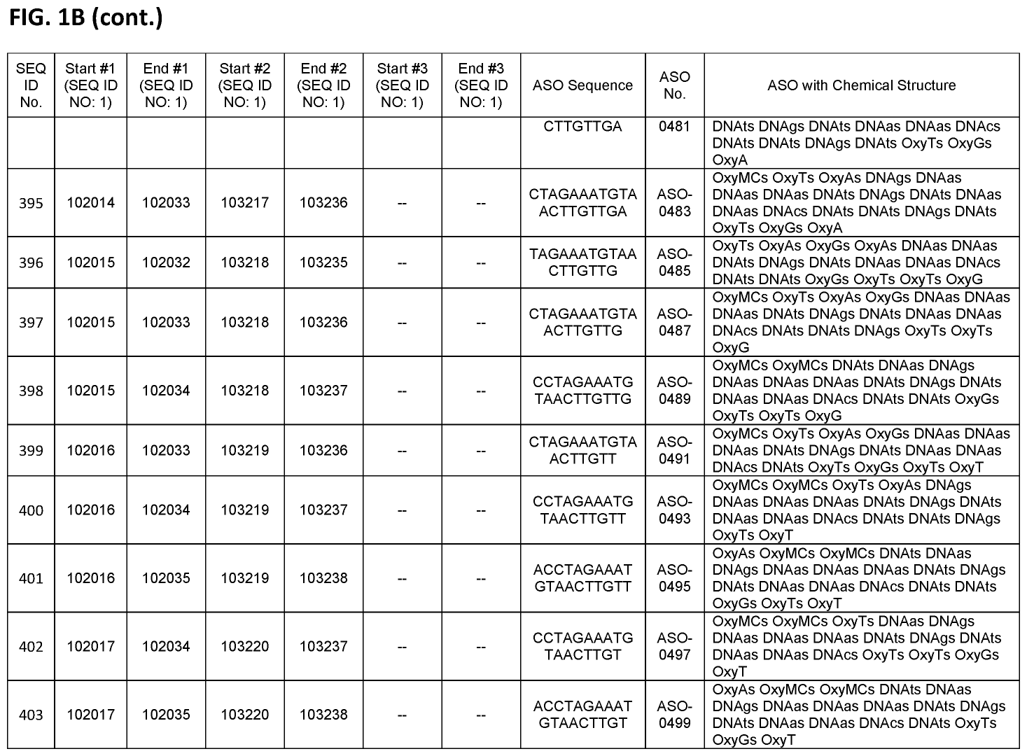

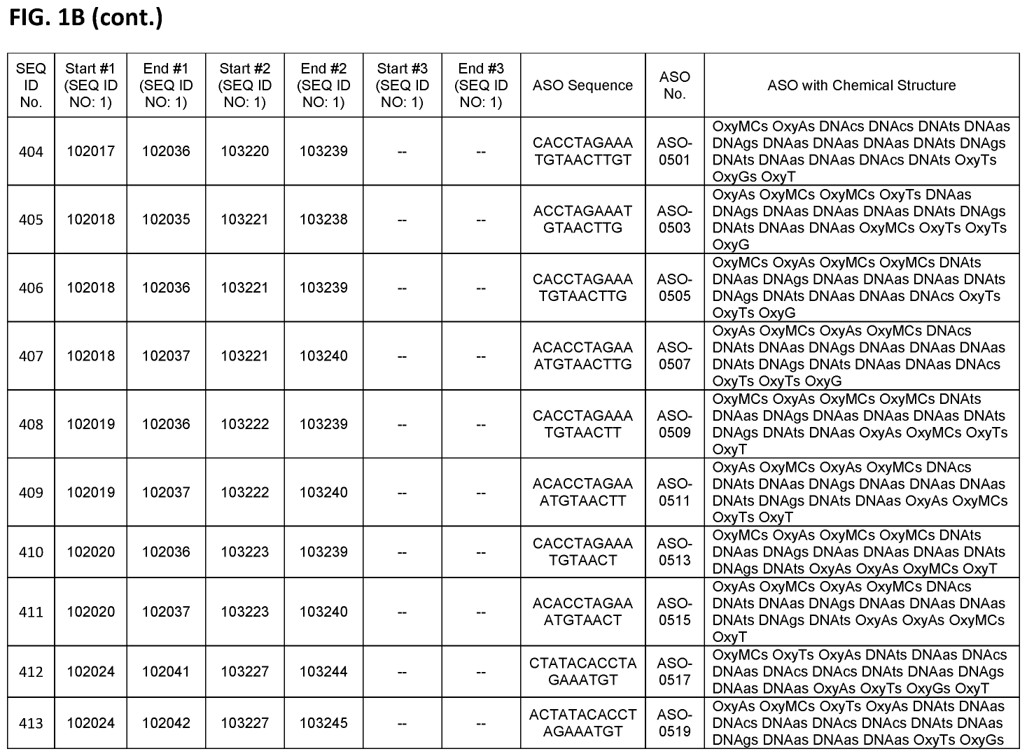

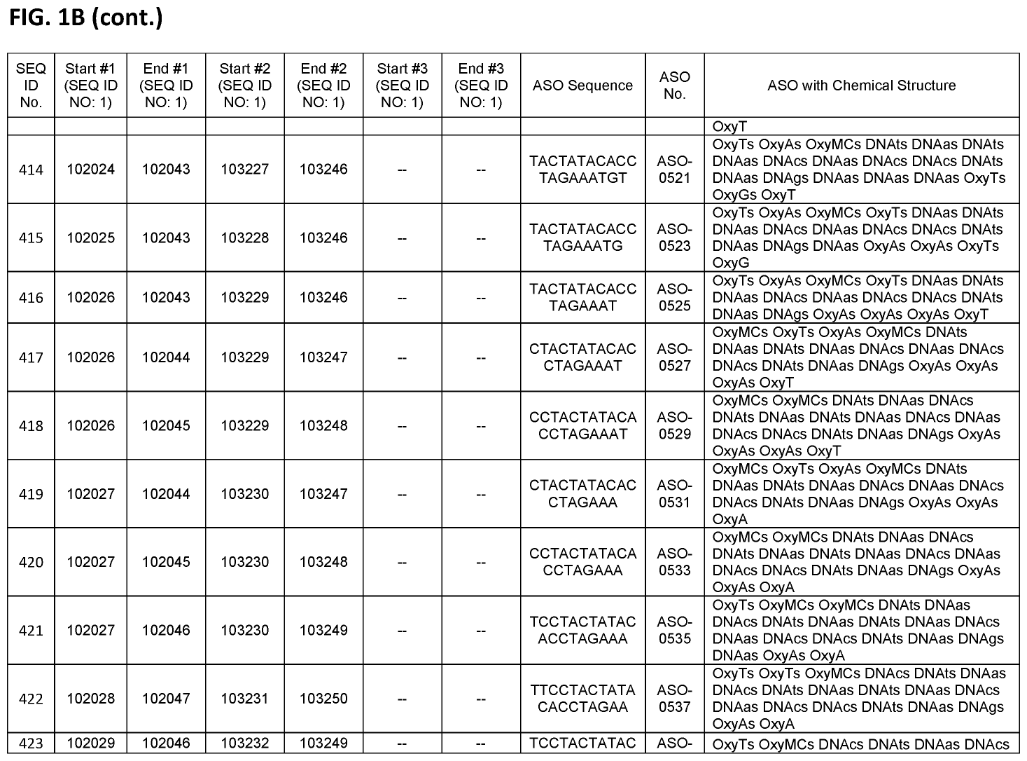

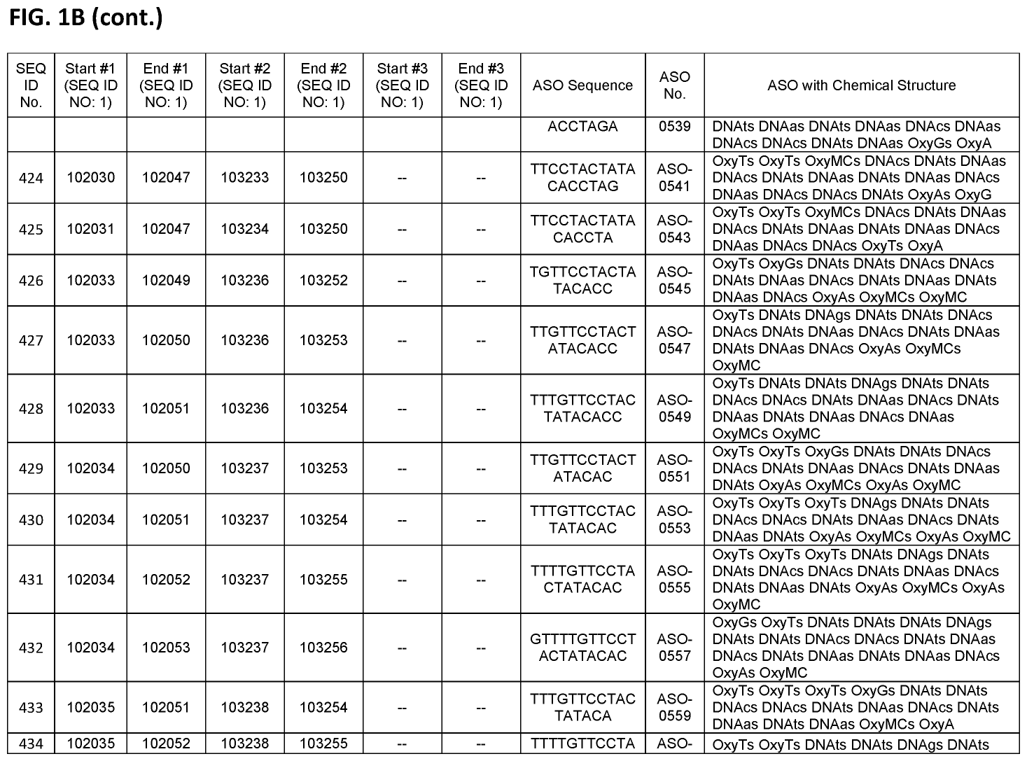

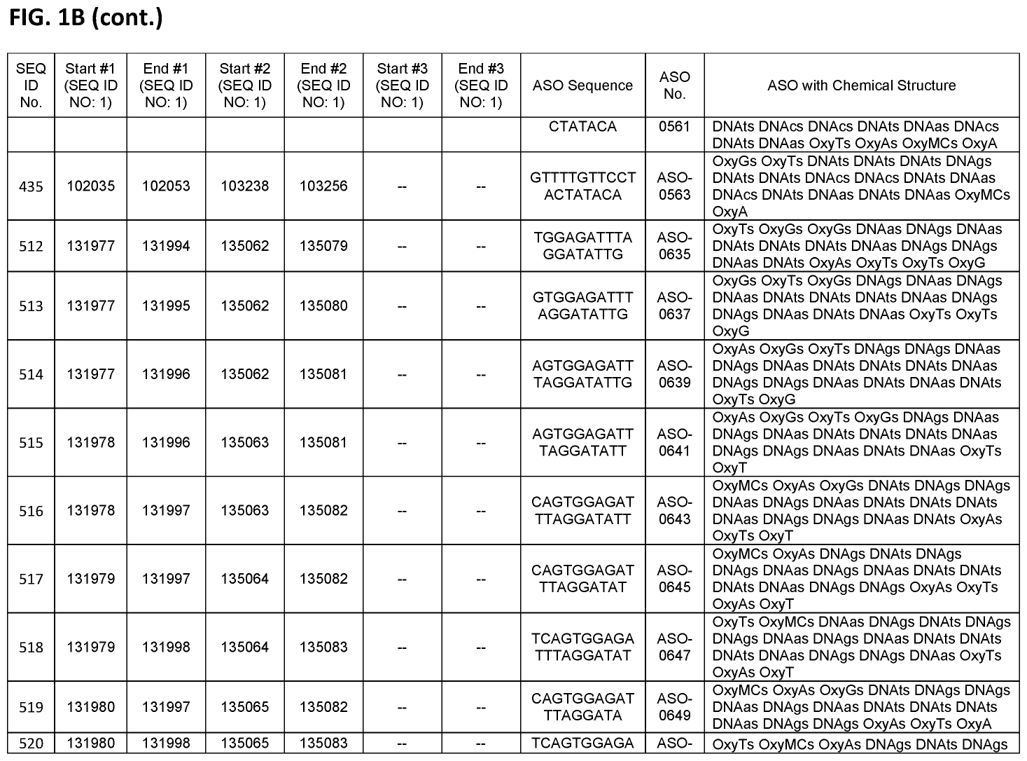

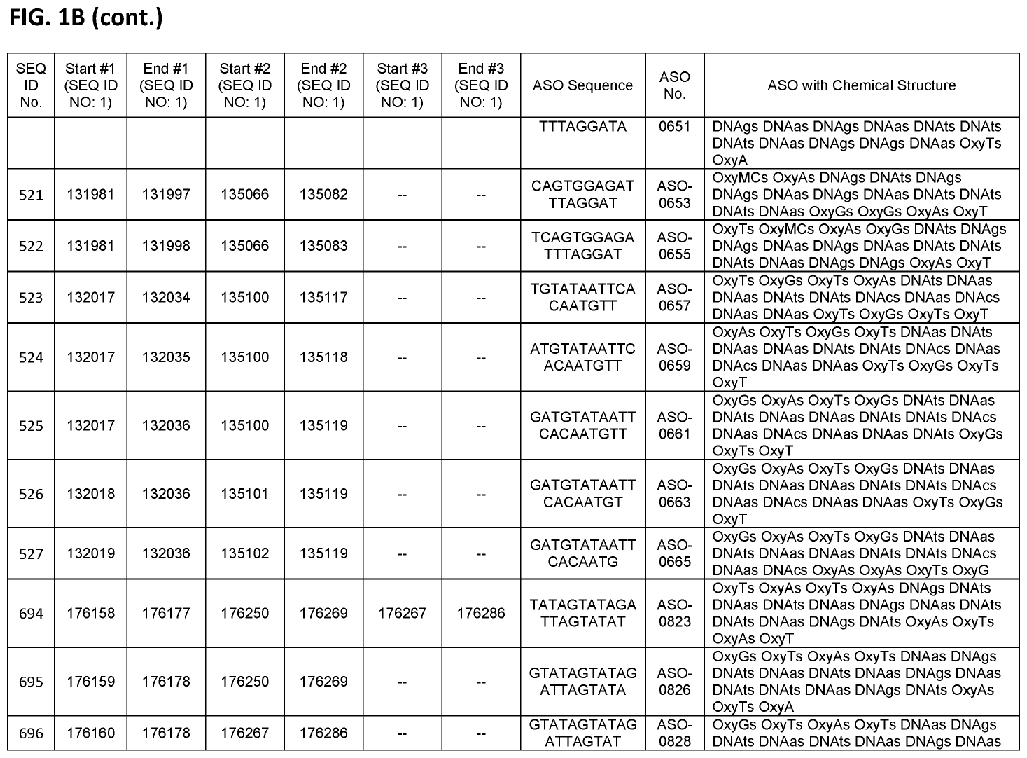

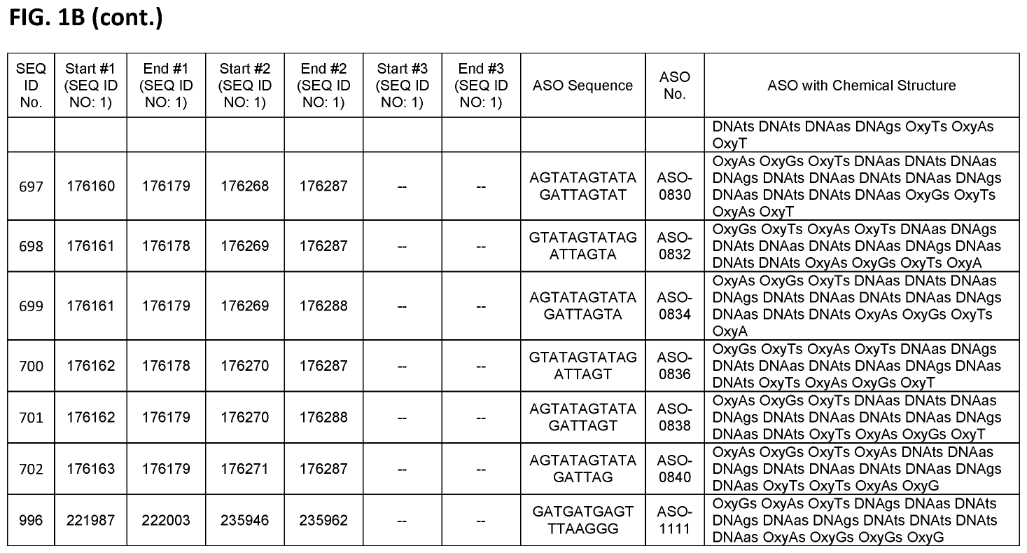

The terms “corresponding to” and “corresponds to,” when referencing two separate nucleic acid or nucleotide sequences can be used to clarify regions of the sequences that correspond or are similar to each other based on homology and/or functionality, although the nucleotides of the specific sequences can be numbered differently. For example, different isoforms of a gene transcript can have similar or conserved portions of nucleotide sequences whose numbering can differ in the respective isoforms based on alternative splicing and/or other modifications. In addition, it is recognized that different numbering systems can be employed when characterizing a nucleic acid or nucleotide sequence (e.g., a gene transcript and whether to begin numbering the sequence from the translation start codon or to include the 5′UTR). Further, it is recognized that the nucleic acid or nucleotide sequence of different variants of a gene or gene transcript can vary. As used herein, however, the regions of the variants that share nucleic acid or nucleotide sequence homology and/or functionality are deemed to “correspond” to one another. For example, a nucleotide sequence of a CAMK2D transcript corresponding to nucleotides X to Y of SEQ ID NO: 1 (“reference sequence”) refers to an CAMK2d transcript sequence (e.g., CAMK2D pre-mRNA or mRNA) that has an identical sequence or a similar sequence to nucleotides X to Y of SEQ ID NO: 1, wherein X is the start site and Y is the end site (as shown in FIGS. 1A and 1B). A person of ordinary skill in the art can identify the corresponding X and Y residues in the CAMK2D transcript sequence by aligning the CAMK2D transcript sequence with SEQ ID NO: 1.

The terms “corresponding nucleotide analog” and “corresponding nucleotide” are intended to indicate that the nucleobase in the nucleotide analog and the naturally occurring nucleotide have the same pairing, or hybridizing, ability. For example, when the 2-deoxyribose unit of the nucleotide is linked to an adenine, the “corresponding nucleotide analog” contains a pentose unit (different from 2-deoxyribose) linked to an adenine.

The term “DES Number” or “DES No.” as used herein refers to a unique number given to a nucleotide sequence having a specific pattern of nucleosides (e.g., DNA) and nucleoside analogs (e.g., LNA). As used herein, the design of an ASO is shown by a combination of upper case letters and lower case letters. For example, DES-0231 refers to an ASO sequence of tacatattatattactcctc (SEQ ID NO: 158) with an ASO design of LLLDDDDDDDDDDDDDLLL (i.e., TACatattatattactcCTC), wherein the L (i.e., upper case letter) indicates a nucleoside analog (e.g., LNA) and the D (i.e., lower case letter) indicates a nucleoside (e.g., DNA).

The annotation of ASO chemistry is as follows Beta-D-oxy LNA nucleotides are designated by OxyB where B designates a nucleotide base such as thymine (T), uridine (U), cytosine (C), 5-methylcytosine (MC), adenine (A) or guanine (G), and thus include OxyA, OxyT, OxyMC, OxyC and OxyG. DNA nucleotides are designated by DNAb, where the lower case b designates a nucleotide base such as thymine (T), uridine (U), cytosine (C), 5-methylcytosine (Mc), adenine (A) or guanine (G), and thus include DNAa, DNAt, DNA and DNAg. The letter M before C or c indicates 5-methylcytosine. The letter “s” indicates a phosphorothioate internucleotide linkage.

The term “ASO Number” or “ASO No.” as used herein refers to a unique number given to a nucleotide sequence having the detailed chemical structure of the components, e.g., nucleosides (e.g., DNA), nucleoside analogs (e.g., beta-D-oxy-LNA), nucleobase (e.g., A, T, G, C, U, or MC), and backbone structure (e.g., phosphorothioate or phosphorodiester). For example, ASO-0231 can refer to OxyTs OxyAs OxyMCs DNAas DNAts DNAas DNAIs DNAts DNAas DNAts DNAas DNAts DNAts DNAas DNAcs DNAts DNAcs OxyMCs OxyTs OxyMC.

“Potency” is normally expressed as an IC₅₀ or EC₅₀, value, in μM, nM or μM unless otherwise stated. Potency can also be expressed in terms of percent inhibition. IC₅₀ is the median inhibitory concentration of a therapeutic molecule. EC₅₀ is the median effective concentration of a therapeutic molecule relative to a vehicle or control (e.g., saline). In functional assays, IC₅₀ is the concentration of a therapeutic molecule that reduces a biological response, e.g., transcription of mRNA or protein expression, by 50% of the biological response that is achieved by the therapeutic molecule. In functional assays, EC₅₀ is the concentration of a therapeutic molecule that produces 50% of the biological response, e.g., transcription of mRNA or protein expression. IC₅₀ or EC₅₀ can be calculated by any number of means known in the art.

As used herein, the term “inhibiting,” e.g., the expression of CAMK2D gene transcript and/or CAMK2D protein refers to the ASO reducing the expression of the CAMK2D gene transcript and/or CAMK2D protein in a cell or a tissue. In some embodiments, the term “inhibiting” refers to complete inhibition (100% inhibition or non-detectable level) of CAMK2D gene transcript or CAMK2D protein. In other embodiments, the term “inhibiting” refers to at least 5%, at least 10%, at least 15%, at least 20%, at least 25%, at least 30%, at least 35%, at least 40%, at least 45%, at least 50%, at least 60%, at least 70%, at least 80%, at least 90%, at least 95% or at least 99% inhibition of CAMK2D gene transcript and/or CAMK2D protein expression in a cell or a tissue.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, bears, and so on.

The term “pharmaceutical composition” refers to a preparation which is in such form as to permit the biological activity of the active ingredient to be effective, and which contains no additional components which are unacceptably toxic to a subject to which the composition would be administered. Such composition can be sterile.

An “effective amount” of an ASO as disclosed herein is an amount sufficient to carry out a specifically stated purpose. An “effective amount” can be determined empirically and in a routine manner, in relation to the stated purpose.

Terms such as “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain embodiments, a subject is successfully “treated” for a disease or condition disclosed elsewhere herein according to the methods provided herein if the patient shows, e.g., total, partial, or transient alleviation or elimination of symptoms associated with the disease or disorder.

### II. Antisense Oligonucleotides

The present disclosure employs antisense oligonucleotides (ASOs) for use in modulating the function of nucleic acid molecules encoding mammalian CAMK2D, such as the CAMK2D nucleic acid, e.g., CAMK2D transcript, including CAMK2D pre-mRNA, and CAMK2D mRNA, or naturally occurring variants of such nucleic acid molecules encoding mammalian CAMK2D. The term “ASO” in the context of the present disclosure, refers to a molecule formed by covalent linkage of two or more nucleotides (i.e., an oligonucleotide).

The ASO comprises a contiguous nucleotide sequence of from about 10 to about 30, such as 10-20, 14-20, 16-20, or 15-25, nucleotides in length. The terms “antisense ASO,” “antisense oligonucleotide,” and “oligomer” as used herein are interchangeable with the term “ASO.”

A reference to a SEQ ID number includes a particular nucleobase sequence, but does not include any design or full chemical structure. Furthermore, the ASOs disclosed in the figures herein show a representative design, but are not limited to the specific design shown in the Figures unless otherwise indicated. Herein, a single nucleotide (unit) can also be referred to as a monomer or unit. When this specification refers to a specific ASO number, the reference includes the sequence, the specific ASO design, and the chemical structure. When this specification refers to a specific DES number, the reference includes the sequence and the specific ASO design. For example, when a claim (or this specification) refers to SEQ ID NO: 158, it includes the nucleotide sequence of tacatattatattactcctc only. When a claim (or the specification) refers to DES-0231, it includes the nucleotide sequence of tacatattatattactcctc with the ASO design of TACatattatattactcCTC. Alternatively, the design of ASO-0231 can also be written as SEQ ID NO: 158, wherein each of the first nucleotide, the second nucleotide, the third nucleotide, the 18^{th} nucleotide, the 19^{th} nucleotide, and the 20^{th} nucleotide from the 5′ end is a modified nucleotide, e.g., LNA, and each of the other nucleotides is a non-modified nucleotide (e.g., DNA). The ASO number includes the sequence and the ASO design, as well as the specific details of the ASO. Therefore, for instance, ASO-0231 referred to in this application indicates OxyTs OxyAs OxyMCs DNAas DNAts DNAas DNAts DNAts DNAas DNAts DNAas DNAts DNAts DNAas DNAcs DNAts DNAcs OxyMCs OxyTs OxyMC, wherein “s” indicates phosphorothioate linkage.

In various embodiments, the ASO of the disclosure does not comprise RNA (units). In some embodiments, the ASO comprises one or more DNA units. In one embodiment, the ASO according to the disclosure is a linear molecule or is synthesized as a linear molecule. In some embodiments, the ASO is a single stranded molecule, and does not comprise short regions of, for example, at least 3, 4 or 5 contiguous nucleotides, which are complementary to equivalent regions within the same ASO (i.e. duplexes)—in this regard, the ASO is not (essentially) double stranded. In some embodiments, the ASO is essentially not double stranded. In some embodiments, the ASO is not a siRNA. In various embodiments, the ASO of the disclosure can consist entirely of the contiguous nucleotide region. Thus, in some embodiments the ASO is not substantially self-complementary.

In other embodiments, the present disclosure includes fragments of ASOs. For example, the disclosure includes at least one nucleotide, at least two contiguous nucleotides, at least three contiguous nucleotides, at least four contiguous nucleotides, at least five contiguous nucleotides, at least six contiguous nucleotides, at least seven contiguous nucleotides, at least eight contiguous nucleotides, or at least nine contiguous nucleotides of the ASOs disclosed herein. Fragments of any of the sequences disclosed herein are contemplated as part of the disclosure.

II.A. The Target

Suitably the ASO of the disclosure is capable of down-regulating (e.g., reducing or removing) expression of the CAMK2D mRNA or protein. In this regard, the ASO of the disclosure can affect indirect inhibition of CAMK2D protein through the reduction in CAMK2D mRNA levels, typically in a mammalian cell, such as a human cell, such as a cardiocyte. In particular, the present disclosure is directed to ASOs that target one or more regions of the CAMK2D pre-mRNA (e.g., intron regions, exon regions, and/or exon-intron junction regions). Unless indicated otherwise, the term “CAMK2D,” as used herein, can refer to CAMK2D from one or more species (e.g., humans, non-human primates, dogs, cats, guinea pigs, rabbits, rats, mice, horses, cattle, and bears).

Calcium/calmodulin-dependent protein kinase type II delta (CAMK2D) is also known as CaM kinase II subunit delta and CamK-II subunit delta. Synonyms of CAMK2D are known and include CaMKIIδ or CAMKD. The sequence for the human CAMK2D gene can be found under publicly available GenBank Accession Number NC_000004.12. The sequence for the human CAMK2D pre-mRNA transcript (SEQ ID NO: 1) corresponds to the reverse complement of residues 113,451,032-113,761,927 of NC_000004.12. The CAMK2D mRNA sequence (GenBank Accession No. NM_001221.3) is provided in SEQ ID NO: 2, except that the nucleotide “t” in SEQ ID NO: 2 is shown as “u” in the mRNA. The sequence for human CAMK2D protein can be found under publicly available Accession Numbers: Q13557 (canonical sequence, SEQ ID NO: 3), A8MVS8, Q52PK4, Q59G21, Q8N553, Q9UGH6, Q9UQE9, each of which is incorporated by reference herein in its entirety.

Natural variants of the human CAMK2D gene product are known. For example, natural variants of human CAMK2D protein can contain one or more amino acid substitutions selected from: D167E, Q463E, and T493I, and any combinations thereof. Additional variants of human CAMK2D protein resulting from alternative splicing are also known in the art. CAMK2D Isoform Delta 3 (identifier: Q13557-3 at UniProt) differs from the canonical sequence (SEQ ID NO: 3) as follows: 328-328: K→KKRKSSSSVQMM. The sequence of CAMK2D Isoform Delta 4 (identifier: Q13557-4) differs from the canonical sequence (SEQ ID NO: 3) as follows: 328-328: K→KINNKANVVTSPKENIPTPAL. The sequence of CAMK2D Isoform Delta 6 (identifier: Q13557-8) differs from the canonical sequence (SEQ ID NO: 3) as follows: 479-499: Missing. The sequence of CAMK2D Isoform Delta 7 (identifier: Q13557-9) differs from the canonical sequence (SEQ ID NO: 3) as follows: 328-328: K→KKRKSSSSVQMM and 479-499: Missing. The sequence of CAMK2D Isoform Delta 8 (identifier: Q13557-5) differs from the canonical sequence (SEQ ID NO: 3) as follows: 328-328: K→KINNKANVVTSPKENIPTPAL and 479-499: Missing. The sequence of CAMK2D Isoform Delta 9 (identifier: Q13557-6) differs from the canonical sequence (SEQ ID NO: 3) as follows: 329-329: E→EPQTTVIHNPDGNKE. The sequence of CAMK2D Isoform Delta 10 (identifier: Q13557-10) differs from the canonical sequence (SEQ ID NO: 3) as follows: 329-329: E→EPQTTVIHNPDGNKE and 479-499: Missing. The sequence of CAMK2D Isoform Delta 11 (identifier: Q13557-11) differs from the canonical sequence (SEQ ID NO: 3) as follows: 328-328: K→KKRKSSSSVQMMEPQTTVIHNPDGNK. The sequence of CAMK2D Isoform Delta 12 (identifier: Q13557-12) differs from the canonical sequence (SEQ ID NO: 3) as follows: 478-478: K→N and 479-499: Missing Therefore, the ASOs of the present disclosure can be designed to reduce or inhibit expression of the natural variants of the CAMK2D protein.

An example of a target nucleic acid sequence of the ASOs is CAMK2D pre-mRNA. SEQ ID NO: 1 represents a human CAMK2D genomic sequence (i.e., reverse complement of nucleotides 113,451,032 to 113,761,927 of GenBank Accession No. NC_000004.12). SEQ ID NO: 1 is identical to a CAMK2D pre-mRNA sequence except that nucleotide “t” in SEQ ID NO: 1 is shown as “u” in pre-mRNA. In certain embodiments, the “target nucleic acid” comprises an intron of a CAMK2D protein-encoding nucleic acids or naturally occurring variants thereof, and RNA nucleic acids derived therefrom, e.g., pre-mRNA. In other embodiments, the target nucleic acid comprises an exon region of a CAMK2D protein-encoding nucleic acids or naturally occurring variants thereof, and RNA nucleic acids derived therefrom, e.g., pre-mRNA. In yet other embodiments, the target nucleic acid comprises an exon-intron junction of a CAMK2D protein-encoding nucleic acids or naturally occurring variants thereof, and RNA nucleic acids derived therefrom, e.g. pre-mRNA. In some embodiments, for example when used in research or diagnostics the “target nucleic acid” can be a cDNA or a synthetic oligonucleotide derived from the above DNA or RNA nucleic acid targets. The human CAMK2D protein sequence encoded by the CAMK2D pre-mRNA is shown as SEQ ID NO: 3. In other embodiments, the target nucleic acid comprises an untranslated region of a CAMK2D protein-encoding nucleic acids or naturally occurring variants thereof, e.g., 5′ UTR, 3′ UTR, or both.

In some embodiments, an ASO of the disclosure hybridizes to a region within the introns of a CAMK2D transcript, e.g., SEQ ID NO: 1. In certain embodiments, an ASO of the disclosure hybridizes to a region within the exons of a CAMK2D transcript, e.g., SEQ ID NO: 1. In other embodiments, an ASO of the disclosure hybridizes to a region within the exon-intron junction of a CAMK2D transcript, e.g., SEQ ID NO: 1. In some embodiments, an ASO of the disclosure hybridizes to a region within a CAMK2D transcript (e.g., an intron, exon, or exon-intron junction), e.g., SEQ ID NO: 1, wherein the ASO has a design according to formula: 5′ A-B-C 3′ as described elsewhere herein (e.g., Section II.G).

In some embodiments, the ASO targets a mRNA encoding a particular isoform of CAMK2D protein (e.g., Isoform Delta 3-12). In some embodiments, the ASO targets all isoforms of CAMK2D protein. In other embodiments, the ASO targets two isoforms (e.g., Isoform Delta 3 and Isoform Delta 7, Isoform Delta 4 and Isoform Delta 8, and Isoform Delta 9 and Isoform Delta 10) of CAMK2D protein.

In some embodiments, the ASO comprises a contiguous nucleotide sequence (e.g., 10 to 30 nucleotides in length) that are complementary to a nucleic acid sequence within a CAMK2D transcript, e.g., a region corresponding to SEQ ID NO: 1. In some embodiments, the ASO comprises a contiguous nucleotide sequence that hybridizes to a nucleic acid sequence, or a region within the sequence, of a CAMK2D transcript (“target region”), wherein the nucleic acid sequence corresponds to nucleotides (i) nucleotides 625-842 of SEQ ID NO: 1; (ii) nucleotides 1,398-59,755 of SEQ ID NO: 1; (iii) nucleotides 61,817-104,725 of SEQ ID NO: 1; (iv) nucleotides 112,162-118,021 of SEQ ID NO: 1; (v) nucleotides 119,440-135,219 of SEQ ID NO: 1; (vi) nucleotides 137,587-157,856 of SEQ ID NO: 1; (vii) nucleotides 159,191-266,174 of SEQ ID NO: 1; and (viii) nucleotides 272,788-310,949 of SEQ ID NO: 1, and wherein, optionally, the ASO has one of the designs described herein (e.g., Section II.G) or a chemical structure shown elsewhere herein (e.g., FIGS. 1A and 1B).

In some embodiments, the target region corresponds to nucleotides 725-742 of SEQ ID NO: 1. In other embodiments, the target region corresponds to nucleotides 1,498-59,655 of SEQ ID NO: 1. In certain embodiments, the target region corresponds to nucleotides 61,917-104,625 of SEQ ID NO: 1. In some embodiments, the target region corresponds to nucleotides 112,262-117,921 of SEQ ID NO: 1. In some embodiments, the target region corresponds to nucleotides 119,540-135,119 of SEQ ID NO: 1. In further embodiments, the target region corresponds to nucleotides 137,687-157,756 of SEQ ID NO: 1. In certain embodiments, the target region corresponds to nucleotides 159,291-266,074 of SEQ ID NO: 1. In some embodiments, the target region corresponds to nucleotides 272,888-310,849 of SEQ ID NO: 1.

In some embodiments, the target region corresponds to nucleotides 725-742 of SEQ ID NO: 1±10, ±20, ±30, ±40, ±50, ±60, ±70, ±80, or ±90 nucleotides at the 3′ end and/or the 5′ end. In other embodiments, the target region corresponds to nucleotides 1,498-59,655 of SEQ ID NO: 1±10, ±20, ±30, ±40, ±50, ±60, ±70, ±80, or ±90 nucleotides at the 3′ end and/or the 5′ end. In certain embodiments, the target region corresponds to nucleotides 61,917-104,625 of SEQ ID NO: 1±10, ±20, ±30, ±40, ±50, ±60, ±70, ±80, or ±90 nucleotides at the 3′ end and/or the 5′ end. In some embodiments, the target region corresponds to nucleotides 112,262-117,921 of SEQ ID NO: 1±10, ±20, ±30, ±40, ±50, ±60, ±70, ±80, or ±90 nucleotides at the 3′ end and/or the 5′ end. In some embodiments, the target region corresponds to nucleotides 119,540-135,119 of SEQ ID NO: 1±10, ±20, ±30, ±40, ±50, ±60, ±70, ±80, or ±90 nucleotides at the 3′ end and/or the 5′ end. In further embodiments, the target region corresponds to nucleotides 137,687-157,756 of SEQ ID NO: 1±10, ±20, ±30, ±40, ±50, ±60, ±70, ±80, or ±90 nucleotides at the 3′ end and/or the 5′ end. In certain embodiments, the target region corresponds to nucleotides 159,291-266,074 of SEQ ID NO: 1±10, ±20, ±30, ±40, ±50, ±60, ±70, ±80, or ±90 nucleotides at the 3′ end and/or the 5′ end. In some embodiments, the target region corresponds to nucleotides 272,888-310,849 of SEQ ID NO: 1±10, ±20, ±30, ±40, ±50, ±60, ±70, ±80, or ±90 nucleotides at the 3′ end and/or the 5′ end.

In some embodiments, the ASO of the present disclosure hybridizes to multiple target regions within the CAMK2D transcript (e.g., pre-mRNA, SEQ ID NO: 1). In some embodiments, the ASO hybridizes to two different target regions within the CAMK2D transcript. In some embodiments, the ASO hybridizes to three different target regions within the CAMK2D transcript. The sequences of exemplary ASOs that hybridizes to multiple target regions, and the start/end sites of the different target regions are provided in FIG. 1B. In some embodiments, the ASOs that hybridizes to multiple regions within the CAMK2D transcript (e.g., pre-mRNA, SEQ ID NO: 1) are more potent (e.g., having lower EC50) at reducing CAMK2D expression compared to ASOs that hybridizes to a single region within the CAMK2D transcript (e.g., pre-mRNA, SEQ ID NO: 1).

In some embodiments, the ASO of the disclosure is capable of hybridizing to the target nucleic acid (e.g., CAMK2D transcript) under physiological condition, i.e., in vivo condition. In some embodiments, the ASO of the disclosure is capable of hybridizing to the target nucleic acid (e.g., CAMK2D transcript) in vitro. In some embodiments, the ASO of the disclosure is capable of hybridizing to the target nucleic acid (e.g., CAMK2D transcript) in vitro under stringent conditions. Stringency conditions for hybridization in vitro are dependent on, inter alia, productive cell uptake, RNA accessibility, temperature, free energy of association, salt concentration, and time (see, e.g., Stanley T Crooke, Antisense Drug Technology: Principles, Strategies and Applications, 2^{nd} Edition, CRC Press (2007)). Generally, conditions of high to moderate stringency are used for in vitro hybridization to enable hybridization between substantially similar nucleic acids, but not between dissimilar nucleic acids. An example of stringent hybridization conditions includes hybridization in 5× saline-sodium citrate (SSC) buffer (0.75 M sodium chloride/0.075 M sodium citrate) for 1 hour at 40° C., followed by washing the sample 10 times in 1×SSC at 40° C. and 5 times in 1×SSC buffer at room temperature. In viva hybridization conditions consist of intracellular conditions (e.g., physiological pH and intracellular ionic conditions) that govern the hybridization of antisense oligonucleotides with target sequences. In view conditions can be mimicked in vitro by relatively low stringency conditions. For example, hybridization can be carried out in vitro in 2×SSC (0.3 M sodium chloride/0.03 M sodium citrate), 0.1% SDS at 37° C. A wash solution containing 4×SSC, 0.1% SDS can be used at 37° C., with a final wash in 1×SSC at 45° C.

In some embodiments, the ASO of the present disclosure is capable of targeting a CAMK2D transcript from one or more species (e.g., humans, non-human primates, dogs, cats, guinea pigs, rabbits, rats, mice, horses, cattle, and bears). In certain embodiments, the ASO disclosed herein is capable of targeting both human and rodent (e.g., mice or rats) CAMK2D transcript. Accordingly, in some embodiments, the ASO is capable of down-regulating (e.g., reducing or removing) expression of the CAMK2D mRNA or protein both in humans and in rodents (e.g., mice or rats).

Sequences of mouse CAMK21) transcript are known in the art. For instance, the sequence for the mouse CAMK2D gene can be found under publicly available GenBank Accession Number NC_000069.6. The sequence for the mouse CAMK2D pre-mRNA transcript corresponds to residues 126,596,354-126,846,326 of NC_000069.6. The sequences for mouse CAMK2D mRNA transcript (both canonical and variants) are known and available as Accession Numbers NM_001025438.2 (canonical sequence), NM_001025439.2, NM_001293663.1, NM_001293664.1, NM_023813.4, NM_001346635.1, NM_001346636.1, NM_001293665.1, XM_006500836.3, XM_006500833.3, XM_006500835.3, XM_017319415.1, XM_006500818.3, XM_017319417.1, XM_017319418.1, XM 017319420.1, NM_001293666.1, XM 006500819.3, XM_017319416.1, XM 006500820.3, XM_006500822.3, XM_006500823.3, XM_006500824.3, XM_017319419.1, XM_006500826.3, XM_006500825.3, XM 006500829.3, BC052894.1, XM_006500831.3, XM 006500832.3, XM_017319422.1, XM_006500834.3, XM 006500839.3, and XM_017319421.1. The sequence of mouse CAMK2D protein can be found under publicly available Accession Numbers: Q6PHZ2 (canonical sequence), Q3UF87, Q3UQH9, Q5DTK4, Q8CACS, and Q9CZE2, each of which is incorporated by reference herein in its entirety. Three isoforms of the mouse CAMK2D protein are known. The sequence of CAMK2D Isoform Delta 6 differs from the canonical sequence as follows: 478-478: K→N and 479-499: Missing. The sequence of CAMK2D Isoform Delta 10 differs from the canonical as follows: 329-329: E→EPQTTVIHNPDGNKE; 478-478: K→N; and 479-499: Missing. The sequence of CAMK2D Isoform Delta 5 differs from the canonical sequence (as follows: 328-328: K→KINNKANVVTSPKENIPTPALEPQTTVIHNPDGNK; 478-478: K→N; and 479-499: Missing.

Sequences of rat CAMK2D transcript are also known in the art. The rat CAMK2D gene can be found under publicly available GenBank Accession Number NC_005101.4. The sequence for the rat CAMK2D pre-mRNA transcript corresponds to residues 230,900,907-231,132,207 of NC_005101.4. The sequences for rat CAMK2D mRNA transcript (both canonical and variants) are known and available as Accession Number NM_012519.2 (canonical sequence), BC107562.1, XM_017590621.1, XM_017590605.1, XM_008761452.1, XM_017590606.1, XM_017590607.1, XM_017590608.1, XM_017590610.1, XM_017590611.1, XM_017590612.1, XM_006233285.3, XM_017590614.1, XM_017590615.1, XM_017590616.1, XM_017590613.1, XM_017590617.1, XM_017590618.1, XM_017590604.1, XM_017590609.1, XM_017590624.1, XM_017590625.1, XM_017590619.1, XM_017590620.1, XM_017590622.1, and XM_017590623.1. The sequence of rat CAMK2D protein can be found under publicly available Accession Numbers' P15791 (canonical sequence), P97915, P97916, Q3B7L0, Q63904, Q63905, Q63906, Q63907, and Q63908, each of which is incorporated by reference herein in its entirety. Six isoforms of rat CAMK2D protein are known. The sequence of CAMK2D Isoform Delta 2 differs from the canonical sequence as follows: 329-362: Missing. The sequence of CAMK2D Isoform Delta 3 differs from the canonical sequence as follows: 329-335: INNKANV→KRKSSSV; 337-359: Missing; and 360-362: GNK→QMM. The sequence of CAMK2D Isoform Delta 4 differs from the canonical sequence as follows: 349-362: Missing. The sequence of CAMK2D Isoform Delta 5 differs from the canonical sequence as follows: 329-362: Missing and 512-533: KPPCIPNGKENFSGGTSLWQNI→N. The sequence of CAMK2D Isoform Delta 6 differs from the canonical sequence as follows: 512-533: KPPCIPNGKENFSGGTSLWQNI→N. The sequence of CAMK2D Isoform Delta 7 differs from the canonical sequence as follows: 349-362: Missing and 512-533: KPPCIPNGKENFSGGTSLWQNI→N.

II.B. ASO Sequences

The ASOs of the disclosure comprise a contiguous nucleotide sequence which corresponds to the complement of a region of CAMK2D transcript, e.g., a nucleotide sequence corresponding to SEQ ID NO: 1.

In certain embodiments, the disclosure provides an ASO from 10-30, such as 10-15 nucleotides, 10-20 nucleotides, or 10-25 nucleotides in length, wherein the contiguous nucleotide sequence has at least about 80%, at least about 85%, at least about 90%, at least about 95%, at least about 96%, at least about 97%, at least about 98%, at least about 99%, or about 100% sequence identity to a region within the complement of a CAMK2D transcript, such as SEQ ID NO: 1 or naturally occurring variant thereof. Thus, for example, the ASO hybridizes to a single stranded nucleic acid molecule having the sequence of SEQ ID NO: 1 or a portion thereof.

The ASO can comprise a contiguous nucleotide sequence which is fully complementary (perfectly complementary) to the equivalent region of a nucleic acid which encodes a mammalian CAMK2D protein (e.g., SEQ ID NO: 1). The ASO can comprise a contiguous nucleotide sequence which is fully complementary (perfectly complementary) to a nucleic acid sequence, or a region within the sequence, corresponding to nucleotides X—Y of SEQ ID NO: 1, wherein X and Y are the start site and the end site, respectively, as shown in FIGS. 1A and 1B.

In some embodiments, the nucleotide sequence of the ASOs of the disclosure or the contiguous nucleotide sequence has at least about 80% sequence identity to a sequence selected from SEQ ID NOs: 4 to 1713 (i.e., the sequences in FIGS. 1A and 1B), such as at least about 80%, at least about 85%, at least about 90%, at least about 91%, at least about 92%, at least about 93%, at least about 94%, at least about 95%, at least about 96% sequence identity, at least about 97% sequence identity, at least about 98% sequence identity, at least about 99% sequence identity, such as about 100% sequence identity (homologous). In some embodiments, the ASO has a design described elsewhere herein (e.g., Section II.G) or a chemical structure shown elsewhere herein (e.g., FIGS. 1A and 1B).

In some embodiments the ASO (or contiguous nucleotide portion thereof) is selected from, or comprises, one of the sequences selected from the group consisting of SEQ ID NOs: 4 to 1713 or a region of at least 10 contiguous nucleotides thereof, wherein the ASO (or contiguous nucleotide portion thereof) can optionally comprise one, two, three, or four mismatches when compared to the corresponding CAMK2D transcript.

In some embodiments, the ASO comprises a sequence selected from the group consisting of SEQ ID NO: 254, SEQ ID NO. 27, SEQ ID NO: 114. SEQ ID NO: 158, SEQ ID NO: 190, SEQ ID NO: 327, SEQ ID NO: 463, SEQ ID NO: 513, SEQ ID NO: 516, SEQ ID NO: 519, SEQ ID NO: 657, SEQ ID NO: 659, SEQ ID NO: 827, SEQ ID NO: 1249, SEQ ID NO: 1326, SEQ ID NO: 1409, SEQ ID NO: 1524, SEQ ID NO: 1530, SEQ ID NO: 1662, and SEQ ID NO: 1676.

In some embodiments, the ASO comprises a sequence selected from the group consisting of SEQ ID NO: 55, SEQ ID NO: 61, SEQ ID NO: 63, SEQ ID NO: 71, SEQ ID NO: 75, SEQ ID NO: 79, SEQ ID NO: 84, SEQ ID NO: 85, SEQ ID NO: 92, SEQ ID NO: 102, SEQ ID NO: 105, SEQ ID NO: 128, SEQ ID NO: 130, SEQ ID NO: 133, SEQ ID NO: 138, SEQ ID NO: 161, SEQ ID NO: 178, SEQ ID NO: 180, SEQ ID NO: 186, SEQ ID NO: 195, SEQ ID NO: 200, SEQ ID NO: 202, SEQ ID NO: 234, SEQ ID NO: 264, SEQ ID NO: 387, SEQ ID NO: 390, SEQ ID NO: 396, SEQ ID NO: 441, SEQ ID NO: 446, SEQ ID NO: 457, SEQ ID NO: 467, SEQ ID NO: 523, SEQ ID NO: 524, SEQ ID NO: 636, SEQ ID NO: 640, SEQ ID NO: 700, SEQ ID NO: 740, SEQ ID NO: 832, SEQ ID NO: 965, SEQ ID NO: 1015, SEQ ID NO: 1065, SEQ ID NO: 1071, SEQ ID NO: 1155, SEQ ID NO: 1475, SEQ ID NO: 1508, SEQ ID NO: 1685, SEQ ID NO: 1686, SEQ ID NO: 1687, SEQ ID NO: 1688, and SEQ ID NO: 1690.

In some embodiments, the ASOs of the disclosure bind to the target nucleic acid sequence (e.g., CAMK2D transcript) and are capable of inhibiting or reducing expression of the CAMK2D transcript by at least 10% or 20% compared to the normal (i.e., control) expression level in the cell, e.g., at least about 30%, at least about 40%, at least about 50%, at least about 60%, at least about 70%, at least about 80%, at least about 90%, at least about 95%, at least about 96%, at least about 97%, at least about 98%, at least about 99%, or about 100% compared to the normal expression level (e.g., expression level in cells that have not been exposed to the ASO).

In some embodiments, the ASOs of the disclosure are capable of reducing expression of CAMK2D mRNA in vitro by at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, or about 100% in HEK293 cells when the cells are in contact with 25 μM of the ASO compared to HEK293 cells that are not in contact with the ASO (e.g., contact with saline).

In some embodiments, the ASOs of the disclosure are capable of reducing expression of CAMK2D mRNA in vitro by at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, or about 100% in human inducible pluripotent stem cell-derived cardiomyocytes (hiPSC-CM) cells when the cells are in contact with 500 nM of the ASO compared to hiPSC-CM cells that are not in contact with the ASO (e.g., contact with saline).

In certain embodiments, the ASO of the disclosure has at least one property selected from the group consisting of: (i) reducing an mRNA level encoding CAMK2D in Inducible Pluripotent Stem Cell-Derived Cardiomyocytes (hiPSC-CM); (ii) reducing a protein level of CAMK2D in hiPSC-CM; (iii) reducing, ameliorating, or treating one or more symptoms of a cardiovascular disease or disorder, and (iv) any combination thereof.

In some embodiments, the ASO can tolerate 1, 2, 3, or 4 (or more) mismatches, when hybridizing to the target sequence and still sufficiently bind to the target to show the desired effect, i.e., down-regulation of the target mRNA and/or protein. Mismatches can, for example, be compensated by increased length of the ASO nucleotide sequence and/or an increased number of nucleotide analogs, which are disclosed elsewhere herein.

In some embodiments, the ASO of the disclosure comprises no more than 3 mismatches when hybridizing to the target sequence. In other embodiments, the contiguous nucleotide sequence comprises no more than 2 mismatches when hybridizing to the target sequence. In other embodiments, the contiguous nucleotide sequence comprises no more than 1 mismatch when hybridizing to the target sequence.

II.C. ASO Length

The ASOs can comprise a contiguous nucleotide sequence of a total of 10, 11, 12, 13, 14, 15, 16, 17, 18, 19, 20, 21, 22, 23, 24, 25, 26, 27, 28, 29, or 30 contiguous nucleotides in length. It should be understood that when a range is given for an ASO, or contiguous nucleotide sequence length, the range includes the lower and upper lengths provided in the range, for example from (or between) 10-30, includes both 10 and 30.

In some embodiments, the ASOs comprise a contiguous nucleotide sequence of a total of about 14-20, 14, 15, 16, 17, 18, 19, or 20 contiguous nucleotides in length.

II.D. Nucleosides and Nucleoside Analogs

In one aspect of the disclosure, the ASOs comprise one or more non-naturally occurring nucleoside analogs. “Nucleoside analogs” as used herein are variants of natural nucleosides, such as DNA or RNA nucleosides, by virtue of modifications in the sugar and/or base moieties. Analogs could in principle be merely “silent” or “equivalent” to the natural nucleosides in the context of the oligonucleotide, i.e. have no functional effect on the way the oligonucleotide works to inhibit target gene expression. Such “equivalent” analogs can nevertheless be useful if, for example, they are easier or cheaper to manufacture, or are more stable to storage or manufacturing conditions, or represent a tag or label. In some embodiments, however, the analogs will have a functional effect on the way in which the ASO works to inhibit expression; for example by producing increased binding affinity to the target and/or increased resistance to intracellular nucleases and/or increased ease of transport into the cell. Specific examples of nucleoside analogs are described by e.g. Freier & Altmann; Nucl. Acid Res., 1997, 25, 4429-4443 and Uhlmann; Curr. Opinion in Drug Development, 2000, 3(2), 293-213, and in Scheme 1. The ASOs of the present disclosure can contain more than one, more than two, more than three, more than four, more than five, more than six, more than seven, more than eight, more than nine, more than 10, more than 11, more than 12, more than 13, more than 14, more than 15, more than 16, more than 18, more than 19, or more than 20 nucleoside analogs. In some embodiments, the nucleoside analogs in the ASOs are the same. In other embodiments, the nucleoside analogs in the ASOs are different. The nucleotide analogs in the ASOs can be any one of or combination of the following nucleoside analogs.

II.D.1. Nucleobase

The term nucleobase includes the purine (e.g., adenine and guanine) and pyrimidine (e.g., uracil, thymine and cytosine) moiety present in nucleosides and nucleotides which form hydrogen bonds in nucleic acid hybridization. In the context of the present disclosure, the term nucleobase also encompasses modified nucleobases which may differ from naturally occurring nucleobases, but are functional during nucleic acid hybridization. In some embodiments, the nucleobase moiety is modified by modifying or replacing the nucleobase. In this context, “nucleobase” refers to both naturally occurring nucleobases such as adenine, guanine, cytosine, thymidine, uracil, xanthine and hypoxanthine, as well as non-naturally occurring variants. Such variants are for example described in Hirao et al., (2012) Account of Chemical Research vol 45 page 2055 and Bergstrom (2009) Current Protocols in Nucleic Acid Chemistry Suppl. 37 1.4.1.

In a some embodiments, the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl-cytosine, 5-thiozolo-cytosine, 5-propynyl-cytosine, 5-propynyl-uracil, 5-bromouracil, 5-thiazolo-uracil, 2-thio-uracil, 2′thio-thymine, inosine, diaminopurine, 6-aminopurine, 2-aminopurine, 2,6-diaminopurine, and 2-chloro-6-aminopurine.

The nucleobase moieties may be indicated by the letter code for each corresponding nucleobase, e.g., A, T, G, C, or U, wherein each letter may optionally include modified nucleobases of equivalent function. For example, in the exemplified oligonucleotides, the nucleobase moieties are selected from A, T, G, C, and 5-methyl-cytosine. Optionally, for LNA gapmers, 5-methyl-cytosine LNA nucleosides may be used.

II.D.2. Sugar Modification

The ASO of the disclosure can comprise one or more nucleosides which have a modified sugar moiety, i.e. a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradical bridge between the C2′ and C4′ carbons on the ribose ring (LNA), or an unlinked ribose ring which typically lacks a bond between the C2′ and C3′ carbons (e.g., UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in RNA nucleosides. Substituents may, for example be introduced at the 2′, 3′, 4′, or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity.

II.D.2a 2′ Modified Nucleosides

A 2′ sugar modified nucleoside is a nucleoside which has a substituent other than H or —OH at the 2′ position (2′ substituted nucleoside) or comprises a 2′ linked biradical, and includes 2′ substituted nucleosides and LNA (2′-4′ biradical bridged) nucleosides. For example, the 2′ modified sugar may provide enhanced binding affinity (e.g., affinity enhancing 2′ sugar modified nucleoside) and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-Fluro-DNA, arabino nucleic acids (ANA), and 2′-Fluoro-ANA nucleoside. For further examples, please see. e.g., Freier & Altmann; Nucl. Acid Res., 1997, 25, 4429-4443; Uhlmann, Curr. Opinion in Drug Development, 2000, 3(2), 293-213; and Deleavey and Damha, Chemistry and Biology 2012, 19,937. Below are illustrations of some 2′ substituted modified nucleosides.

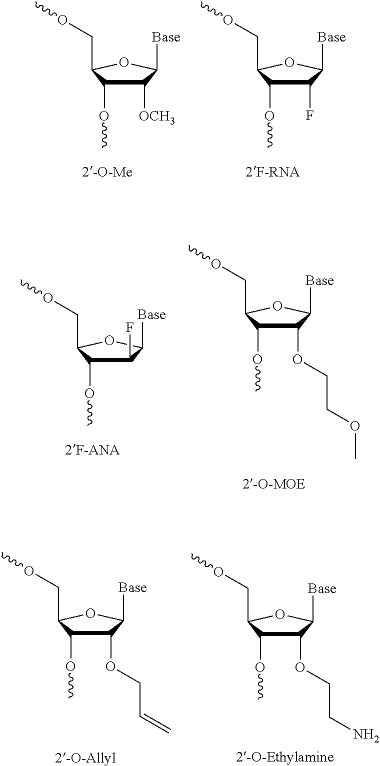

II.D2.b Locked Nucleic Acid Nucleosides (LNA).

LNA nucleosides are 2′-sugar modified nucleosides which comprise a linker group (referred to as a biradical or a bridge) between C2′ and C4′ of the ribose sugar ring of a nucleoside (i.e., 2′-4′ bridge), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226. WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al., J. Org. Chem. 2010, Vol 75(5) pp. 1569-81, and Mitsuoka et al., Nucleic Acids Research 2009, 37(4), 1225-1238.

The 2′-4′ bridge comprises 1 to 4 bridging atoms and is in particular of formula —X—Y— wherein

X is oxygen, sulfur, —CR^{a}R^{b}—, —C(R^{a})═C(R^{b}), —C(═CR^{a}R^{b})—, —C(R^{a})═N, —Si(R^{a})2-, —SO2-, —NR^{a}—; —O—NR^{a}—, —NR^{a}—O—, >C=J, Se; -cPr—, —O—NR^{a}—, NR^{a}—CR^{a}R^{b}—, —N(R^{a})—O—, or —O—CR^{a}R^{b};

Y is oxygen, sulfur, —(CR^{a}R^{b})ₙ—, —CR^{a}R^{b}—O—CR^{a}R^{b}—, —C(R^{a})C(R^{b}), —C(R^{a})═N, —Si(R^{b})2-, —SO2-, —NR^{a}—, or >C═Se; -cPr—, —O—NR^{a}—, —O—CR^{a}R^{b}—, or NR^{a}—CR^{a}R^{b}—; wherein n is 1 or 2;

with the proviso that —X—Y— is not —O—O—, Si(R^{a})₂—Si(R^{a})₂—, —SO₂—SO₂—, —C(R^{a})═C(R^{b})—C(R^{a})═C(R^{b}), —C(R^{a})═N—C(R^{a})═N—, —C(R^{a})═N—C(R^{a})═(R^{b}), —C(R^{a})═C(R^{b})—C(R^{a})═N—, or —Se—Se—;

J is oxygen, sulfur, CH₂, or ═N(R^{a});

R^{a} and R^{b} are independently selected from hydrogen, halogen, hydroxyl, cyano, thiohydroxyl, optionally substituted alkyl, optionally substituted alkenyl, optionally substituted alkynyl, optionally substituted alkoxy, alkoxyalkyl, alkenyloxy, carboxyl, alkoxycarbonyl, alkylcarbonyl, formyl, aryl, heterocycle, amino, alkylamino, carbamoyl, alkylaminocarbonyl, aminoalkylaminocarbonyl, alkylaminoalkylaminocarbonyl, alkylcarbonylamino, carbamido, alkanoyloxy, sulfone alkylsulfonyloxy, nitro, azido, thiolsulfidealkylsulfanyl, aryloxycarbonyl, aryloxy, arylcarbonyl, heteroaryl, heteroaryloxycarbonyl, heteroaryloxy, heteroarylcarbonyl, —OC(═X^{a})R^{c}, —OC(═X^{a})NR^{c}R^{d} and —NR^{e}C(═X^{a})NR^{c}R^{d}; or two geminal R^{a} and R^{b} together form optionally substituted methylene; wherein substituted alkyl, substituted alkenyl, substituted alkynyl, substituted alkoxy and substituted methylene are alkyl, alkenyl, alkynyl and methylene substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl, formyl, heterocycle, aryl, and heteroaryl;

X^{a} is oxygen, sulfur or —NRc;
R^{c}, R^{d}, and R^{e} are independently hydrogen or alkyl; and
n is 1, 2 or 3.

In some embodiments, X is oxygen, sulfur, —NR^{a}—, —CR^{a}R^{b}— or —C(═CR^{a}R^{b})—, particularly oxygen, sulfur, —NH—, —CH— or —C(═CH₂)—, more particularly oxygen.

In some embodiments, Y is —CR^{a}R^{b}—, —CR^{a}R^{b}—CR^{a}R^{b}— or —CR^{a}R^{b}—CR^{a}R^{b}—CR^{a}R^{b}—, particularly —CH₂—CHCH₃—, —CHCH₃—CH—, CH₂—CH₂— or —CH₂—CH₂—CH₂—.

In some embodiments, —X—Y— is —O—(CR^{a}R^{b})ₙ—, —S—CR^{a}R^{b}—, —N(R^{a})CR^{a}R^{b}—, —CR^{a}R^{b}—CR^{a}R^{b}—, —O—CR^{a}R^{b}—O—CR^{a}R^{b}—, —CR^{a}R^{b}—O—CR^{a}R^{b}—, —C(═CR^{a}R^{b})—CR^{a}R^{b}—, —N(R^{a})CR^{a}R^{b}—, —O—N(R^{a})CR^{a}R^{b}—, or —N(R^{a})—O—CR^{a}R^{b}—.

In some embodiments, R^{a} and R^{b} are independently selected from the group consisting of hydrogen, halogen, hydroxyl, alkyl and alkoxyalkyl, in particular, hydrogen, alkyl and alkoxyalkyl.

In some embodiments, R^{a} and R^{b} are independently selected from the group consisting of hydrogen, halogen, such as fluoro, hydroxyl, methyl and —CH₂—O—CH₃, in particular, hydrogen, methyl and —CH₂—O—CH₃.

In some embodiments, R^{a} is hydrogen or alkyl, in particular, hydrogen or methyl.

In some embodiments, R^{b} is hydrogen or alkyl, in particular hydrogen or methyl. In some embodiments, one or both of R^{a} and R^{b} are hydrogen. In certain embodiments, only one of R^{a} and R^{b}is hydrogen. In some embodiments, one of R^{a} and R^{b} is methyl and the other one is hydrogen. In other embodiments, R^{a} and R^{b} are both methyl at the same time.

In a particular embodiment of the invention, —X—Y— is —O—CH₂—, —S—CH₂—, —S—CH(CH₃)—, —NH—CH₂—, —O—CH₂CH₂—, —O—CH(CH₂—O—CH₃)—, —O—CH(CH₂CH₃)—, —O—CH(CH₃)—, —O—CH₂—, —O—CH₂—, —O—CH₂—O—CH₃, —CH₂—O—CH₂—, —C(═CH₂)CH₂—, —C(═CH₂)CH(CH₃)—, —N(—O—CH₃)— or —N(CH₃)—;

In some embodiments, —X—Y— is —O—CR^{a}R^{b}— wherein R^{a} and R^{b} are independently selected from the group consisting of hydrogen, alkyl and alkoxyalkyl, in particular, hydrogen, methyl and —CH₂—O—CH₃.

In some embodiments, —X—Y— is —O—CH₂— or —O—CH(CH₃)—, particularly —O—CH₂—.

The 2′-4′ bridge can be positioned either below the plane of the ribose ring (beta-D-configuration), or above the plane of the ring (alpha-L-configuration), as illustrated in formula (A) and formula (B) respectively.

In some embodiments, the modified nucleoside or the LNA nucleosides of the ASO of the disclosure has a general structure of the formula II or III:

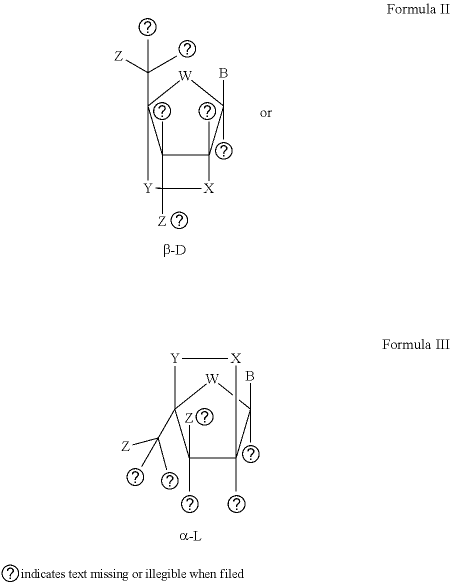

wherein
W is selected from —O—, —S—, —N(R^{a})—, —C(R^{a}R^{b})—, in particular —O—;
B is a nucleobase or a modified nucleobase moiety;
Z is an internucleoside linkage to an adjacent nucleoside or a 5′-terminal group;
Z* is an internucleoside linkage to an adjacent nucleoside or a 3′-terminal group;
R¹, R², R³, R⁵ and R⁵* are independently selected from hydrogen, halogen, alkyl, alkenyl, alkynyl, hydroxy, alkoxy, alkoxyalkyl, alkenyloxy, carboxyl, alkoxycarbonyl, alkylcarbonyl, formyl, azide, heterocycle and aryl; and
X, Y, R^{a} and R^{b} are as defined herein.

In some embodiments, —X—Y—, R^{a} is hydrogen or alkyl, in particular hydrogen or methyl. In some embodiments of —X—Y—, R^{b} is hydrogen or alkyl, in particular hydrogen or methyl. In other embodiments of —X—Y—, one or both of R^{a} and R^{b} are hydrogen. In further embodiments of —X—Y—, only one of R^{a} and R^{b} is hydrogen. In some embodiments of —X—Y—, one of R^{a} and R^{b} is methyl and the other one is hydrogen. In certain embodiments of —X—Y—, R^{a} and R^{b} are both methyl at the same time.

In some embodiments, —X—, R^{a} is hydrogen or alkyl, in particular hydrogen or methyl. In some embodiments of —X—, R^{b} is hydrogen or alkyl, in particular hydrogen or methyl. In other embodiments of —X—, one or both of R^{a} and R^{b} are hydrogen. In certain embodiments of —X—, only one of R^{a} and R^{b} is hydrogen. In certain embodiments of —X—, one of R^{a} and R^{b} is methyl and the other one is hydrogen. In other embodiments of —X—, R^{a} and R^{b} are both methyl at the same time.

In some embodiments, —Y—, R^{a} is hydrogen or alkyl, in particular hydrogen or methyl. In certain embodiments of —Y—, R^{a} is hydrogen or alkyl, in particular hydrogen or methyl. In other embodiments of —Y—, one or both of R^{a} and R^{b} are hydrogen. In some embodiments of —Y—, only one of R^{a} and R^{b} is hydrogen. In other embodiments of —Y—, one of R^{a} and R^{b} is methyl and the other one is hydrogen. In some embodiments of —Y—, R^{a} and R^{b} are both methyl at the same time.

In some embodiments, R¹, R², R³, R⁵ and R⁵* are independently selected from hydrogen and alkyl, in particular hydrogen and methyl.

In some embodiments, R¹, R², R³, R⁵ and R⁵* are all hydrogen at the same time.

In some embodiments, R¹, R², R³, are all hydrogen at the same time, one of R⁵ and R^{5′} is hydrogen and the other one is as defined above, in particular alkyl, more particularly methyl.

In some embodiments, R¹, R², R³, are all hydrogen at the same time, one of R⁵ and R⁵* is hydrogen and the other one is azide.

In some embodiments, —X—Y— is —O—CH₂—, W is oxygen and R¹, R², R³, R⁵ and R⁵* are all hydrogen at the same time. Such LNA nucleosides are disclosed in WO 99/014226, WO 00/66604, WO 98/039352 and WO 2004/046160, which are all hereby incorporated by reference, and include what are commonly known in the art as beta-D-oxy LNA and alpha-L-oxy LNA nucleosides.

In some embodiments, —X—Y— is —S—CH₂—, W is oxygen and R¹, R², R³, R⁵ and R⁵* are all hydrogen at the same time. Such thio LNA nucleosides are disclosed in WO 99/014226 and WO 2004/046160 which are hereby incorporated by reference.

In some embodiments, —X—Y— is —NH—CH₂—, W is oxygen and R¹, R², R³, R⁵ and R⁵* are all hydrogen at the same time. Such amino LNA nucleosides are disclosed in WO 99/014226 and WO 2004/046160, which are hereby incorporated by reference.

In some embodiments, —X—Y— is —O—CH₂CH₂— or —OCH₂CH₂CH₂—, W is oxygen, and R¹, R², R³, R⁵ and R⁵* are all hydrogen at the same time. Such LNA nucleosides are disclosed in WO 00/047599 and Morita et al., Blioorgwdc & Med. Chem. Lett. 12, 73-76, which are hereby incorporated by reference, and include what are commonly known in the art as 2′-O-4′C-ethylene bridged nucleic acids (ENA).

In some embodiments, —X—Y— is —O—CH₂—, W is oxygen, R¹, R², R³ are all hydrogen at the same time, one of R⁵ and R⁵* is hydrogen and the other one is not hydrogen, such as alkyl, for example methyl. Such 5′ substituted LNA nucleosides are disclosed in WO 2007/134181, which is hereby incorporated by reference.

In some embodiments, —X—Y— is —O—CR^{a}R^{b}—, wherein one or both of R^{a} and R^{b} are not hydrogen, in particular alkyl such as methyl, W is oxygen, R¹, R², R³ are all hydrogen at the same time, one of R⁵ and R⁵* is hydrogen and the other one is not hydrogen, in particular alkyl, for example methyl. Such bis modified LNA nucleosides are disclosed in WO 2010/077578, which is hereby incorporated by reference.

In some embodiments, —X—Y— is —O—CH(CH₂—O—CH₃)— (“2′ O-methoxyethyl bicyclic nucleic acid”, Seth et al., J. Org. Chem. 2010, Vol 75(5) pp. 1569-81).

In some embodiments, —X—Y— is —O—CHR^{a}—, W is oxygen and R¹, R², R³, R⁵ and R⁵* are all hydrogen at the same time. Such 6′-substituted LNA nucleosides are disclosed in WO 2010/036698 and WO 2007/090071, which are both hereby incorporated by reference. In such 6′-substituted LNA nucleosides, R^{a} is in particular C1-C6 alkyl, such as methyl.

In some embodiments, —X—Y— is —O—CH(CH₂—O—CH₃)—, W is oxygen and R¹, R², R³, R⁵ and R⁵* are all hydrogen at the same time. Such LNA nucleosides are also known in the art as cyclic MOEs (cMOE) and are disclosed in WO 2007/090071.

In some embodiments, —X—Y— is —O—CH(CH₃)—.

In some embodiments, —X—Y— is —O—CH₂—O—CH₂-(Seth et al., J. Org Chem 2010 op. cit.)

In some embodiments, —X—Y— is —O—CH(CH₃)—, W is oxygen and R¹, R², R³, R⁵ and R⁵* are all hydrogen at the same time. Such 6′-methyl LNA nucleosides are also known in the art as cET nucleosides, and may be either (S)-cET or (R)-cET diastereoisomers, as disclosed in WO 2007/090071 (beta-D) and WO 2010/036698 (alpha-L) which are both hereby incorporated by reference.

In some embodiments, —X—Y— is —O—CR^{a}R^{b}—, wherein neither R^{a} nor R^{b} is hydrogen, W is oxygen, and R¹, R², R³, R⁵ and R⁵* are all hydrogen at the same time. In certain embodiments, R^{a} and R^{b} are both alkyl at the same time, in particular both methyl at the same time. Such 6′-di-substituted LNA nucleosides are disclosed in WO 2009/006478 which is hereby incorporated by reference.

In some embodiments, —X—Y— is —S—CHR^{a}—, W is oxygen, and R¹, R², R³, R⁵ and R⁵* are all hydrogen at the same time. Such 6′-substituted thio LNA nucleosides are disclosed in WO 2011/156202, which is hereby incorporated by reference. In certain embodiments of such 6′-substituted thio LNA, R^{a} is alkyl, in particular methyl.

In some embodiments, —X—Y— is —C═CH₂)C(R^{a}R^{b})—, such as, W is oxygen, and R¹, R², R³, R⁵ and R⁵* are all hydrogen at the same time. Such vinyl carbo LNA nucleosides are disclosed in WO 2008/154401 and WO 2009/067647, which are both hereby incorporated by reference.

In some embodiments, —X—Y— is —N(OR^{a})—CH₂—, W is oxygen and R¹, R², R³, R⁵ and R⁵* are all hydrogen at the same time. In some embodiments. R^{a} is alkyl such as methyl. Such LNA nucleosides are also known as N substituted LNAs and are disclosed in WO 2008/150729, which is hereby incorporated by reference.

In some embodiments, —X—Y— is —O—NCH₃— (Seth et al., J. Org Chem 2010 op. cit.).

In some embodiments, —X—Y— is ON(R^{a})— —N(R^{a})—O—, —NR—CR^{a}R^{b}—CR^{a}R^{b}—, or —NR^{a}—CR^{a}R^{b}—, W is oxygen, and R¹, R², R³, R⁵ and R⁵* are all hydrogen at the same time. In certain embodiments, R^{a} is alkyl, such as methyl. (Seth et al., J. Org. Chem 2010 op. cit.).

In some embodiments, R⁵ and R⁵* are both hydrogen at the same time. In other embodiments, one of R⁵ and R⁵* is hydrogen and the other one is alkyl, such as methyl. In such embodiments, R¹, R² and R³ can be in particular hydrogen and —X—Y— can be in particular —O—CH₂— or —O—CHC(R^{a})₃—, such as —O—CH(CH)—.

In some embodiments, —X—Y— is —CR^{a}R^{b}—O—CR^{a}R^{b}—, such as —CH₂—O—CH₂—, W is oxygen and R¹, R², R³, R⁵ and R⁵* are all hydrogen at the same time. In such embodiments, R^{a} can be in particular alkyl such as methyl. Such LNA nucleosides are also known as conformationally restricted nucleotides (CRNs) and are disclosed in WO 2013/036868, which is hereby incorporated by reference.

In some embodiments, —X—Y— is —O—CR^{a}R^{b}—O—CR^{a}R^{b}—, such as —O—CH₂—O—CH₂—, W is oxygen and R¹, R², R³, R⁵ and R⁵* are all hydrogen at the same time. In certain embodiments, R^{a} can be in particular alkyl such as methyl. Such LNA nucleosides are also known as COC nucleotides and are disclosed in Mitsuoka et al., Nucleic Acids Research 2009, 37(4), 1225-1238, which is hereby incorporated by reference.

It will be recognized than, unless specified, the LNA nucleosides may be in the beta-D or alpha-L stereoisoform.

Certain examples of LNA nucleosides are presented in Scheme 1.

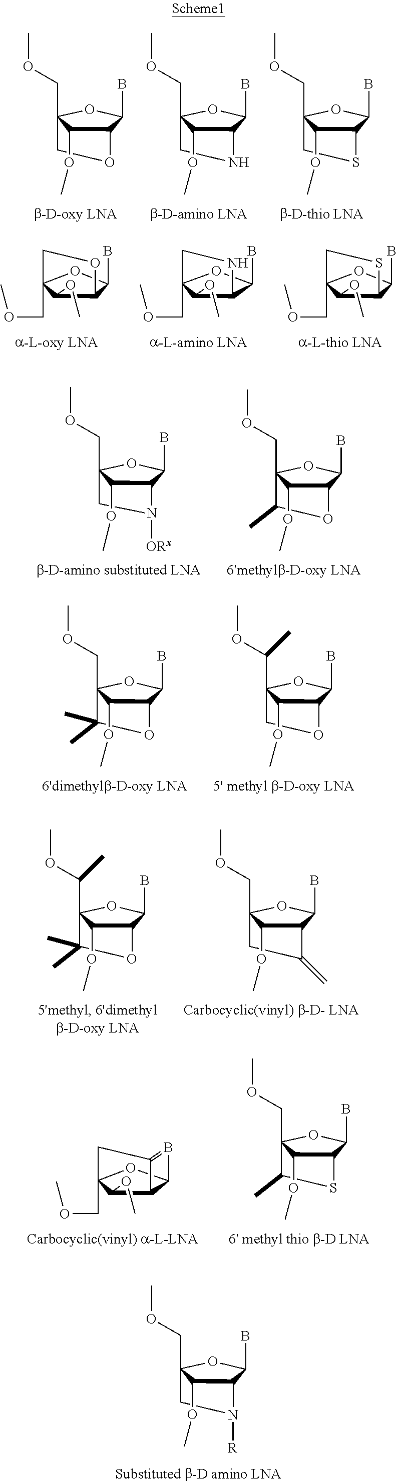

As illustrated elsewhere, in some embodiments of the disclosure the LNA nucleosides in the oligonucleotides are beta-D-oxy-LNA nucleosides.

### II.E. Nuclease Mediated Degradation

Nuclease mediated degradation refers to an oligonucleotide capable of mediating degradation of a complementary nucleotide sequence when forming a duplex with such a sequence.

In some embodiments, the oligonucleotide may function via nuclease mediated degradation of the target nucleic acid, where the oligonucleotides of the disclosure are capable of recruiting a nuclease, particularly and endonuclease, preferably endoribonuclease (RNase), such as RNase H. Examples of oligonucleotide designs which operate via nuclease mediated mechanisms are oligonucleotides which typically comprise a region of at least 5 or 6 DNA nucleosides and are flanked on one side or both sides by affinity enhancing nucleosides, for example gapmers, headmers and tailmers.

II.F. RNase H Activity and Recruitment

The RNase H activity of an antisense oligonucleotide refers to its ability to recruit RNase H when in a duplex with a complementary RNA molecule and induce degradation of the complementary RNA molecule. WO01/23613 provides in vitro methods for determining RNaseH activity, which may be used to determine the ability to recruit RNaseH. Typically, an oligonucleotide is deemed capable of recruiting RNase H if, when provided with a complementary target nucleic acid sequence, it has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers, with phosphorothioate linkages between all monomers in the oligonucleotide, and using the methodology provided by Example 91-95 of WO01/23613.

In some embodiments, an oligonucleotide is deemed essentially incapable of recruiting RNaseH if, when provided with the complementary target nucleic acid, the RNaseH initial rate, as measured in pmol/l/min, is less than 20%, such as less than 10%, such as less than 5% of the initial rate determined when using a oligonucleotide having the same base sequence as the oligonucleotide being tested, but containing only DNA monomers, with no 2′ substitutions, with phosphorothioate linkages between all monomers in the oligonucleotide, and using the methodology provided by Example 91-95 of WO01/23613.

II.G. ASO Design

The ASO of the disclosure can comprise a nucleotide sequence which comprises both nucleosides and nucleoside analogs, and can be in the form of a gapmer, blockmer, mixmer, headmer, tailmer, or totalmer. Examples of configurations of a gapmer, blockmer, mixmer, headmer, tailmer, or totalmer that can be used with the ASO of the disclosure are described in U.S. Patent Appl. Publ. No. 2012/0322851.

The term “gapmer” as used herein refers to an antisense oligonucleotide which comprises a region of RNase H recruiting oligonucleotides (gap) which is flanked 5′ and 3′ by one or more affinity enhancing modified nucleosides (flanks). The terms “headmers” and “tailmers” are oligonucleotides capable of recruiting RNase H where one of the flanks is missing, i.e., only one of the ends of the oligonucleotide comprises affinity enhancing modified nucleosides. For headmers, the 3′ flank is missing (i.e., the 5′ flank comprise affinity enhancing modified nucleosides) and for tailmers, the 5′ flank is missing (i.e., the 3′ flank comprises affinity enhancing modified nucleosides). The term “LNA gapmer” is a gapmer oligonucleotide wherein at least one of the affinity enhancing modified nucleosides is an LNA nucleoside. The term “mixed wing gapmer” refers to an LNA gapmer wherein the flank regions comprise at least one LNA nucleoside and at least one DNA nucleoside or non-LNA modified nucleoside, such as at least one 2′ substituted modified nucleoside, such as, for example, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-Fluro-DNA, arabino nucleic acid (ANA), and 2′-Fluoro-ANA nucleoside(s).

Other “chimeric” ASOs, called “mixmers”, consist of an alternating composition of (i) DNA monomers or nucleoside analog monomers recognizable and cleavable by RNase, and (ii) non-RNase recruiting nucleoside analog monomers.

A “totalmer” is a single stranded ASO which only comprises non-naturally occurring nucleotides or nucleotide analogs.

In some embodiments, in addition to enhancing affinity of the ASO for the target region, some nucleoside analogs also mediate RNase (e.g., RNaseH) binding and cleavage. Since α-L-LNA monomers recruit RNaseH activity to a certain extent, in some embodiments, gap regions (e.g., region B as referred to herein) of ASOs containing α-L-LNA monomers consist of fewer monomers recognizable and cleavable by the RNaseH, and more flexibility in the mixmer construction is introduced.

II.G.1. Gapmer Design

In some embodiments, the ASO of the disclosure is a gapmer and comprises a contiguous stretch of nucleotides (e.g., one or more DNA) which is capable of recruiting an RNase, such as RNaseH, referred to herein in as region B (B), wherein region B is flanked at both 5′ and 3′ by regions of nucleoside analogs 5′ and 3′ to the contiguous stretch of nucleotides of region B— these regions are referred to as regions A (A) and C (C), respectively. In some embodiments, the nucleoside analogs are sugar modified nucleosides (e.g., high affinity sugar modified nucleosides). In certain embodiments, the sugar modified nucleosides of regions A and C enhance the affinity of the ASO for the target nucleic acid (i.e., affinity enhancing 2′ sugar modified nucleosides). In some embodiments, the sugar modified nucleosides are 2′ sugar modified nucleosides, such as high affinity 2′ sugar modifications, such as LNA or 2′-MOE.

In a gapmer, the 5′ and 3′ most nucleosides of region B are DNA nucleosides, and are positioned adjacent to nucleoside analogs (e.g., high affinity sugar modified nucleosides) of regions A and C, respectively. In some embodiments, regions A and C can be further defined by having nucleoside analogs at the end most distant from region B (i.e., at the 5′ end of region A and at the 3′ end of region C).

In some embodiments, the ASOs of the present disclosure comprise a nucleotide sequence of formula (5′ to 3′) A-B-C, wherein: (A) (5′ region or a first wing sequence) comprises at least one nucleoside analog (e.g., 3-5 LNA units); (B) comprises at least four consecutive nucleosides (e.g., 4-24 DNA units), which are capable of recruiting RNase (when formed in a duplex with a complementary RNA molecule, such as the pre-mRNA or mRNA target); and (C) (3′ region or a second wing sequence) comprises at least one nucleoside analog (e.g., 3-5 LNA units).

In some embodiments, region A comprises 3-5 nucleotide analogs, such as LNA, region B consists of 6-24 (e.g., 6, 7, 8, 9, 10, 11, 12, 13, or 14) DNA units, and region C consists of 3 or 4 nucleotide analogs, such as LNA. Such designs include (A-B-C) 3-14-3, 3-11-3, 3-12-3, 3-13-3, 4-9-4, 4-10-4, 4-11-4, 4-12-4, and 5-10-5. In some embodiments, the ASO has a design of LLLDₙLLL, LLLLDₙILLLL, or LLILLDₙLLLL, wherein the L is a nucleoside analog, the D is DNA, and n can be any integer between 4 and 24. In some embodiments, n can be any integer between 6 and 14. In some embodiments, n can be any integer between 8 and 12.

Further gapmer designs are disclosed in WO2004/046160, WO 2007/146511, and WO2008/113832, each of which is hereby incorporated by reference in its entirety.

II.H. Internucleotide Linkages

The monomers of the ASOs described herein are coupled together via linkage groups. Suitably, each monomer is linked to the 3′ adjacent monomer via a linkage group.

The person having ordinary skill in the art would understand that, in the context of the present disclosure, the 5′ monomer at the end of an ASO does not comprise a 5′ linkage group, although it may or may not comprise a 5′ terminal group.

The terms “linkage group” or “internucleoside linkage” are intended to mean a group capable of covalently coupling together two nucleosides. Specific and preferred examples include phosphate groups and phosphorothioate groups.

The nucleosides of the ASO of the disclosure or contiguous nucleosides sequence thereof are coupled together via linkage groups. Suitably each nucleoside is linked to the 3′ adjacent nucleoside via a linkage group.

In some embodiments, the internucleoside linkage is modified from its normal phosphodiester to one that is more resistant to nuclease attack, such as phosphorothioate, which is cleavable by RNaseH, also allows that route of antisense inhibition in reducing the expression of the target gene. In some embodiments, at least 75%, at least 80%, at least 85%, at least 90%, at least 91%, at least 92%, at least 93%, at least 94%, at least 95%, at least 96%, at least 97%, at least 98%, at least 99% a, or 100% of internucleoside linkages are modified.

II.I. Conjugates

The term conjugate as used herein refers to an ASO which is covalently linked to a non-nucleotide moiety (conjugate moiety or region C or third region).

Conjugation of the ASO of the disclosure to one or more non-nucleotide moieties may improve the pharmacology of the ASO, e.g., by affecting the activity, cellular distribution, cellular uptake, or stability of the ASO. In some embodiments, the non-nucleotide moieties modify or enhance the pharmacokinetic properties of the ASO by improving cellular distribution, bioavailability, metabolism, excretion, permeability, and/or cellular uptake of the ASO. In certain embodiments, the non-nucleotide moieties may target the ASO to a specific organ, tissue, or cell type and thereby enhance the effectiveness of the ASO in that organ, tissue, or cell type. In other embodiments, the non-nucleotide moieties reduce the activity of the ASO in non-target cell types, tissues, or organs, e.g., off target activity or activity in non-target cell types, tissues, or organs. WO 93/07883 and WO2013/033230 provides suitable conjugate moieties. Further suitable conjugate moieties are those capable of binding to the asialoglycoprotein receptor (ASGPr). In particular, tri-valent N-acetylgalactosamine conjugate moieties are suitable for binding to the ASGPr, see, e.g., WO 2014/076196, WO 2014/207232, and WO 2014/179620, each of which are hereby incorporated by reference.

In some embodiments, the non-nucleotide moiety (conjugate moiety) is selected from the group consisting of carbohydrates, cell surface receptor ligands, drug substances, hormones, lipophilic substances, polymers, proteins, peptides, toxins (e.g., bacterial toxins), vitamins, viral proteins (e.g., capsids), and combinations thereof.

II.J. Activated ASOs

The term “activated ASO,” as used herein, refers to an ASO that is covalently linked (i.e., functionalized) to at least one functional moiety that permits covalent linkage of the ASO to one or more conjugated moieties, i.e., moieties that are not themselves nucleic acids or monomers, to form the conjugates herein described. Typically, a functional moiety will comprise a chemical group that is capable of covalently bonding to the ASO via, e.g., a 3′-hydroxyl group or the exocyclic NH₂ group of the adenine base, a spacer that can be hydrophilic and a terminal group that is capable of binding to a conjugated moiety (e.g., an amino, sulfhydryl or hydroxyl group). In some embodiments, this terminal group is not protected, e.g., is an NH₂ group. In other embodiments, the terminal group is protected, for example, by any suitable protecting group such as those described in “Protective Groups in Organic Synthesis” by Theodora W Greene and Peter G M Wuts, 3rd edition (John Wiley & Sons, 1999), which is hereby incorporated by reference.

In some embodiments, ASOs of the disclosure are functionalized at the 5′ end in order to allow covalent attachment of the conjugated moiety to the 5′ end of the ASO. In other embodiments. ASOs of the disclosure can be functionalized at the 3′ end. In still other embodiments, ASOs of the disclosure can be functionalized along the backbone or on the heterocyclic base moiety. In yet other embodiments, ASOs of the disclosure can be functionalized at more than one position independently selected from the 5′ end, the 3′ end, the backbone and the base.

In some embodiments, activated ASOs of the disclosure are synthesized by incorporating during the synthesis one or more monomers that is covalently attached to a functional moiety. In other embodiments, activated ASOs of the disclosure are synthesized with monomers that have not been functionalized, and the ASO is functionalized upon completion of synthesis.

### III. Pharmaceutical Compositions and Administration Routes

The ASO of the disclosure can be used in pharmaceutical formulations and compositions. In some embodiments, such compositions comprise a pharmaceutically acceptable diluent, carrier, salt, or adjuvant. In certain embodiments, a pharmaceutically acceptable salt comprises a sodium salt, a potassium salt, or an ammonium salt.

The ASO of the disclosure can be included in a unit formulation such as in a pharmaceutically acceptable carrier or diluent in an amount sufficient to deliver to a patient a therapeutically effective amount without causing serious side effects in the treated patient. However, in some forms of therapy, serious side effects may be acceptable in terms of ensuring a positive outcome to the therapeutic treatment.

The formulated drug may comprise pharmaceutically acceptable binding agents and adjuvants. Capsules, tablets, or pills can contain for example the following compounds: microcrystalline cellulose, gum or gelatin as binders; starch or lactose as excipients; stearates as lubricants; various sweetening or flavoring agents. For capsules, the dosage unit can contain a liquid carrier like fatty oils. Likewise, coatings of sugar or enteric agents can be part of the dosage unit. The ASO formulations can also be emulsions of the active pharmaceutical ingredients and a lipid forming a micellular emulsion.

The pharmaceutical compositions of the present disclosure can be administered in a number of ways depending upon whether local or systemic treatment is desired and upon the area to be treated. Administration can be (a) oral; (b) pulmonary, e.g., by inhalation or insufflation of powders or aerosols, including by nebulizer; intratracheal, intranasal, (c) topical including epidermal, transdermal, ophthalmic and to mucous membranes including vaginal and rectal delivery; or (d) parenteral including intravenous, intraarterial, subcutaneous, intraperitoneal or intramuscular injection or infusion; or intracranial, e.g., intrathecal, intra-cerebroventricular, or intraventricular, administration. In some embodiments, the ASO is administered intravenously, intraperitoneally, orally, topically, or as a bolus injection or administered directly in to the target organ. In some embodiments, the ASO is administered intracardially or intraventricularly as a bolus injection. In some embodiments, the ASO is administered subcutaneously. In some embodiments, the ASO is administered orally.

Pharmaceutical compositions and formulations for topical administration can include transdermal patches, ointments, lotions, creams, gels, drops, sprays, suppositories, liquids and powders. Conventional pharmaceutical carriers, aqueous, powder or oily bases, thickeners and the like may be necessary or desirable. Examples of topical formulations include those in which the ASO of the disclosure are in admixture with a topical delivery agent such as lipids, liposomes, fatty acids, fatty acid esters, steroids, chelating agents and surfactants. Compositions and formulations for oral administration include but are not limited to powders or granules, microparticulates, nanoparticulates, suspensions or solutions in water or non-aqueous media, capsules, gel capsules, sachets, tablets or minitablets. Compositions and formulations for parenteral, intrathecal, intra-cerebroventricular, or intraventricular administration can include sterile aqueous solutions which can also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome-containing formulations. These compositions may be generated from a variety of components that include, but are not limited to, preformed liquids, self-emulsifying solids and self-emulsifying semisolids. Delivery of drug to the target tissue can be enhanced by carrier-mediated delivery including, but not limited to, cationic liposomes, cyclodextrins, porphyrin derivatives, branched chain dendrimers, polyethylenimine polymers, nanoparticles and microspheres (Dass C R. J Pharm Pharmacol 2002; 54(1):3-27).

The pharmaceutical formulations of the present disclosure, which can conveniently be presented in unit dosage form, can be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

For parenteral, subcutaneous, intradermal, or topical administration the formulation can include a sterile diluent, buffers, regulators of tonicity and antibacterials. The active ASOs can be prepared with carriers that protect against degradation or immediate elimination from the body, including implants or microcapsules with controlled release properties. For intravenous administration the carriers can be physiological saline or phosphate buffered saline. International Publication No. WO2007/031091 (A2), published Mar. 22, 2007, further provides suitable pharmaceutically acceptable diluent, carrier and adjuvants—which are hereby incorporated by reference.

### IV. Diagnostics

This disclosure further provides a diagnostic method useful during diagnosis of cardiovascular diseases, e.g., a heart failure. Non-limiting examples of cardiovascular diseases that can be diagnosed with the present ASOs include, but are not limited to, coronary artery disease, stroke, heart failure, hypertensive heart disease, rheumatic heart disease, cardiomyopathy, heart arrhythmia, congenital heart disease, valvular heart disease carditis, aortic aneurysms, peripheral artery disease, thromboembolic disease, and venous thrombosis. In some embodiments, heart failure comprises a left-sided heart failure, a right-sided heart failure, a congestive heart failure, a heart failure with reduced ejection fraction (HFrEF), a heart failure with preserved ejection fraction (HFpEF), a heart failure with mid-range ejection fraction (HFmrEF), a hypertrophic cardiomyopathy (HCM), a hypertensive heart disease (HHD), or hypertensive hypertrophic cardiomyopathy.

The ASOs of the disclosure can be used to measure expression of CAMK2D transcript in a tissue or body fluid from an individual and comparing the measured expression level with a standard CAMK2D transcript expression level in normal tissue or body fluid, whereby an increase in the expression level compared to the standard is indicative of a disorder treatable by an ASO of the disclosure.

The ASOs of the disclosure can be used to assay CAMK2D transcript levels in a biological sample using any methods known to those of skill in the art. (Touboul et. al., Anticancer Res. (2002) 22 (6A): 3349-56; Verjout et. al., Mutal. Res. (2000) 640: 127-38); Stowe et. al., J. Virol. Methods (1998) 75 (1): 93-91).

The term “biological sample” refers to any biological sample obtained from an individual, cell line, tissue culture, or other source of cells potentially expressing CAMK2D transcript. Methods for obtaining such a biological sample from mammals are well known in the art.

### V. Kits Comprising ASOs

This disclosure further provides kits that comprise an ASO of the disclosure described herein and that can be used to perform the methods described herein. In certain embodiments, a kit comprises at least one ASO in one or more containers. In some embodiments, the kits contain all of the components necessary and/or sufficient to perform a detection assay, including all controls, directions for performing assays, and any necessary software for analysis and presentation of results. One skilled in the art will readily recognize that the disclosed ASO can be readily incorporated into one of the established kit formats which are well known in the art.

### VI. Methods of Using

The ASOs of the disclosure can be utilized as research reagents for, for example, diagnostics, therapeutics, and prophylaxis.

In research, such ASOs can be used to specifically inhibit the synthesis of CAMK2D protein (typically by degrading or inhibiting the mRNA and thereby prevent protein formation) in cells and experimental animals thereby facilitating functional analysis of the target or an appraisal of its usefulness as a target for therapeutic intervention. Further provided are methods of down-regulating the expression of CAMK2D mRNA and/or CAMK2D protein in cells or tissues comprising contacting the cells or tissues, in vitro or in viva, with an effective amount of one or more of the ASOs, conjugates or compositions of the disclosure.

In diagnostics, the ASOs can be used to detect and quantitate CAMK2D transcript expression in cell and tissues by northern blotting, in-situ hybridization, or similar techniques.

For therapeutics, an animal or a human, suspected of having a disease or disorder, which can be treated by modulating the expression of CAMK2D transcript and/or CAMK2D protein is treated by administering ASOs in accordance with this disclosure. Further provided are methods of treating a mammal, such as treating a human, suspected of having or being prone to a disease or condition, associated with increased expression of CAMK2D transcript and/or CAMK2D protein by administering a therapeutically or prophylactically effective amount of one or more of the ASOs or compositions of the disclosure. The ASO, a conjugate, or a pharmaceutical composition according to the disclosure is typically administered in an effective amount. In some embodiments, the ASO or conjugate of the disclosure is used in therapy.

The disclosure further provides for an ASO according to the disclosure, for use for the treatment of one or more of the cardiovascular diseases referred to herein, such as a disease selected from a coronary artery disease, stroke, heart failure, hypertensive heart disease, rheumatic heart disease, cardiomyopathy, heart arrhythmia, congenital heart disease, valvular heart disease carditis, aortic aneurysms, peripheral artery disease, thromboembolic disease, and venous thrombosis.

In certain embodiments, the disease, disorder, or condition is associated with overexpression of CAMK2D gene transcript and/or CAMK2D protein.

The disclosure also provides for methods of inhibiting (e.g., by reducing) the expression of CAMK2D gene transcript and/or CAMK2D protein in a cell or a tissue, the method comprising contacting the cell or tissue, in vitro or in vivo, with an effective amount of one or more ASOs, conjugates, or pharmaceutical compositions thereof, of the disclosure to affect degradation of expression of CAMK2D gene transcript thereby reducing CAMK2D protein.

The disclosure also provides for the use of the ASO or conjugate of the disclosure as described for the manufacture of a medicament for the treatment of a disorder as referred to herein, or for a method of the treatment of as a disorder as referred to herein.

The disclosure further provides for a method for inhibiting or reducing CAMK2D protein in a cell which is expressing CAMK2D comprising administering an ASO or a conjugate according to the disclosure to the cell so as to affect the inhibition or reduction of CAMK2D protein in the cell.

The disclosure includes a method of reducing, ameliorating, preventing, or treating hyperexcitability of motor neurons (e.g., such as those found in cardiomyocytes) in a subject in need thereof comprising administering an ASO or a conjugate according to the disclosure.

The disclosure also provides for a method for treating a disorder as referred to herein the method comprising administering an ASO or a conjugate according to the disclosure as herein described and/or a pharmaceutical composition according to the disclosure to a patient in need thereof.

The ASOs and other compositions according to the disclosure can be used for the treatment of conditions associated with over expression of CAMK2D protein.

Generally stated, one aspect of the disclosure is directed to a method of treating a mammal suffering from or susceptible to conditions associated with abnormal levels of CAMK2D, comprising administering to the mammal and therapeutically effective amount of an ASO targeted to CAMK2D transcript that comprises one or more LNA units. The ASO, a conjugate, or a pharmaceutical composition according to the disclosure is typically administered in an effective amount.

An interesting aspect of the disclosure is directed to the use of an ASO (compound) as defined herein or a conjugate as defined herein for the preparation of a medicament for the treatment of a disease, disorder or condition as referred to herein.

The methods of the disclosure can be employed for treatment or prophylaxis against diseases caused by abnormal levels of CAMK2D protein. In some embodiments, diseases caused by abnormal levels of CAMK2D protein are cardiovascular diseases. In certain embodiments, cardiovascular diseases can include a coronary artery disease, stroke, heart failure, hypertensive heart disease, rheumatic heart disease, cardiomyopathy, heart arrhythmia, congenital heart disease, valvular heart disease carditis, aortic aneurysms, peripheral artery disease, thromboembolic disease, and venous thrombosis.

In certain embodiments, the cardiovascular disease is a heart failure, which can include a left-sided heart failure, a right-sided heart failure, congestive heart failure, a heart failure with reduced ejection fraction (HFrEF), a heart failure with preserved ejection fraction (HFpEF), a heart failure with mid-range ejection fraction (HFmrEF), a hypertrophic cardiomyopathy (HCM), a hypertensive heart disease (HHD), or hypertensive hypertrophic cardiomyopathy.

Alternatively stated, in some embodiments, the disclosure is furthermore directed to a method for treating abnormal levels of CAMK2D protein, the method comprising administering a ASO of the disclosure, or a conjugate of the disclosure or a pharmaceutical composition of the disclosure to a patient in need thereof.

The disclosure also relates to an ASO, a composition or a conjugate as defined herein for use as a medicament.

The disclosure further relates to use of a compound, composition, or a conjugate as defined herein for the manufacture of a medicament for the treatment of abnormal levels of CAMK2D protein or expression of mutant forms of CAMK2D protein (such as allelic variants, wherein the allelic variants are associated with one of the diseases referred to herein).

A patient who is in need of treatment is a patient suffering from or likely to suffer from the disease or disorder.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis el al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooke, Antisense drug Technology: Principles, Strategies and Applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1: Construction of ASOs

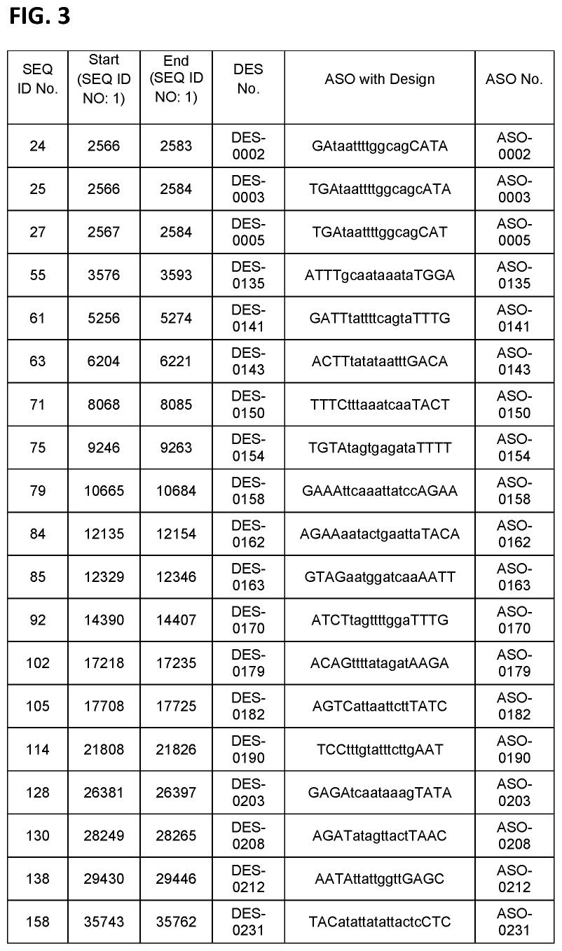

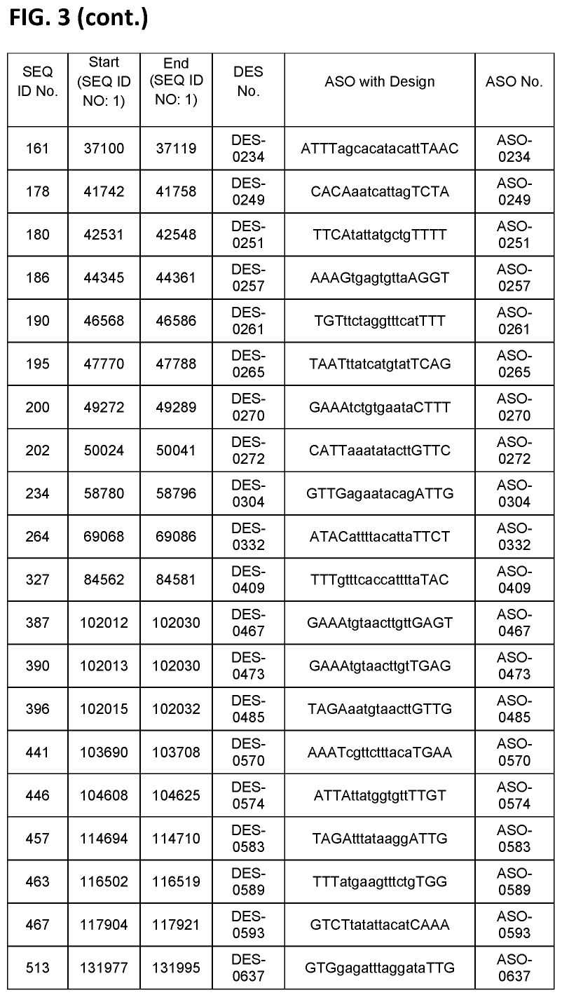

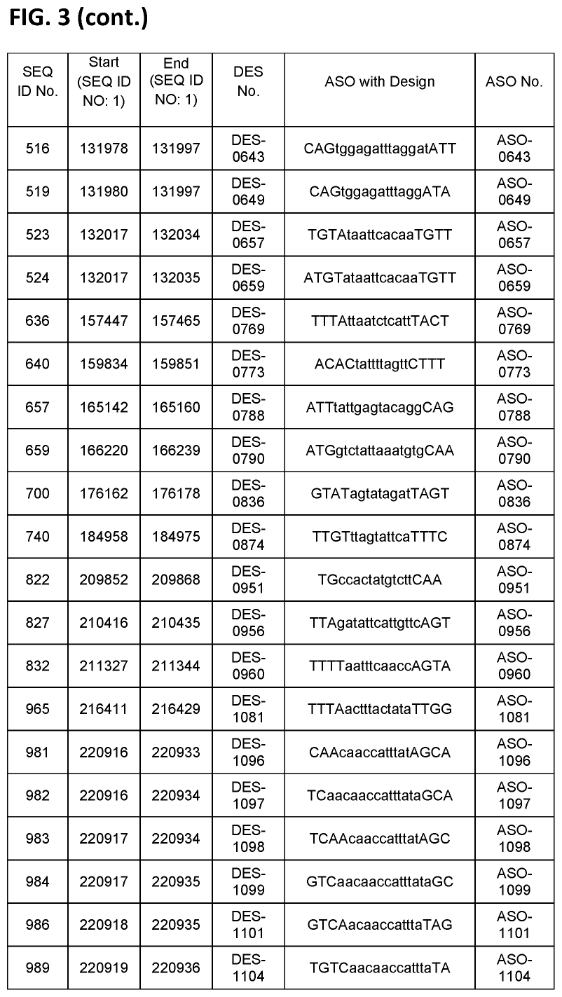

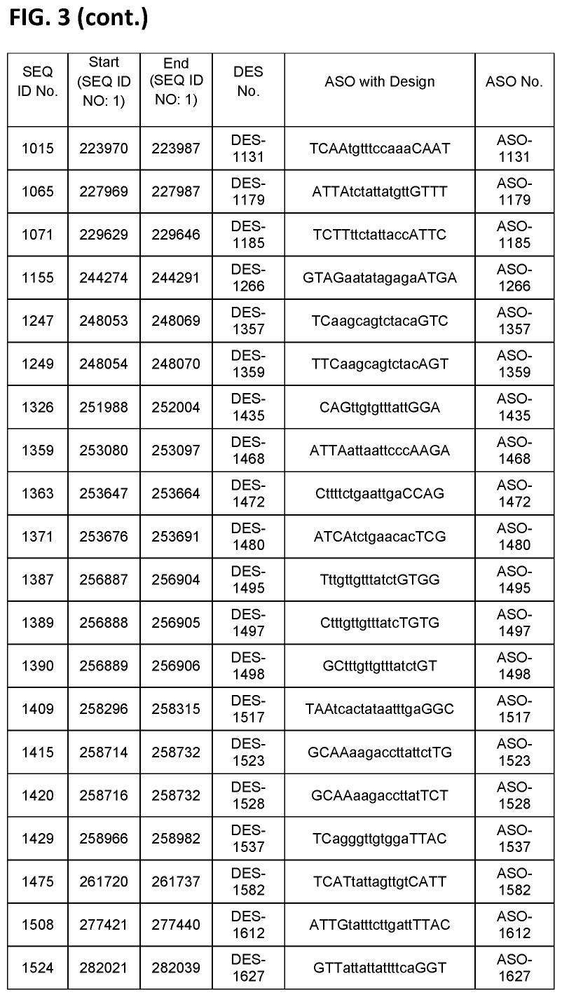

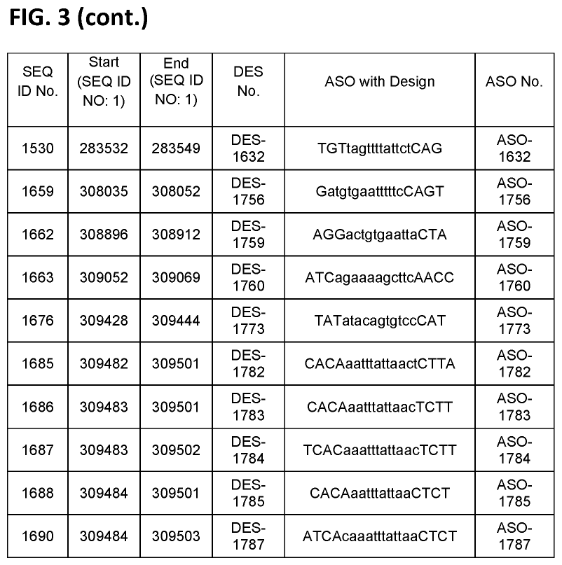

Antisense oligonucleotides described herein were designed to target various regions in the CAMK2D pre-mRNA (SEQ ID NO: 1). For example, the ASOs were constructed to target the regions denoted using the start and end sites of SEQ ID NO: 1, as shown in FIGS. 1A and 1B. The exemplary sequences of the ASOs of the present disclosure are provided in FIGS. 1A and 113. In some embodiments, the ASOs were designed to be gapmers as shown in FIG. 3. The disclosed gapmers were constructed to contain locked nucleic acids—LNAs (upper case letters). For example, a gapmer can have beta-deoxy LNA at the 5′ end and the 3′ end and have a phosphorothioate backbone. But the LNA can also be substituted with any other nucleoside analogs and the backbone can be other types of backbones (e.g., phosphodiester linkage, a phosphotriester linkage, a methylphosphonate linkage, a phosphoroamidate linkage, or any combinations thereof).

The ASOs were synthesized using methods well known in the art. Exemplary methods of preparing such ASOs are described in Barciszewski et al., Chapter 10—“Locked Nucleic Acid Aptamers” in Nucleic Acid and Peptide Aptamers: Methods aid Protocols, vol. 535, Gunter Mayer (ed.)(2009), the entire contents of which is hereby expressly incorporated by reference herein.

### Example 2: qPCR Assay to Measure Reduction of CAMK2D mRNA in HEK293 Cells

The ASOs of the present disclosure were tested for their ability to reduce CAMK2D mRNA expression in human embryonic kidney cells (HEK293) (European Collection of Authenticated Cell Cultures (ECACC), catalog no. 85120602). The HEK293 cells were grown in cell culture media (DMEM AQ D0819, 10% FBS, and Pen/Strep). Every 5 days, cells were trypsinized by washing with Phosphate Buffered Saline (PBS), followed by addition of 0.25% Trypsin-EDTA solution, 2-3 minutes incubation at 37° C., and trituration before cell seeding. Cells were maintained in culture for up to 15 passages.

For experimental use, 3,500 cells per well were seeded in 96 well plates in 100 μL growth media. ASOs were prepared from a 750 μM stock and dissolved in PBS. Approximately 24 hours after seeding the cells, ASOs were added to the cells at a final concentration of 25 μM. Cells were then incubated for 3 days without any media change. After incubation, cells were harvested by removal of media followed by addition of 125 μL PURELINK®Pro 96 Lysis buffer and 125 μL 70% ethanol. Then, RNA was purified according to the manufacture's instruction and eluted in a final volume of 50 μL water, resulting in an RNA concentration of 10-20 ng/μL. Next, RNA was diluted 10 fold in water prior to the one-step qPCR reaction.

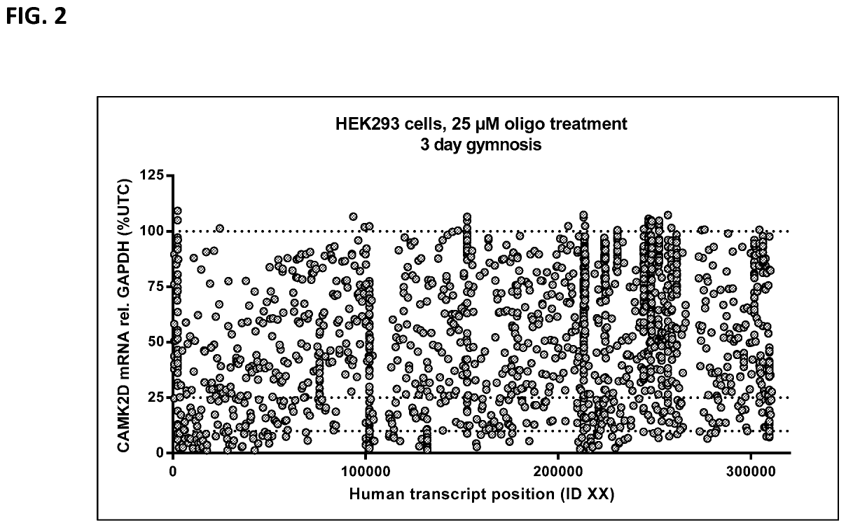

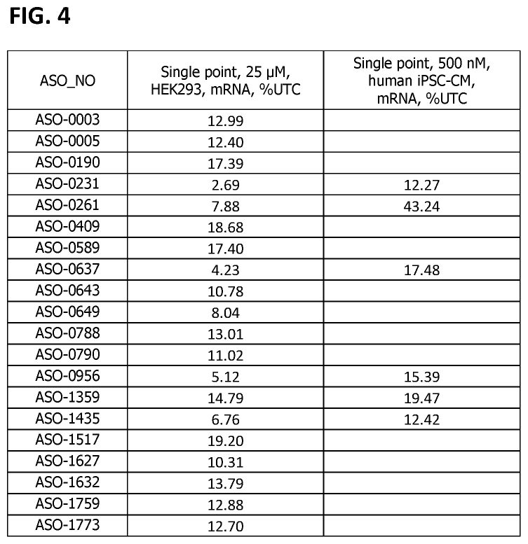

For the one-step qPCR reaction, qPCR-mix (qScriptTMXLE 1-step RT-qPCR TOUGHMIX®Low ROX from QauntaBio) was mixed with two Taqman probes at a ratio 10:1:1 (qPCR mix: probe1:probe2) to generate the mastermix. Taqman probes were acquired from LifeTechnologies: CAMK2D_Hs009943538_m1; GAPDH 4325792. The mastermix (6 μL) and RNA (4 μL, 1-2 ng/μL) were then mixed in a qPCR plate (MICROAMP® optical 384 well, catalog no. 4309849). After sealing the plate, the plate was given a quick spin, 1000 g for 1 minute at RT, and transferred to a Viia™ 7 system (Applied Biosystems, Thermo). The following PCR conditions were used: 50° C. for 15 minutes; 95° C. for 3 minutes; 40 cycles of: 95° C. for 5 sec, followed by a temperature decrease of 1.6° C./sec, followed by 60° C. for 45 sec. The data was analyzed using the QUANTSTUDIO™ Real_time PCR Software. The percent inhibition for the ASO treated samples was calculated relative to the control treated samples. Results are shown in FIGS. 2 and 4.

### Example 3: QUANTIGENE® Analysis (96-Well Assay) to Measure CAMK2D mRNA Reduction in Human Inducible Pluripotent Stem Cell-Derived Cardiomyocytes (hiPSC-CM)

The ability of ASOs to reduce human CAMK2D mRNA was measured in vitro by QUANTIGENE® analysis. Human inducible pluripotent stem cell-derived cardiomyocytes (hiPSC-CMs) from Cellular Dynamics International (“iCell²”) cells were thawed, plated, and cultured per the manufacturer's instructions. These cardiomyocytes are derived from human induced pluripotent stem cells, which were first successfully differentiated into functional cardiomyocytes back in 2009. Zhang et al., Circ Res 104(4):230-41 (2009). Since then, hiPSC-CMs have been used to study various aspects of the human heart and related diseases. Because these cells bear the genetic traits of the human donors from whom they are obtained, they are often to be better predictors of human physiology or pathophysiology compared to existing animal models. Blazeski et al., Pog Biophys Mol Biol 110:166-177 (2012).

Workflow: Prior to cell seeding, pre-collagen-coated 96-well plates were coated with fibronectin as follows. Fibronectin (1 mg/mL) was diluted 1:100 in PBS (—Ca²⁺, —Mg²⁺) and 50 μL of dilute fibronectin solution was added to each well of the 96-well plate. The plate was gently shaken horizontally to ensure an even coating of fibronectin on the bottom of each well. Then the plates were incubated at 37° C. for 90 minutes. Cells were added to the plates immediately following aspiration of the fibronectin solution as per the manufacturer's instructions. Cells were seeded at 30,000 cells/well in 100 μL of the manufacturer's Plating Media and then incubated at 37° C. and 5% CO₂ for 4 hours. Then the Plating Media was aspirated and replaced with 100 μL of the manufacturer's Maintenance Media. Cells were incubated at 37° C. and 5% CO₂ with media exchange every other day. The ASOs were diluted in water and added to cells at DIV08 (i.e., 8 days post plating). The cells were then incubated at 37° C. and 5% CO₂ for 3 days following ASO addition to achieve steady state reduction of mRNA.

After the incubation, the media was removed and cells were lysed as follows. Working cell lysis buffer was made by adding 1 part proteinase K to 99 parts of QUANTIGENE® 3× lysis buffer and then diluting 1:3 in dH2O. The working lysis buffer was added to the plates at 220 uL/well. After adding lysis buffer, the plate was shaken on a plate shaker for 10 minutes are medium speed (i.e., speed 5-6 out of 10). The plates were then incubated at 55° C. for 30 minute. Following this incubation, the lysates were either frozen at −80° C. or assayed immediately. Measurement of lysate mRNA was performed using the QUANTIGENE® 2.0 Reagent System (AFFYMETRIX®), which quantifies RNA using a branched DNA signal amplification method reliant on the specifically-designed target RNA capture probe set.

Assay: Each well of the capture plate (96-well polystyrene plate coated with capture probes) was loaded with 20 uL of working probe set. Working probe set reagents were generated by combining nuclease-free water (12.05 μL), lysis mixture (6.65 μL), blocking reagent (1 μL), and specific 2.0 probe set (0.3 μL) (human CAMK2D catalogue #SA-3000428 or human POLR2A catalogue #SA-10004) per manufacturer's instructions (QUANTIGENE® 2.0 AFFYMETRLX®). The cell lysates (or 1× lysis buffer for use in background control blank wells) were then added to the capture plates at a volume of 80 μL/well, giving 100 uL of total fluid per well. The plates were sealed using the QUANTIGENE® foil seal in combination with a hand crank sealer. Plates were centrifuged at 240 g for 60 seconds and then incubated for 16-20 hours at 55° C. to hybridize (target RNA capture).

Signal amplification and detection of target RNA began by washing plates with wash buffer 3 times (200, 300, and 300 μL/well in series, with buffer removal between each step) to remove any unbound material, followed by an upside-down centrifugation step for 1 min at 240 g to dry the wells. Next, the 2.0 Pre-Amplifier hybridization reagent (100 μL/well) was added, incubated at 55° C. for 1 hour, then aspirated, and wash buffer was added and aspirated 3 times (200, 300, and 300 uL/well in series, with buffer removal between each step), followed by an upside-down centrifugation step for 1 min at 240 g to dry the wells. The 2.0 Amplifier hybridization reagent was then added (100 μL/well), incubated for 1 hour at 55° C., and then the wash, aspiration, and drying steps were repeated as described above. The 2.0 Label Probe hybridization reagent was added next (100 μL/well), incubated for 1 hour at 50° C., and then the wash, aspiration, and drying steps were repeated as described previously. Then the 2.0 Substrate was added (100 μL/well) to the plates. Plates were incubated for 5 minutes at room temperature and then imaged on a PerkinElmer Envision multilabel plate reader in luminometer mode within 15 minutes.

Data determination: For the gene of interest, the average assay background signal was subtracted from the average signal of each technical replicate. The background-subtracted, average signals for the gene of interest were then normalized to the background-subtracted average signal for the housekeeping POLR2A mRNA. The percent inhibition for the treated sample was calculated relative to the control treated sample lysate. Results of QUANTIGENE® assays for cells treated with the ASOs at a concentration of 500 nM are provided in FIG. 4.

### Example 4: Analysis of CAMK2D mRNA Reduction In Vivo

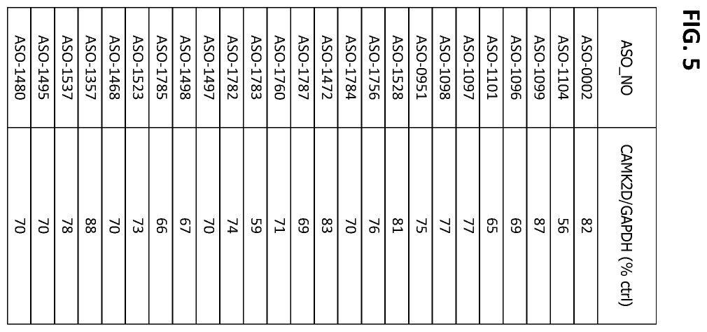

To evaluate the potency of the ASOs in reducing CAMK2D mRNA level in vivo, female C57BL/6JBom mice were subcutaneously administered with one of the ASOs shown in FIG. 5. The ASOs were administered at a dose of 30 mg/kg/day for three consecutive days (day 1, 2, and 3). The mice were observed with regards to behavioral and body weight changes. Mice were sacrificed on day 8 and cardiac tissue was harvested for RNA isolation and analysis as described below.

MagNA Pure tissue lysis buffer (Roche) was added to the cardiac tissue section and homogenized using stainless steel beads until a uniform lysate was obtained. Incubation for 30 minutes at room temperature completed lysis. RNA was isolated using the MagNA Pure96 (Roche) with the Cellular RNA Large Volume Kit.

The RNA concentration was normalized to 5 ng/μl and one-step qPCR was performed using 20 ng RNA, qPCR Taqman Mastermix, and the following Taqman probes: CAMK2D (Thermo Mm00499266_m1) and GAPDH (Thermo 4352339E).

PCR conditions were as follows: 50° C. for 15 minutes; 95° C. for 3 minutes; 40 cycles of: 95° C. for 5 sec. The data was analyzed using the QUANTSTUD™ Real-time PCR Software. The percent inhibition for the ASO treated samples was calculated relative to saline treated samples.

As shown in FIG. 5, all the ASOs tested were able to decrease CAMK2D mRNA level when administered to the C57BL/6JBom mice. Collectively, the results provided herein demonstrate the potency of the ASOs both in vitro and in vivo, and support that CAMK2D-specific ASOs are disease-modifying therapeutics for the treatment of various medical disorders, such as cardiovascular-related diseases or disorders.

1. An antisense oligonucleotide (ASO) comprising a contiguous nucleotide sequence of 10 to 30 nucleotides in length that is at least about 80% complementary to a nucleic acid sequence within a calcium/calmodulin-dependent protein kinase type II delta (CAMK2D) transcript.

2. (canceled)

3. The ASO of claim 1, wherein the CAMK2D transcript is selected from the group consisting of SEQ ID NO: 1 and SEQ ID NO: 2.

4. The ASO of claim 1, wherein the ASO is capable of reducing CAMK2D protein and/or CAMK2D transcript (e.g., mRNA) expression in a human cell which is expressing the CAMK2D protein and/or CAMK2D transcript.

5-7. (canceled)

8. The ASO of claim 1, wherein the ASO is a gapmer.

9-11. (canceled)

12. The ASO of claim 1, comprising a nucleoside analog, wherein one or more of the nucleoside analog is a sugar modified nucleoside.

13. The ASO of claim 12, wherein the nucleoside analog comprises a 2′-O-alkyl-RNA: 2′-O-methyl RNA (2′-OMe): 2′-alkoxy-RNA: 2′-O-methoxyethyl-RNA (2′-MOE); 2′-amino-DNA; 2′-fluoro-RNA; 2′-fluoro-DNA; arabino nucleic acid (ANA): 2′-fluoro-ANA; or bicyclic nucleoside analog (LNA).

14. The ASO of claim 12, wherein the sugar modified nucleoside is an affinity enhancing 2′ sugar modified nucleoside.

15. (canceled)

16. The ASO of claim 14, wherein the affinity enhancing 2′ sugar modified nucleoside is an LNA, wherein the LNA is selected from the group consisting of constrained ethyl nucleoside (cEt), 2′,4′-constrained 2′-O-methoxyethyl (cMOE), α-L-LNA, β-D-LNA, 2′-O,4′-C-ethylene-bridged nucleic acids (ENA), amino-LNA, oxy-LNA, thio-LNA, or any combination thereof.

17. (canceled)

18. The ASO of claim 1, wherein the ASO comprises one or more 5′-methyl-cytosine nucleobases.

19. (canceled)

20. The ASO of claim 1, wherein the contiguous nucleotide sequence is complementary to a nucleic acid sequence comprising (i) nucleotides 625-842 of SEQ ID NO: 1; (ii) nucleotides 1,398-59,755 of SEQ ID NO: 1; (iii) nucleotides 61,817-104,725 of SEQ ID NO: 1; (iv) nucleotides 112,162-118,021 of SEQ ID NO: 1; (v) nucleotides 119,440-135,219 of SEQ ID NO: 1; (vi) nucleotides 137,587-157,856 of SEQ ID NO: 1; (vii) nucleotides 159,191-266,174 of SEQ ID NO: 1; or (viii) nucleotides 272,788-310,949 of SEQ ID NO: 1.

21-22. (canceled)

23. The ASO of claim 1, wherein the contiguous nucleotide sequence comprises SEQ ID NO: 4 to SEQ ID NO: 1713 with one or two mismatches.

24-32. (canceled)

33. The ASO of claim 1, wherein the contiguous nucleotide sequence comprises one or more modified internucleoside linkages.

34. The ASO of claim 33, wherein the one or more modified internucleoside linkages is a phosphorothioate linkage.

35-36. (canceled)

37. A conjugate comprising the ASO of claim 1, wherein the ASO is covalently attached to at least one non-nucleotide or non-polynucleotide moiety.

38. (canceled)

39. A pharmaceutical composition comprising the ASO of claim 1, and a pharmaceutically acceptable diluent, carrier, salt, or adjuvant.

40-43. (canceled)

44. A kit comprising the ASO of claim 1 and instructions for use.

45. (canceled)

46. A method of inhibiting or reducing CAMK2D protein expression in a cell, comprising administering the ASO of claim 1 to the cell expressing CAMK2D protein, wherein the CAMK2D protein expression in the cell is inhibited or reduced after the administration.

47. (canceled)

48. The method of claim 46, wherein the ASO inhibits or reduces expression of CAMK2D transcript (e.g., mRNA) in the cell after the administration.

49-51. (canceled)

52. A method of reducing, ameliorating, or treating one or more symptoms of a cardiovascular disease or disorder in a subject in need thereof, comprising administering an effective amount of the ASO of claim 1, to the subject.

53-56. (canceled)

57. The method of claim 52, wherein the cardiovascular disease or disorder comprises a coronary artery disease, stroke, heart failure, hypertensive heart disease, rheumatic heart disease, cardiomyopathy, heart arrhythmia, congenital heart disease, valvular heart disease carditis, aortic aneurysms, peripheral artery disease, thromboembolic disease, venous thrombosis, or any combination thereof.

58-61. (canceled)
